(19) United States
(12) Patent Application Publication
(10) Pub. No.: US 2023/0149356 A1
(43) Pub. Date: May 18, 2023

(54) NOVEL MEDICAL USE OF MDM2 INHIBITORS

(71) Applicants: ASCENTAGE PHARMA (SUZHOU) CO., LTD., Suzhou, Jiangsu (CN); ASCENTAGE PHARMA GROUP CORP LIMITED, Hong Kong (CN)

(72) Inventors: Yifan ZHAI, Suzhou, Jiangsu (CN); Dajun YANG, Suzhou, Jiangsu (CN)

(21) Appl. No.: 17/918,557

(22) PCT Filed: Apr. 13, 2021
(86) PCT No.: PCT/CN2021/086923

§ 371 Date: Oct. 12, 2022

Related U.S. Application Data
(60) Provisional application No. 63/009,465, filed on Apr. 14, 2020.

(51) Int. Cl. A61K 31/407; A61P 35/02; A61K 31/635; A61K 39/395; A61P 35/04; A61P 17/00

(52) U.S. Cl. CPC A61K 31/407; A61P 35/02; A61K 31/635; A61K 39/39558; A61P 35/04; A61P 17/00

(57) ABSTRACT

Provided use of MDM2 inhibitors alone or in combination with additional therapeutic agent in the treatment of conditions and diseases wherein inhibition of MDM2 and MDM2-related proteins provides a benefit.

### FIELD OF THE INVENTION

The present invention relates to use of MDM2 inhibitors alone or in combination with additional therapeutic agent in the treatment of conditions and diseases wherein inhibition of MDM2 and MDM2-related proteins provides a benefit.

### BACKGROUND OF THE INVENTION

MDM2 inhibitors interfere the binding of MDM2 oncoprotein with the tumor suppressor p53 protein, which thereby serves as a pharmacological p53 activator. Emerging evidence suggests that p53 dysfunction also fuels inflammation and supports tumor immune evasion and, thus, p53 dysfunction serves as an immunological driver of tumorigenesis (Guo G, Cancer Research, 2017; 77(9):2292).

MDM2 and p53 are part of an auto-regulatory feed-back loop (Wu et al., Genes Dev. 7:1126 (1993)). MDM2 is transcriptionally activated by p53 and MDM2, in turn, inhibits p53 activity by at least three mechanisms (Wu et al., Genes Dev. 7:1126 (1993)). First, MDM2 protein directly binds to the p53 transactivation domain, and thereby inhibits p53-mediated transactivation. Second, MDM2 protein contains a nuclear export signal sequence, and upon binding to p53, induces the nuclear export of p53, preventing p53 from binding to the targeted DNAs. Third, MDM2 protein is an E₃ ubiquitin ligase and upon binding to p53 is able to promote p53 degradation.

MDM2 inhibitors have been described previously as an anti-cancer therapeutic agent (See, e.g, U.S. Pat. No. 9,745,314, the entire contents of which are incorporated herein by reference), and is being evaluated in humans as mono-therapy or in combination with additional therapeutic agent for treatment of diseases and conditions wherein inhibition of MDM2 and MDM2-related proteins activity provides a benefit.

### SUMMARY OF THE INVENTION

It has now been found by the inventors of the present application that the administration of a MDM2 inhibitor, for example as a single agent, or in combination with a Bcl-2 inhibitor or a Bcl-2/Bcl-xL inhibitor, provides clinical benefits in the treatment of T-cell prolymphocytic leukemia (T-PLL). In addition, the administration of a MDM2 inhibitor and a modulator of an immune checkpoint molecule (e.g., an activator of a co-stimulatory molecule or an inhibitor of an immune checkpoint molecule) synergistically treats cancer.

In one aspect, provided herein is a method of treating T-cell prolymphocytic leukemia (T-PLL) in a subject in need thereof, comprising administering an effective amount of a MDM2 inhibitor.

In some embodiments, the MDM2 inhibitor is a compound of the following formula (VI), or a pharmaceutically acceptable salt thereof:

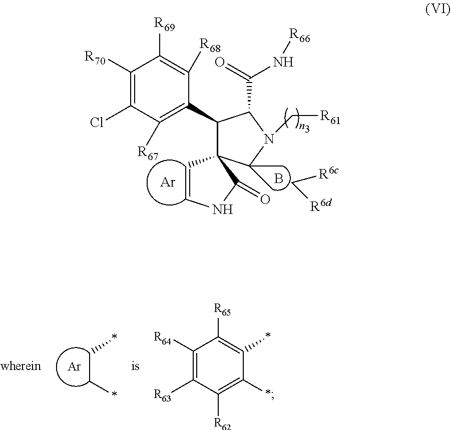

B is

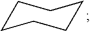

R₆₁ is H, or unsubstituted C₁₋₄ alkyl;
n₃ is 0, 1, or 2;
R₆₂, R₆₃, R₆₄, R₆₅, R₆₇, R₆₈, R₆₉, and R₇₀, independently, are selected from the group consisting of H, F, Cl, CH₃, and CF₃;
R₆₆ is and

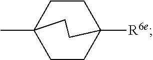

R^{6c} and R^{6d} are substituents on one carbon atom of ring B, wherein
R^{6c} is H, C₁₋₃ alkyl, C₁₋₃ alkylene-OR^{6a}, OR^{6a}, or halo;
R^{6d} is H, C₁₋₃ alkyl, C₁₋₃ alkylene-OR^{6a}, OR^{6a}, or halo;
R^{6e} is —C(═O)OR^{6a}, —C(═O)NR^{6a}R^{6b}, or —(═O)NHSO₂CH₃;
R^{6a} is hydrogen or unsubstituted C₁₋₄ alkyl; and
R^{6b} is hydrogen or unsubstituted C₁₋₄ alkyl.

In some embodiments, n₃ is 0, or 1.

In some embodiments, R₆₁ is H or CH₃.

In some embodiments,

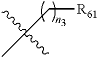

is H, CH₃, or CH₂CH₃.

In some embodiments, R₆₂ is H, R₆₃ is F or Cl, and R₆₄ and R₆₅ are H.

In some embodiments, R₆₇ is fluoro, each of R₆₈, R₆₉, and R₇₀ is H, R^{6c} is H, CH₃, OH, or halo, and R^{6d} is H, CH₃, OH, or halo.

In some embodiments, the MDM2 inhibitor is selected from the following group of compounds or a pharmaceutically acceptable salt thereof:

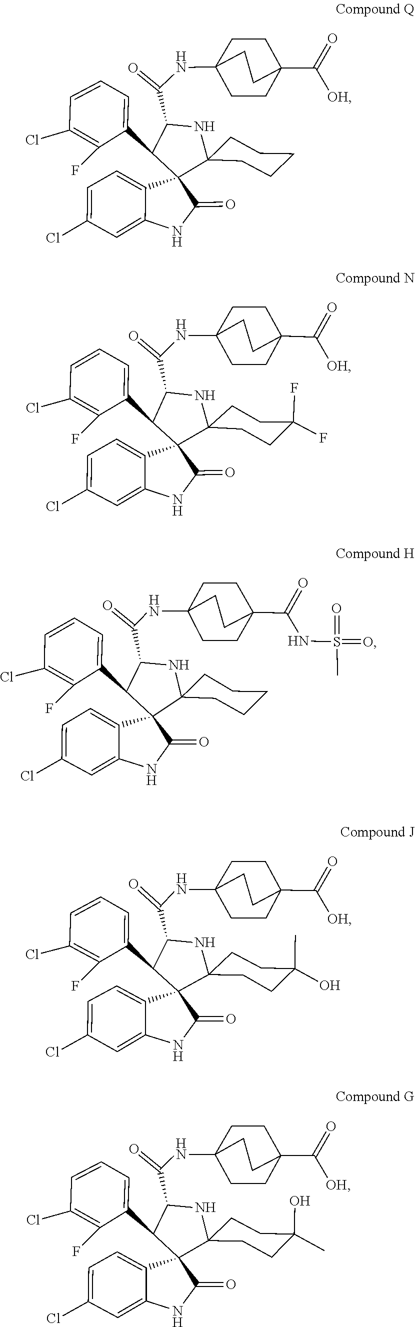 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

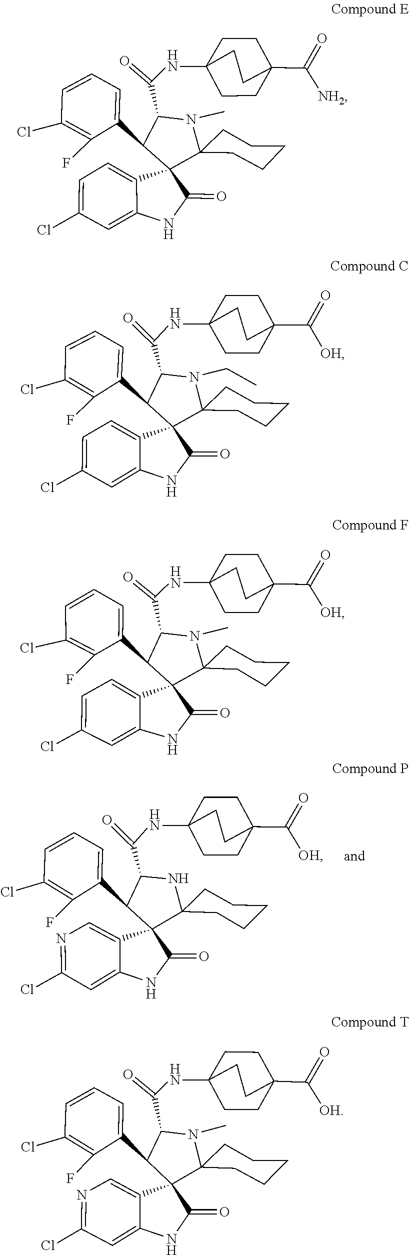 [SMILES: CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2]

In some embodiments, the MDM2 inhibitor is the compound having the following formula or a pharmaceutically acceptable salt thereof:

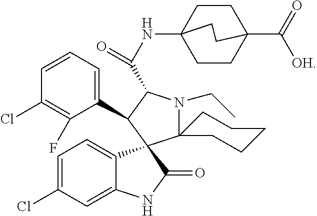 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

In some embodiments, the method further comprising administering an effective amount of a Bcl-2 inhibitor or a Bcl-2/Bcl-xL inhibitor.

In some embodiments, the Bcl-2 inhibitor or Bcl-2/Bcl-xL inhibitor is a compound of the following formula (I), (II), (IV) or (V), or a pharmaceutically acceptable salt thereof:

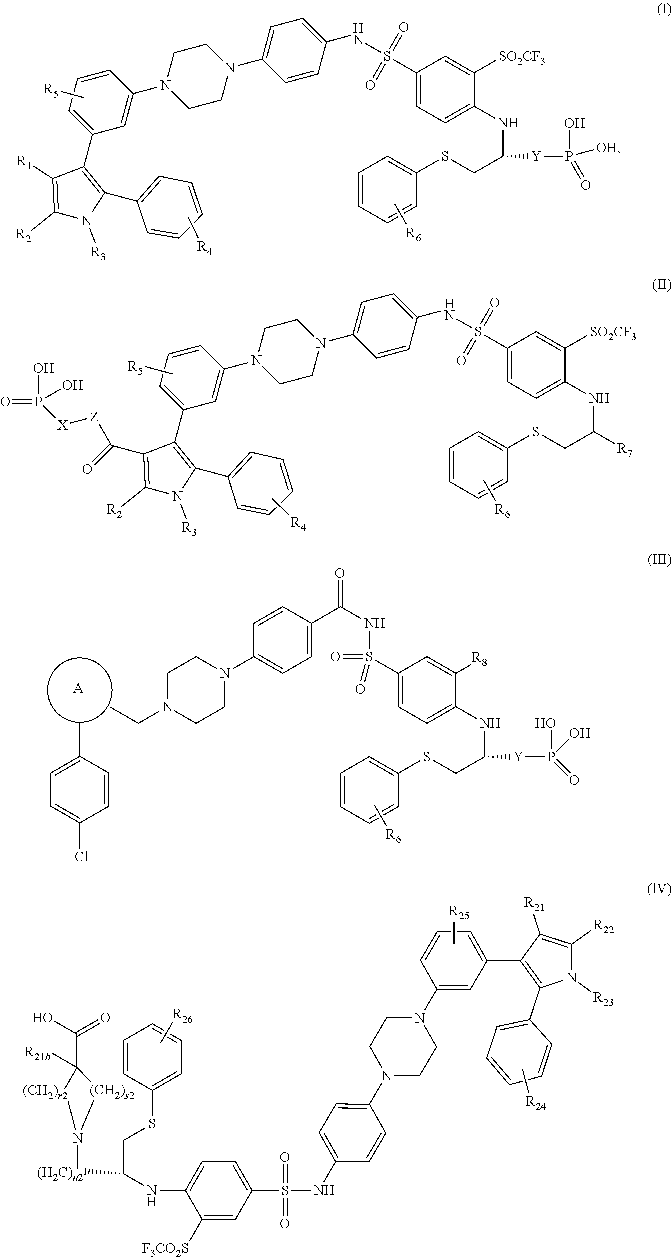

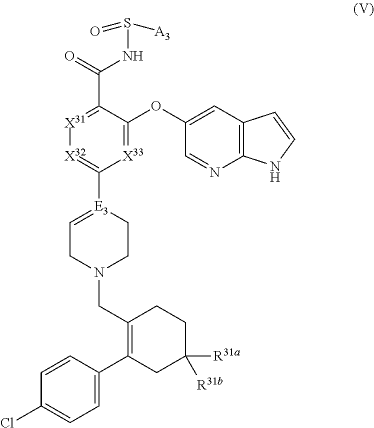

wherein the A ring is

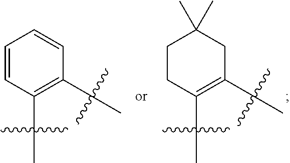

X, substituted or unsubstituted, is selected from the group consisting of alkylene, alkenylene, cycloalkylene, cycloalkenylene, and heterocycloalkylene;
Y is selected from the group consisting of (CH₂)ₙ—N(R^{a})₂ and

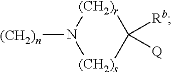

Q is selected from the group consisting of O, O(CH₂)₁₋₃, NR^{c}, NR^{c}(C₁₋₃ alkylene), OC(═O)(C₁₋₃ alkylene), C(═O)O, C(═O)O(C₁₋₃ alkylene), NHC(═O)(C₁₋₃ alkylene), C(═O)NH, and C(═O)NH(C₁₋₃ alkylene);
Z is O or NR^{c};
R₁ and R₂, independently, are selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, COR′, CO₂R′, OCOR′, CONR′R″, CONR′SO₂R″, NR′COR″, NR′CONR″R′″, NR′C═SNR″R″ NR'SO₂R″, SO₂R′, and SO₂NR′R″;
R₃ is selected from a group consisting of H, alkyl, cycloalkyl, alkenyl, cycloalkenyl, alkynyl, aryl, heteroaryl, heterocycloalkyl, OR′, NR′R″, OCOR′, CO₂R′, CUR, CONR′R″, CONR′SO₂R″, C₁₋₃ alkyleneCH(OH)CH₂OH, SO₂R′, and SO₂NR′R″;
R′, R″, and R′″, independently, are H, alkyl, cycloalkyl, alkenyl, cycloalkenyl, alkynyl, aryl, heteroaryl, C₁₋₃ alkyleneheterocycloalkyl, or heterocycloalkyl;
R′ and R″, or R″ and R′″, can be taken together with the atom to which they are bound to form a 3 to 7 membered ring;
R₄ is hydrogen, halo, C₁₋₃ alkyl, CF₃, or CN;
R₅ is hydrogen, halo, C₁₋₃alkyl, substituted C₁₋₃ alkyl, hydroxyalkyl, alkoxy, or substituted alkoxy;
R₆ is selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;
R₇, substituted or unsubstituted, is selected form the group consisting of hydrogen, alkyl, alkenyl, (CH₂)₀₋₃ cycloalkyl, (CH₂)₀₋₃ cycloalkenyl, (CH₂)₀₋₃ heterocycloalkyl, (CH₂)₀₋₃ aryl, and (CH₂)₀₋₃ heteroaryl;
R₈ is selected form the group consisting of hydrogen, halo, NO₂, CN, CF₃SO₂, and CF₃;
Rₐ is selected from the group consisting of hydrogen, alkyl, heteroalkyl, alkenyl, hydroxyalkyl, alkoxy, substituted alkoxy, cycloalkyl, cycloalkenyl, and heterocycloalkyl;
R_{b} is hydrogen or alkyl;
R_{c} is selected from the group consisting of hydrogen, alkyl, substituted alkyl, hydroxyalkyl, alkoxy, and substituted alkoxy;
n, r, and s, independently, are 1, 2, 3, 4, 5, or 6;
R₂₁ is SO₂R₂′,
R₂₂ is alkyl, preferably C1-C4 alkyl, more preferably methyl, propyl, or isopropyl,
R₂₃ is alkyl, preferably C1-C4 alkyl, more preferably methyl, propyl, or isopropyl,
R₂₄ is halogen, preferably fluoride, chloride,
R₂₅ is halogen, preferably fluoride, chloride,
R₂₆ is selected from H, halogen, alkyl, preferably fluoride, chloride, C1-C4 alkyl, more preferably methyl, propyl, isopropyl
R_{21b} is H or alkyl, preferably C1-C4 alkyl, more preferably methyl, propyl, or isopropyl,
n₂, r₂ and s₂ are independently 1, 2, 3, 4, 5 or 6, more preferably, r₂ and s₂ are both 2 and n₂ is 3, 4 or 5, more preferably, all of n₂, r₂ and s₂ are 2, and
R₂′ is alkyl, preferably C1-C4 alkyl, more preferably methyl, propyl, or isopropyl.
A₃ is selected from the group consisting of:

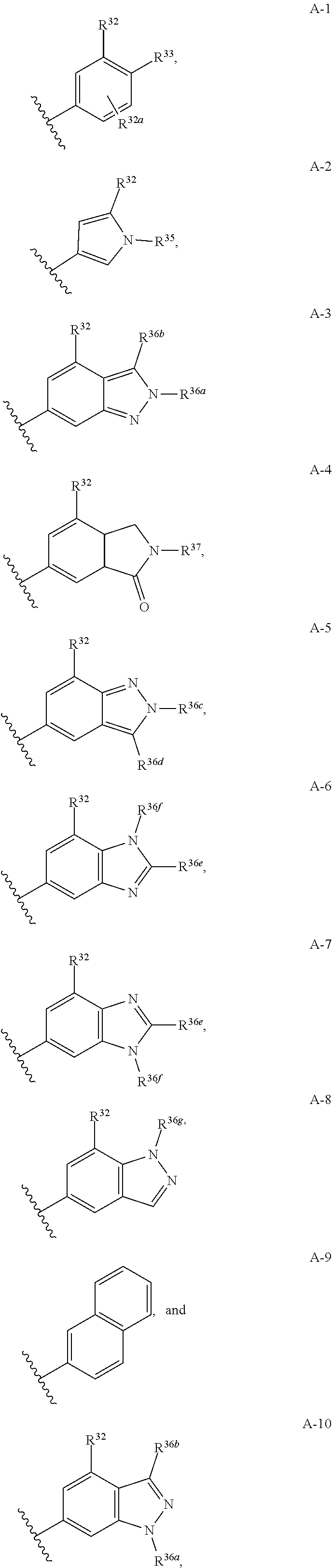

E₃ is a carbon atom and [symbol] is a double bond; or
E₃ is a —C(H)— and [symbol] is a single bond; or
E₃ is a nitrogen atom and [symbol] is a single bond;
X³¹, X³², and X³³ are each independently selected from the group consisting of —CR³⁸═ and —N═;
R^{31a} and R^{31b} taken together with the carbon atom to which they are attached form a 3-, 4-, or 5-membered optionally substituted cycloalkyl; or
R^{31a} and R^{31b} taken together with the carbon atom to which they are attached form a 4- or 5-membered optionally substituted heterocyclo;
R³² is selected from the group consisting of —NO₂, —SO₂CH₃, and —SO₂CF₃;
R^{32a} is selected from the group consisting of hydrogen and halogen;
R³³ is selected from the group consisting of hydrogen, —CN, and —N(R^{34a})(R^{34b});
R^{34a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{34b} is selected from the group consisting of hydrogen and C₁₋₄ alkyl;
R³⁵ is selected from the group consisting of is selected from the group consisting of optionally substituted C₁₋₆ alkyl, heterocyclo, heteroalkyl, (cycloalkyl)alkyl, and (heterocyclo)alkyl;
R^{36a}, R^{36c}, R^{36e}, R^{36f}, and R^{36g} are each independently selected from the group consisting of hydrogen, optionally substituted C₁₋₆ alkyl, optionally substituted C₃₋₆ cycloalkyl, optionally substituted aryl, optionally substituted heteroaryl, heterocyclo, heteroalkyl, (cycloalkyl)alkyl, and (heterocyclo)alkyl;
R^{36b} and R^{36d} are each independently selected from the group consisting of hydrogen, C₁₋₄ alkyl, and halogen;
R³⁷ is selected from the group consisting of optionally substituted C₁₋₆ alkyl, heterocyclo, heteroalkyl, (cycloalkyl)alkyl, and (heterocyclo)alkyl; and
R³⁸ is selected from the group consisting of hydrogen and halogen.

In some embodiments, the Bcl-2 inhibitor or the Bcl-2/Bcl-xL inhibitor is a compound selected from Table 1A, 1B, 1C, or a pharmaceutically acceptable salt thereof.

In some embodiments, the Bcl-2 inhibitor is a compound of the following formula, or a pharmaceutically acceptable salt thereof:

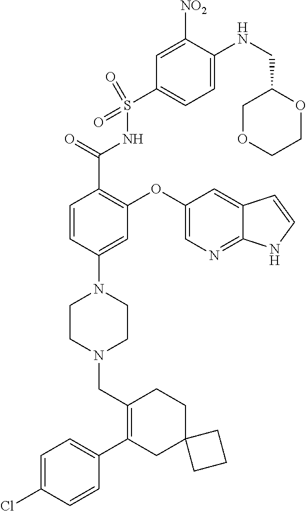 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In some embodiments, the MDM2 inhibitor is the compound having the following formula or a pharmaceutically acceptable salt thereof:

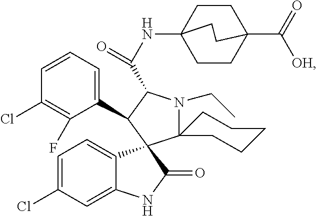 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

and
the Bcl-2 inhibitor is a compound of the following formula, or a pharmaceutically acceptable salt thereof

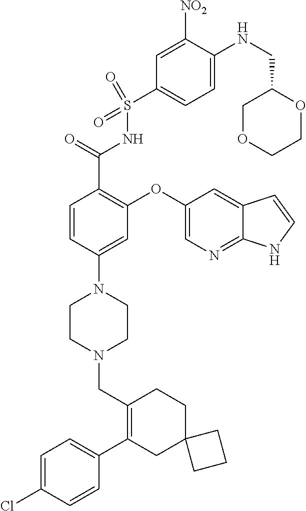 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In some embodiments, the MDM2 inhibitor is administered orally every day.

In some embodiments, the MDM2 inhibitor is administered orally in an effective amount from about 1 mg to about 300 mg every day, preferably in an amount of about 50 mg, 100 mg, 150 mg, 200 mg or 250 mg every day.

In some embodiments, the treatment comprises at least one 28-day treatment cycle, wherein the MDM2 inhibitor is administered orally every day in a patient in need thereof on days 1 to 5.

In some embodiments, the Bcl-2 inhibitor is administered at an effective amount from about 400 mg to about 1000 mg every day, preferably at about 400 mg, about 600 mg, or about 800 mg every day.

In some embodiments, the Bcl-2 inhibitor is administered orally once every day in the 28-day treatment cycle. In some embodiments, the method further comprises administering the Bcl-2 inhibitor according to a daily step-wise dosing regimen before initiation of the 28-day treatment cycle.

In some embodiments, the daily step-wise dosing regimen comprises administering the Bcl-2 inhibitor in a first dose of 20 mg to 100 mg for 1 day, and in a second dose of 50 mg to 200 mg for 1 day after the first dose. In some embodiments, the daily step-wise dosing regimen further comprises administering the Bcl-2 inhibitor in a third dose of 100 to 400 mg for 1 day after the second dose, and in a fourth dose of 200 mg to 800 mg for 1 to 7 days after the third dose.

In some embodiments, the daily step-wise dosing regimen comprises administering the Bcl-2 inhibitor in a first dose of 20 mg for 1 day, in a second dose of 50 mg for 1 day after the first dose, in a third dose of 100 mg for 1 day after the second dose, and in a fourth dose of 200 mg for 1 to 5 days after the third dose. In some embodiments, the fourth dose is administered for 1 day.

In some embodiments, the daily step-wise dosing regimen further comprises administering the Bcl-2 inhibitor in a fifth dose of 400 mg for 1 day after the fourth dose. In some embodiments, the daily step-wise dosing regimen further comprises administering the Bcl-2 inhibitor in a sixth dose of 600 mg for 1 day after the fifth dose.

In another aspect, provided herein is a method of treating a subject having cancer, comprising administering an effective amount of the MDM2 inhibitor as described herein and a modulator of an immune checkpoint molecule, wherein the subject has melanoma, non-small cell cancer (NSCLC), lung adenocarcinoma, solid tumor with wild-type p53 and ATM mutation, urothalial carcinoma and malignant peripheral nerve sheath tumor (MPNST).

In some embodiments, the MDM2 inhibitor is a compound selected from the following compound or a pharmaceutically acceptable salt thereof:

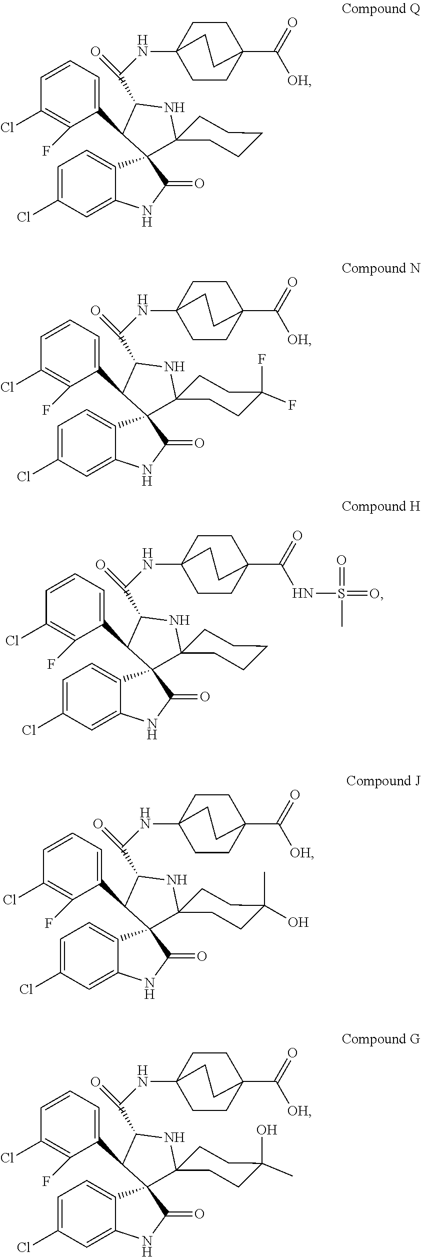 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

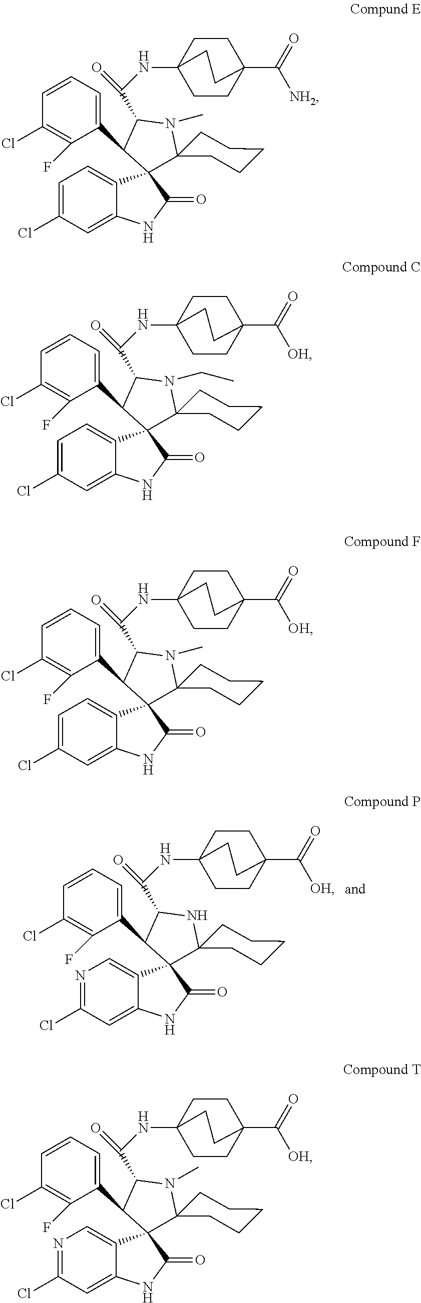 [SMILES: CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2]

In some embodiments, the MDM2 inhibitor is a compound having the following formula or a pharmaceutically acceptable salt thereof

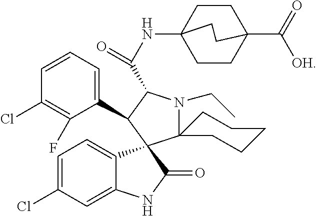 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

In some embodiments, the modulator of an immune checkpoint molecule is a modulator of the immune checkpoint molecule PD-1 or PD-L1.

In some embodiments, the modulator of the immune checkpoint molecule is a PD-1 or PD-L1 binding protein (e.g. anti-PD-1 antibody or anti-PD-L1 antibody).

In some embodiments, the modulator of the immune checkpoint molecule is selected from pembrolizumab, nivolumab, atezolizumab, avelumab, durvalumab, AMP-224, AMP-514, BGB-A₃₁₇, cemiplimab, JS001, CS1001, PDR-001, PF-06801591, pidilizumab, SHR-1210, and TSR-042.

In some embodiments, the modulator of the immune checkpoint molecule is pembrolizumab.

In some embodiments, the MDM2 inhibitor is a compound having the following formula or a pharmaceutically acceptable salt thereof

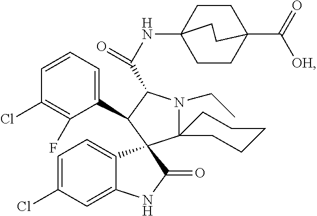 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

and the modulator of the immune checkpoint molecule is pembrolizumab.

In some embodiments, the MDM2 inhibitor is administered orally every other day.

In some embodiments, the MDM2 inhibitor is administered orally in an effective amount from about 1 mg to about 300 mg every day, preferably in an amount of about 50 mg, 100 mg, 150 mg, 200 mg, 250 mg every other day.

In some embodiments, the treatment comprises at least one 21-day treatment cycle, wherein the MDM2 inhibitor is administered orally every other day in a patient in need thereof for the first two consecutive weeks (e.g., on day 1, 3, 5, 7, 9, 11 and 13) of a 21-day treatment cycle and is not administered during the third week of the treatment cycle.

In some embodiments, pembrolizumab is administered via intravenous infusion in an amount of 200 mg on day 1 of the 21-day treatment cycle.

In some embodiments, the cancer is locally advanced, unresectable or metastatic.

In some embodiments, the subject is refractory or relapse of immunotherapy.

In some embodiments, the immunotherapy is anti-PD-1 or anti-PD-L1 therapy (such as treatment with anti-PD-1 or anti-PD-L1 antibody).

In some embodiments, the subject has unresectable or metastatic melanomas and is refractory or relapse after the treatment with anti-PD-1 or anti-PD-L1 antibody.

In some embodiments, the subject has unresectable or metastatic NSCLC and is refractory or relapse after the treatment with anti-PD-1 or anti-PD-L1 antibody.

In some embodiments, the subject has unresectable or metastatic lung adenocarcinoma with STK-11 mutation, and optionally is refractory or relapse after the treatment with anti-PD-1 or anti-PD-L1 antibody.

In some embodiments, the subject has unresectable or metastatic solid tumors with functional p53 (such as wild-type 53) and ATM mutation (such as germline ATM mutation or somatic ATM mutation).

In some embodiments, the subject has locally advanced or metastatic liposarcomas with functional p53 (such as wild-type p53) and MDM2 amplification.

In some embodiments, the subject has unresectable or metastatic urothelial carcinoma without FGFR translocation and/or point mutation and is refractory or relapse after the treatment with anti-PD-1 or anti-PD-L1 antibody.

In some embodiments, the subject has unresectable or metastatic MPNST.

### BRIEF DESCRIPTION OF THE DRAWINGS

FIG. 1 shows the treatment scheme of the Phase IIa study evaluating the pharmacokinetics, safety and efficacy of Compound C as a single agent in subjects with T-cell prolymphocytic leukemia (T-PLL).

### DETAILED DESCRIPTION OF THE INVENTION

The invention will now be described in connection with specific embodiments. It should be understood that the present disclosure is not limited to the following embodiments. Any changes or modifications may be made by those skilled in the art without departing from the scope of the invention, and such modifications or improvements are also included in the scope of the invention.

### I. Definitions

In accordance with the present disclosure and as used herein, the following terms are defined with the following meanings, unless explicitly stated otherwise. As used herein, the term “alkyl” refers to straight chain and branched saturated C₁₋₁₀ hydrocarbon groups, preferably C₁₋₆ hydrocarbon groups, non-limiting examples of which include methyl, ethyl, and straight chain and branched propyl, butyl, pentyl, hexyl, heptyl, octyl, nonyl, and decyl groups. The term Cₙ means the alkyl group has “n” carbon atoms. The term Cₙ₋ₚ means that the alkyl group contains “n” to “p” carbon atoms.

The term “alkylene” refers to an alkyl group having a substituent. An alkyl, e.g., methyl, or alkylene, e.g., —CH₂—, group can be substituted with one or more, and typically one to three, of independently selected halo, trifluoromethyl, trifluoromethoxy, hydroxy, alkoxy, nitro, cyano, alkylamino, or amino groups, for example.

The term “alkenyl” is defined identically as “alkyl,” except for containing a carbon-carbon double bond, e.g., ethenyl, propenyl, and butenyl. The term “alkenylene” is defined identically to “alkylene” except for containing a carbon-carbon double bond. The term “alkynyl” and “alkynylene” are defined identically as “alkyl” and “alkylene” except the group contains a carbon-carbon triple bond.

As used herein, the term “halo” is defined as fluoro, chloro, bromo, or iodo.

The term “hydroxy” is defined as —OH.

The term “alkoxy” is defined as —OR, wherein R is alkyl.

The term “amino” is defined as —NH₂, and the term “alkylamino” is defined as —NR², wherein at least one R is alkyl and the second R is alkyl or hydrogen.

The term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)—, alkyl(aralkyl)-NC(O)— and the like.

The term “carboxy” is defined as —C(═O)OH or a salt thereof.

The term “nitro” is defined as —NO₂.

The term “cyano” is defined as —CN.

The term “trifluoromethyl” is defined as —CF₃.

The term “trifluoromethoxy” is defined as —OCF₃.

As used herein, the term “aryl” refers to a monocyclic or polycyclic aromatic group, preferably a monocyclic or bicyclic aromatic group. Examples of aryl groups include, but are not limited to, phenyl, naphthyl, fluorenyl, azulenyl, anthryl, phenanthryl, pyrenyl, biphenyl, and terphenyl. Aryl also refers to bicyclic and tricyclic carbon rings, where one ring is aromatic and the others are saturated, partially unsaturated, or aromatic, for example, dihydronaphthyl, indenyl, indanyl, or tetrahydronaphthyl (tetralinyl). Unless otherwise indicated, an aryl group can be unsubstituted or substituted with one or more, and in particular one to four, groups independently selected from, for example, halo, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “heterocyclic” refers to a heteroaryl and heterocycloalkyl ring systems.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “cycloalkyl” means a monocyclic or bicyclic, saturated or partially unsaturated, ring system containing three to eight carbon atoms, including cyclopropyl, cyclobutyl, cyclopentyl, cyclohexyl, cycloheptyl, and cyclooctyl, optionally substituted with one or more, and typically one to three, of independently selected halo, trifluoromethyl, trifluoromethoxy, hydroxy, alkoxy, nitro, cyano, alkylamino, or amino groups, for example.

As used herein, the term “heterocycloalkyl” means a monocyclic or bicyclic ring system containing at least one nitrogen, oxygen, or sulfur atom in the ring system. Non-limiting examples of heterocycloalkyl groups are azetidinyl, pyrrolidinyl, piperidinyl, piperazinyl, dihydropyrrolyl, morpholinyl, thiomorpholinyl, dihydropyridinyl, oxacycloheptyl, dioxacycloheptyl, thiacycloheptyl, diazacycloheptyl, each optionally substituted with one or more, and typically one to three, of independently selected halo, C₁₋₆ alkyl, C₁₋₆ alkoxy, cyano, amino, carbamoyl, nitro, carboxy, C₂₋₇ alkenyl, C₂₋₇ alkynyl, or the like on an atom of the ring.

As used herein, the term “pharmaceutically acceptable salts” refers to salts or zwitterionic forms of the compounds of the present disclosure. Salts of compounds of the disclosure can be prepared during the final isolation and purification of the compounds or separately by reacting the compound with an acid having a suitable cation. The pharmaceutically acceptable salts of compounds of the present disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the invention include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides.

Additionally, salts, hydrates, solvates and active metabolites of the compounds of the present disclosure are included in the present disclosure and can be used in the methods disclosed herein. The present disclosure further includes all possible stereoisomers and geometric isomers of the compounds of the present disclosure. The present disclosure includes both racemic compounds and optically active isomers. When a compound of the present disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the compounds of the present disclosure are possible, the present disclosure is intended to include all tautomeric forms of the compounds.

In accordance with the foregoing, any reference to a compound of the invention presented herein is intended to include a compound of formula (I), (II), (III), (IV), (V) or (VI), and pharmaceutically acceptable salts, hydrates, solvates or active metabolite thereof. The term “active metabolite” as used herein refers to a metabolite of a compound of the present disclosure in the human body.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the terms “treat,” “treating” or “treatment” refer, preferably, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of progression or increasing time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, treatment does not include one or more of a decrease in pain or an increase in the quality of life (QOL) as judged by a qualified individual, e.g., a treating physician, e.g., using accepted assessment tools of pain and QOL.

In certain embodiments, treatment includes a symptom or sign which approaches a normalized value (for example a value obtained in a healthy patient or individual), e.g., is less than 50% different from a normalized value, in certain embodiments less than about 25% different from a normalized value, in other embodiments is less than 10% different from a normalized value, and in yet other embodiments the presence of a symptom is not significantly different from a normalized value as determined using routine statistical tests. As used herein, treatment can include reduction of tumor burden, inhibition of tumor growth, including inducing stable disease in a subject with progressive disease prior to treatment, increasing time to progression, or increasing survival time. Increases can be determined relative to an appropriate control or expected outcomes. As used herein, treatment can include increasing survival of a subject, with or without a decrease in tumor burden, as compared to appropriate controls. Treatment need not be curative.

As used herein, “co-administration” or “combination” with regard to a therapy or treatment is understood as administration of two or more active agents using separate formulations or a single pharmaceutical formulation such that there is a time period while both (or all) active agents simultaneously exert their biological activities. It is contemplated herein that one active agent can improve the activity of a second agent, for example, can sensitize target cells, e.g., cancer cells, to the activities of the second agent. Co-administration does not require that the agents are administered at the same time, at the same frequency, or by the same route of administration. As used herein, “co-administration” or “combination therapy” includes administration of a MDM2 inhibitor with one or more additional therapeutic agent.

The terms “administer”, “administering” or “administration” include any method of delivery of a pharmaceutical composition or agent into a subject's system or to a particular region in or on a subject. In certain embodiments, the agent is delivered orally. In certain embodiments, the agent is administered parenterally. In certain embodiments, the agent is delivered by injection or infusion. In certain embodiments, the agent is delivered topically including transmucosally. In certain embodiments, the agent is delivered by inhalation. In certain embodiments of the invention, an agent is administered by parenteral delivery, including, intravenous, intramuscular, subcutaneous, intramedullary injections, as well as intrathecal, direct intraventricular, intraperitoneal, intranasal, or intraocular injections. In one embodiment, the compositions provided herein may be administered by injecting directly to a tumor. In some embodiments, the formulations of the invention may be administered by intravenous injection or intravenous infusion. In certain embodiments, the formulation of the invention can be administered by continuous infusion. In certain embodiments, administration is not oral. In certain embodiments, administration is systemic. In certain embodiments, administration is local. In some embodiments, one or more routes of administration may be combined, such as, for example, intravenous and intratumoral, or intravenous and peroral, or intravenous and oral, intravenous and topical, or intravenous and transdermal or transmucosal. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, continuous infusion, etc.

A “subject” to be treated by the method of the invention can mean either a human or non-human animal, preferably a mammal, more preferably a human. In certain embodiments, a subject has a detectable tumor prior to initiation of treatments using the methods of the invention. In certain embodiments, the subject has a detectable tumor at the time of initiation of the treatments using the methods of the invention.

The term “therapeutically effective amount” or “effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” or “effective amount” will vary depending on the compound, the disease and its severity and the age, weight, etc., of the patient to be treated. A therapeutically effective amount or an effective amount need not be curative. A therapeutically effective amount or an effective amount need not prevent a disease or condition from ever occurring. Instead a therapeutically effective amount or an effective amount is an amount that will at least delay or reduce the onset, severity, or progression of a disease or condition. Disease progression can be monitored, for example, by one or more of tumor burden, time to progression, survival time, or other clinical measurements used in the art.

As used herein, “cancer” or “tumor” is a generic name for a wide range of cellular malignancies characterized by unregulated growth, lack of differentiation, and the potential or ability to invade local tissues and metastasize. These neoplastic malignancies affect, with various degrees of prevalence, every tissue and organ in the body. Cancer involves presence of cells possessing characteristics typical of cancer-causing cells, such as uncontrolled proliferation, immortality, metastatic potential, rapid growth and proliferation rate, and certain characteristic morphological features. Often, cancer cells will be in the form of a mass of tissue, but such cells may exist alone or may circulate in the blood stream as independent cells, such as leukemic cells.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Ranges expressed as “up to” a certain value, e.g., up to 5, is understood as all values, including the upper limit of the range, e.g., 0, 1, 2, 3, 4, and 5, and fractional values as appropriate. Up to or within a week is understood to include, 0.5, 1, 2, 3, 4, 5, 6, or 7 days. Similarly, ranges delimited by “at least” are understood to include the lower value provided and all higher numbers.

All percent formulations are w/w unless otherwise indicated.

As used herein, “about” is understood to include within three standard deviations of the mean or within standard ranges of tolerance in the specific art. In certain embodiments, about is understood a variation of no more than 0.5.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to”.

The term “or” is used inclusively herein to mean, and is used interchangeably with, the term “and/or,” unless context clearly indicates otherwise.

The term “such as” is used herein to mean, and is used interchangeably, with the phrase “such as but not limited to”.

### II. Methods of Treating T-PLL

Provided herein are methods of treating T-cell prolymphocytic leukemia (T-PLL) in a subject in need thereof, comprising administering an effective amount of a MDM2 inhibitor of the present invention to the subject.

In some embodiments, the methods of treating T-PLL further comprise administering an effective amount of a Bcl-2 inhibitor or a Bcl-2/Bcl-xL inhibitor to the subject.

T-cell-prolymphocytic leukemia (T-PLL) is a rare, mature T-cell leukemia with aggressive behavior, typically with the involvement of peripheral blood, bone marrow, lymph nodes, liver, spleen, and skin. T-PLL patients usually have a poor prognosis, due to a poor response to conventional chemotherapy. Monoclonal antibody therapy with anti-CD52 alemtuzumab combined with stem cell transplantation (SCT) has been reported to considerably improve outcomes to extend the median survival to four years or more (Laribi K, et al., Oncotarget, (2017)8:104664). Nevertheless, it is desirable to develop effective therapeutic strategy for T-PLL to improve clinical responses.

### a) MDM2 Inhibitors

The MDM2 inhibitors disclosed in the present invention inhibit the interaction between p53 or p53-related proteins and MDM2 or MDM2-related proteins. By inhibiting the negative effect of MDM2 or MDM2-related proteins on p53 or p53-related proteins, the MDM2 inhibitors of the present invention sensitize cells to inducers of apoptosis and/or cell cycle arrest. In one embodiment, the MDM2 inhibitors of the present invention induce apoptosis and/or cell cycle arrest.

“MDM2” as used herein is short for Murine Double Minute 2. The term MDM2 is can encompass the MDM2 gene, as well as the MDM2 gene product (e.g. mRNA, protein). Exemplary sequence of human MDM2 is available under the NCBI accession number of ABT17086, ABT17084.1, ABT17085.1, or ABT17083.1.

The term “MDM2-related protein,” as used herein, refers to proteins that have at least 25% sequence homology with MDM2, and interact with and inhibit p53 or p53-related proteins. Examples of MDM2-related proteins include, but are not limited to, MDMX.

The term “TP53” and “p53” are used interchangeably herein, and can refer to the p53 gene or gene product (e.g. mRNA, protein). Exemplary sequence of human p53 is available in UniProtKB database under the accession number of P04637 (P53-HUMAN) or under the NCBI accession number of AYF55702.1, or AXU92429.1.

The term “p53-related protein,” as used herein, refers to proteins that have at least 25% sequence homology with p53, have tumor suppressor activity, and are inhibited by interaction with MDM2 or MDM2-related proteins. Examples of p53-related proteins include, but are not limited to, p63 and p73.

In certain embodiments, the MDM2 inhibitor is compound of the following formula (VI) or a pharmaceutically acceptable salt thereof:

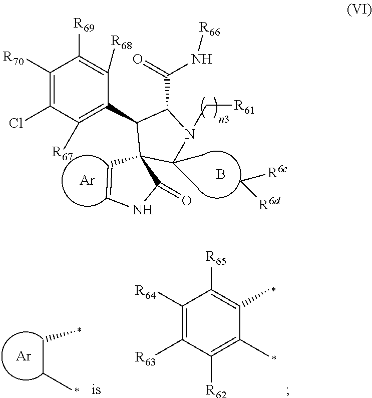

B is

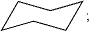

R₆₁ is H, or unsubstituted C₁₋₄ alkyl;
n₃ is 0, 1, or 2,
R₆₂, R₆₃, R₆₄, R₆₅, R₆₇, R₆₈, R₆₉, and R₇₀, independently, are selected from the group consisting of H, F, Cl, CH₃, and CF₃;
R₆₆ is

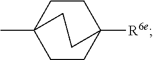

and
R^{6c} and R^{6d} are substituents on one carbon atom of ring B, wherein
R^{6c} is H, C₁₋₃ alkyl, C₁₋₃ alkylene-OR^{6a}, OR^{6a}, or halo;
R^{6d} is H, C₁₋₃ alkyl, C₁₋₃ alkylene-OR^{6a}, OR^{6a}, or halo;
R^{6e} is —C(═O)OR^{6a}, —C(═O)NR^{6a}R^{6b}, or —C(═O)NHSO₂CH₃;
R^{6a} is hydrogen or unsubstituted C₁₋₄ alkyl; and
R^{6b} is hydrogen or unsubstituted C₁₋₄ alkyl.

The compounds of structural formula (VI) inhibit MDM2-p53 interactions and are useful in the treatment of a variety of diseases and conditions. In particular, the compounds of structural formula (VI) are used in methods of treating a disease or condition wherein inhibition of MDM2 and MDM2-related protein provides a benefit, for example, cancers and proliferative diseases. The method comprises administering a therapeutically effective amount of a compound of structural formula (VI) to a subject in need thereof.

In some embodiments, n₃ is 0, or 1.

In some embodiments, R₆₁ is H or CH₃.

In some embodiments,

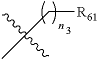

is H, CH₃, or CH₂CH₃.

In some embodiments, R₆₂ is H, R₆₃ is F or Cl, and R₆₄ and R₆₅ are H.

In some embodiments, R₆₇ is fluoro, each of R₆₈, R₆₉, and R₇₀ is H, R^{6c} is H, CH₃, OH, or halo, and R^{6d} is H, CH₃, OH, or halo.

In some embodiments, the MDM2 inhibitor is selected from the following compound or a pharmaceutically acceptable salt thereof:

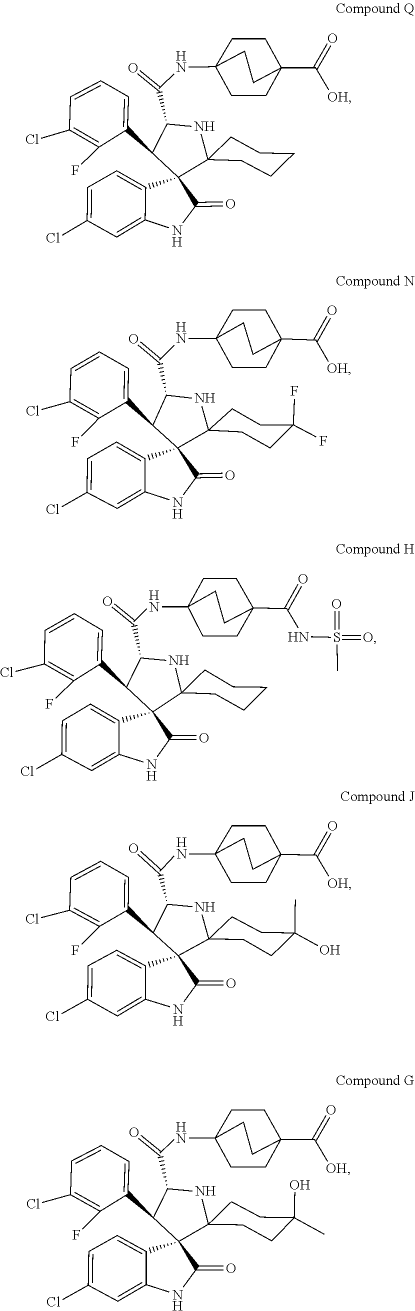 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

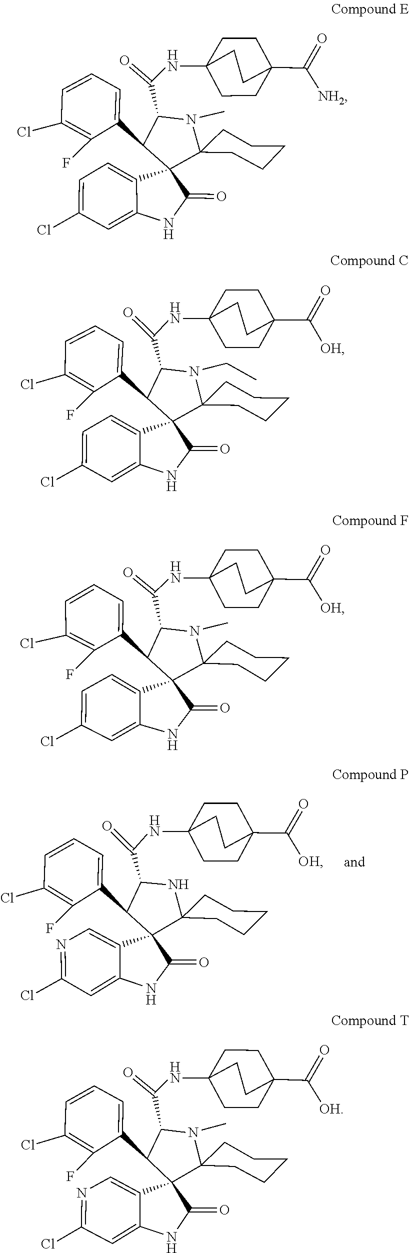 [SMILES: CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2]

In one embodiment, the MDM2 inhibitor is 4-((3′R,4′S,5′R)-6″-Chloro-4′-(3-chloro-2-fluorophenyl)-1′-ethyl-2″-oxodispiro[cyclohexane-1,2′-pyrrolidine-3′,3″-indoline]-5′-carboxamido)bicyclo[2.2.2]octane-1-carboxylic acid (“Compound C”) having the following formula

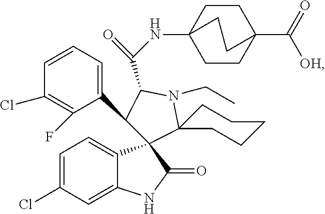 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically acceptable salt thereof.

More MDM2 inhibitors and the synthesis of the MDM2 inhibitors that can be used in the present application are further disclosed in U.S. Pat. No. 9,745,314, which is incorporated herein by reference.

### b) Bcl-2 Inhibitors or Bcl-2/Bcl-xL Inhibitors

In certain embodiments, the method of treating T-PLL further comprises administering an effective amount of a Bcl-2 inhibitor or a Bcl-2/Bcl-xL inhibitor.

As used herein, the term “Bcl-2/Bcl-xL” means Bcl-2, Bcl-xL, or Bcl-2 and Bcl-xL, i.e., Bcl-2 and/or Bcl-xL.

Bcl-2 family proteins are important regulators of apoptosis, which is a natural pathway for the body to clear abnormal or unwanted cells. The family of proteins includes anti-apoptotic proteins such as Bcl-2, Bcl-xL and Mcl-1; and pro-apoptotic molecules, including Bid, Bim, Bad, Bak and Bax. Although normal cells have low expression levels of anti-apoptotic Bcl-2 and Bcl-xL proteins, these proteins are found to be highly overexpressed in many different types of human tumors and are implied in tumor development, progression and resistance to drugs. Targeting Bcl-2 and/or Bcl-xL has been investigated as a cancer treatment strategy.

The Bcl-2/Bcl-xL inhibitor of the present disclosure is a compound of the following formula (I), (II) or (III), or a pharmaceutically acceptable salt thereof:

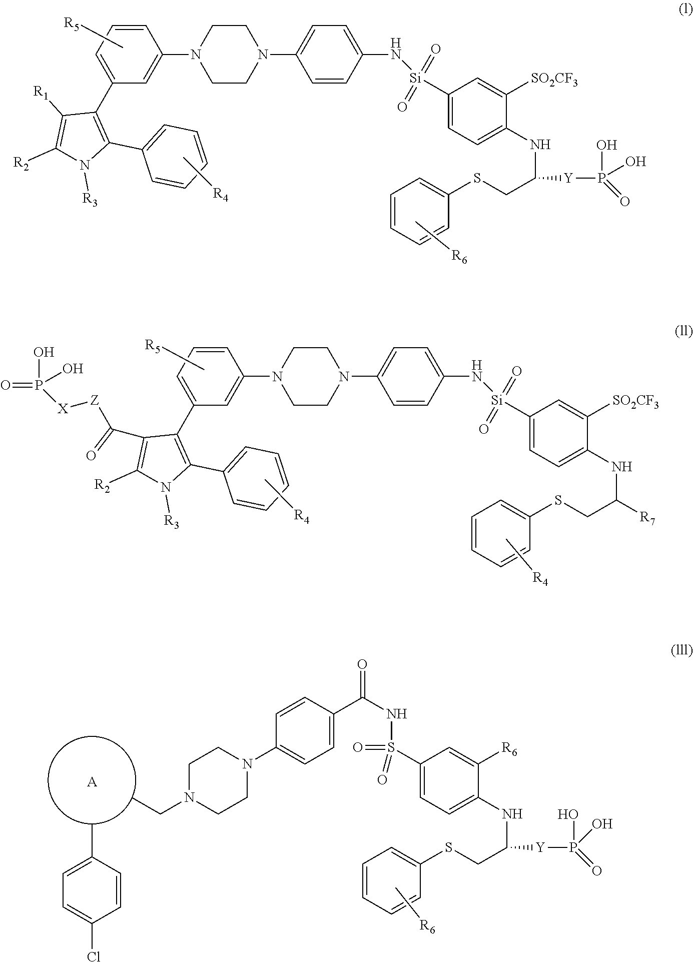

wherein the A ring is

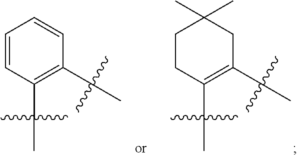

X, substituted or unsubstituted, is selected from the group consisting of alkylene, alkenylene, cycloalkylene, cycloalkenylene, and heterocycloalkylene;
Y is selected from the group consisting of (CH₂)ₙ—N(R^{a})₂ and

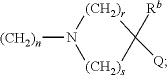

Q is selected from the group consisting of O, O(CH₂)₁₋₃, NR^{c}, NR^{c}(C₁₋₃ alkylene), OC(═O)(C₁₋₃ alkylene), C(═O)O, C(═O)O(C₁₋₃ alkylene), NHC(═O)(C₁₋₃ alkylene), C(═O)NH, and C(═O)NH(C₁₋₃ alkylene);

### Z is O or NR^{c};

R₁ and R₂, independently, are selected from the group consisting of H, CN, NO₂, halo, alkyl, cycloalkyl, alkenyl, cycloalkenyl, alkynyl, aryl, heteroaryl, heterocycloalkyl, SR, NRR″, COR′, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;
R₃ is selected from a group consisting of H, alkyl, cycloalkyl, alkenyl, cycloalkenyl, alkynyl, aryl, heteroaryl, heterocycloalkyl, OR′, NRR″, OCOR′, CO₂R′, COR′, CONR′R″, CONR′SO₂R″, C₁₋₃ alkyleneCH(OH)CH₂OH, SO₂R′, and SO₂NR′R″;
R′, R″, and R′″, independently, are H, alkyl, cycloalkyl, alkenyl, cycloalkenyl, alkynyl, aryl, heteroaryl, C₁₋₃alkyleneheterocycloalkyl, or heterocycloalkyl;
R′ and R″, or R″ and R′″, can be taken together with the atom to which they are bound to form a 3 to 7 membered ring;
R₄ is hydrogen, halo, C₁₋₃alkyl, CF₃, or CN;
R₅ is hydrogen, halo, C₁₋₃ alkyl, substituted C₁₋₃ alkyl, hydroxyalkyl, alkoxy, or substituted alkoxy;
R₆ is selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;
R₇, substituted or unsubstituted, is selected form the group consisting of hydrogen, alkyl, alkenyl, (CH₂)₀₋₃ cycloalkyl, (CH₂)₀₋₃ cycloalkenyl, (CH₂)₀₋₃heterocycloalkyl, (CH₂)₀₋₃ aryl, and (CH₂)₀₋₃ heteroaryl;
R₈ is selected form the group consisting of hydrogen, halo, NO₂, CN, CF₃SO₂, and CF₃;
Rₐ is selected from the group consisting of hydrogen, alkyl, heteroalkyl, alkenyl, hydroxyalkyl, alkoxy, substituted alkoxy, cycloalkyl, cycloalkenyl, and heterocycloalkyl;
R_{b} is hydrogen or alkyl;
R_{c} is selected from the group consisting of hydrogen, alkyl, substituted alkyl, hydroxyalkyl, alkoxy, and substituted alkoxy; and
n, r, and s, independently, are 1, 2, 3, 4, 5, or 6.

In some embodiments, the Bcl-2/Bcl-xL inhibitor described herein is a compound having the structure of formula (IV), or a pharmaceutically acceptable salt thereof,

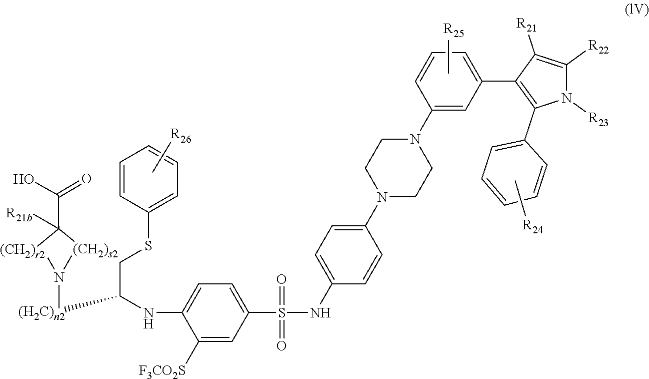

wherein R₂₁ is SO₂R₂′,
R₂₂ is alkyl, preferably C₁₋₄ alkyl, more preferably methyl, propyl, or isopropyl,
R₂₃ is alkyl, preferably C₁₋₄ alkyl, more preferably methyl, propyl, or isopropyl,
R₂₄ is halogen, preferably fluoride, chloride,
R₂₅ is halogen, preferably fluoride, chloride,
R₂₆ is selected from H, halogen, alkyl, preferably fluoride, chloride, C₁₋₄ alkyl, more preferably methyl, propyl, isopropyl,
R_{21b} is H or alkyl, preferably C₁₋₄ alkyl, more preferably methyl, propyl, or isopropyl,
n₂, r₂ and s₂ are independently 1, 2, 3, 4, 5 or 6, more preferably, r₂ and s₂ are both 2 and n₂ is 3, 4 or 5, more preferably, all of n₂, r₂ and s₂ are 2; and
R₂′ is alkyl, preferably C₁₋₄ alkyl, more preferably methyl, propyl, or isopropyl.

In some embodiments, the Bcl-2 inhibitor described herein is a compound having the structure of formula (V), or a pharmaceutically acceptable salt thereof,

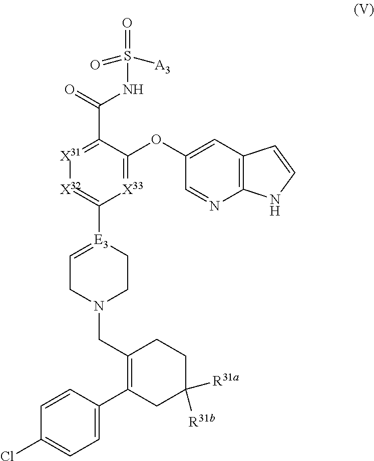

wherein A₃ is selected from the group consisting of:

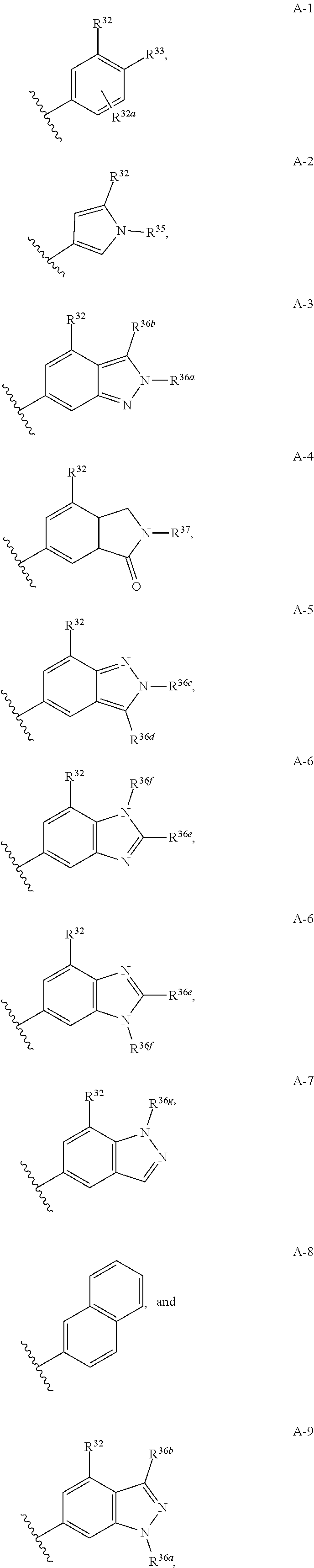

E₃ is a carbon atom and [symbol] is a double bond; or
E₃ is a —C(H)— and [symbol] is a single bond; or
E₃ is a nitrogen atom and [symbol] is a single bond;
X³¹, X³², and X³³ are each independently selected from the group consisting of —CR³⁸═ and —N═;
R^{31a} and R^{31b} taken together with the carbon atom to which they are attached form a 3-, 4-, or 5-membered optionally substituted cycloalkyl; or
R^{31a} and R^{31b} taken together with the carbon atom to which they are attached form a 4- or 5-membered optionally substituted heterocyclo;
R³² is selected from the group consisting of —NO₂, —SO₂CH₃, and —SO₂CF₃;
R^{32a} is selected from the group consisting of hydrogen and halogen;
R³³ is selected from the group consisting of hydrogen, —CN, —C≡CH, and —N(R₃₄ₐ)(R^{34b});
R₃₄ₐ is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{34b} is selected from the group consisting of hydrogen and C₁₋₄ alkyl;
R³⁵ is selected from the group consisting of is selected from the group consisting of optionally substituted C₁₋₆ alkyl, heterocyclo, heteroalkyl, (cycloalkyl)alkyl, and (heterocyclo)alkyl;
R^{36a}, R^{36c}, R^{36e}, R^{36f}, and R^{36g} are each independently selected from the group consisting of hydrogen, optionally substituted C₁₋₆ alkyl, optionally substituted C₃₋₆ cycloalkyl, optionally substituted aryl, optionally substituted heteroaryl, heterocyclo, heteroalkyl, (cycloalkyl)alkyl, and (heterocyclo)alkyl;
R^{36b} and R^{36d} are each independently selected from the group consisting of hydrogen, C₁₋₄ alkyl, and halogen;
R³⁷ is selected from the group consisting of optionally substituted C₁₋₆ alkyl, heterocyclo, heteroalkyl, (cycloalkyl)alkyl, and (heterocyclo)alkyl; and
R³⁸ is selected from the group consisting of hydrogen and halogen.

Specific compounds of the Bcl-2/Bcl-xL inhibitor or Bcl-2 inhibitor provided herein include, but are not limited to, compounds having the structure formulae set forth below in Table 1A to 1C, or a pharmaceutically acceptable salt thereof.

In certain embodiments, the Bcl-2/Bcl-xL inhibitors described herein having a structural formula (I), (II) or (III) are selected from Table 1A.

TABLE 1A
Exemplary compounds of Formula (I), (II) or (III)

| Compound | Structure |
|---|---|
| A1 | 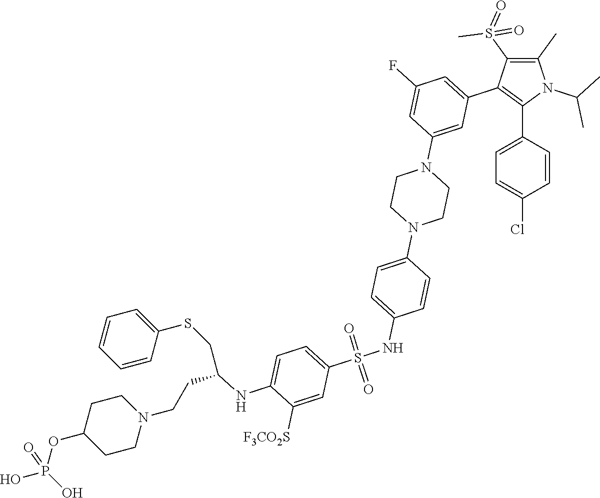 |
| A2 | 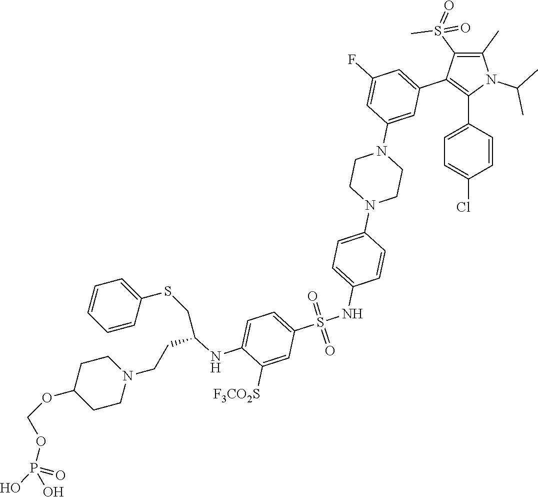 |
| A3 | 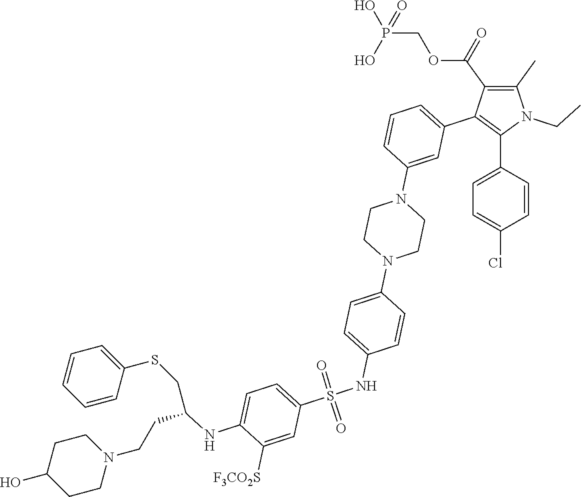 |
| A4 | 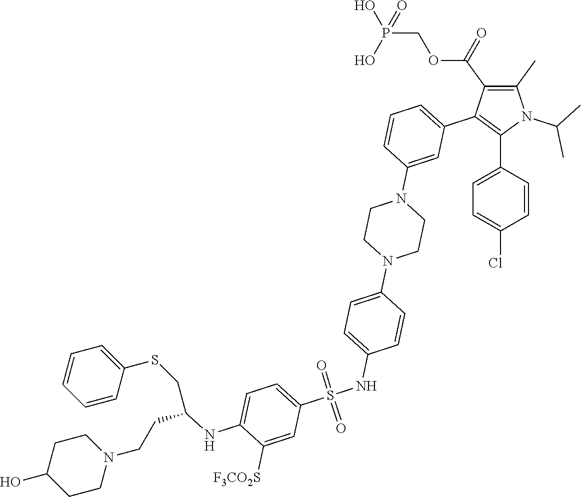 |
| A5 | 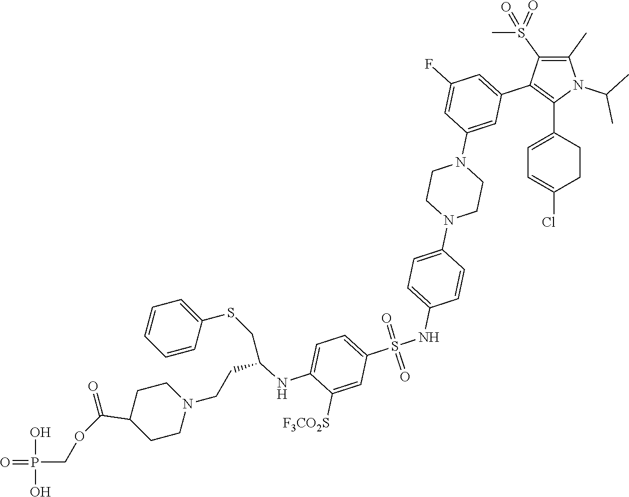 |
| A6 | 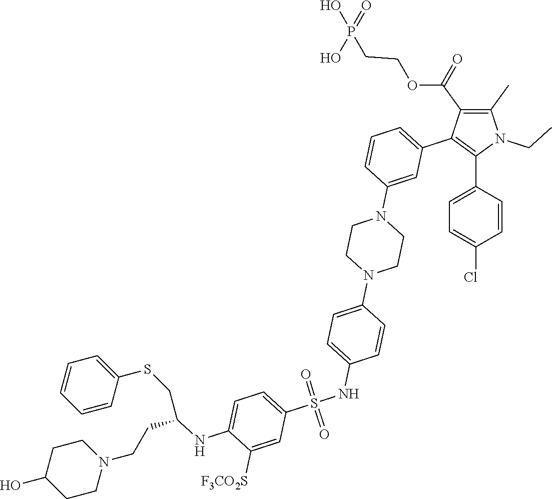 |
| A7 | 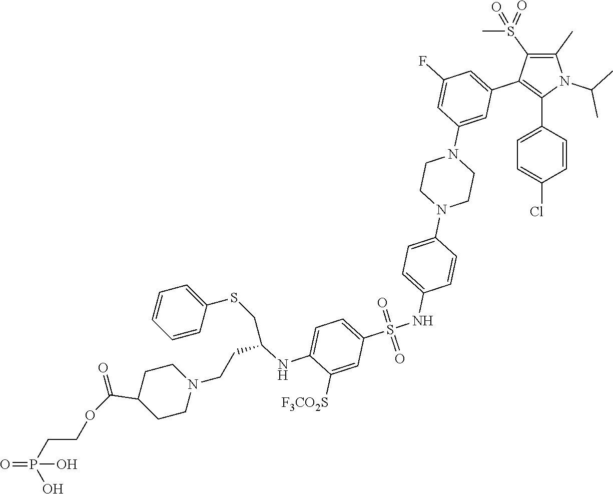 |
| A8 | 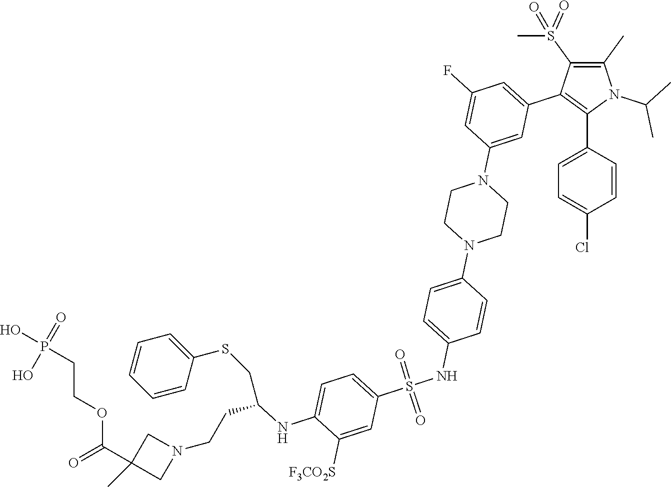 |
| A9 | 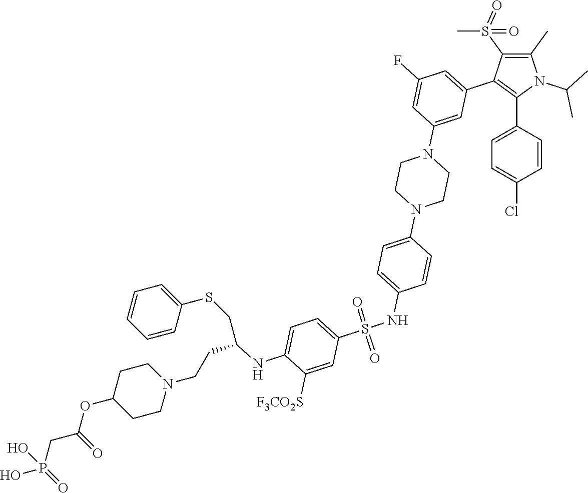 |
| A10 | 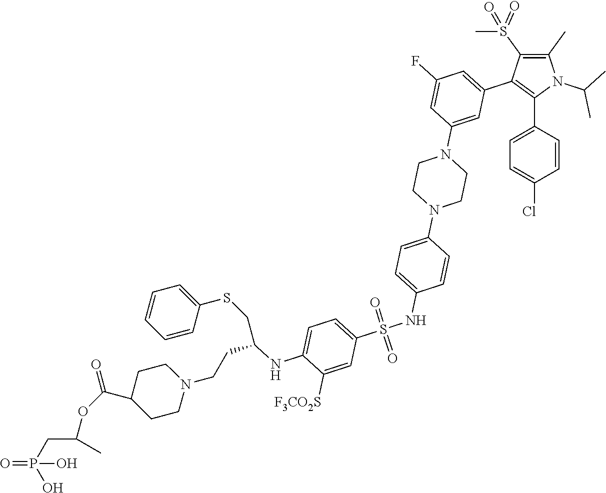 |
| A11 | 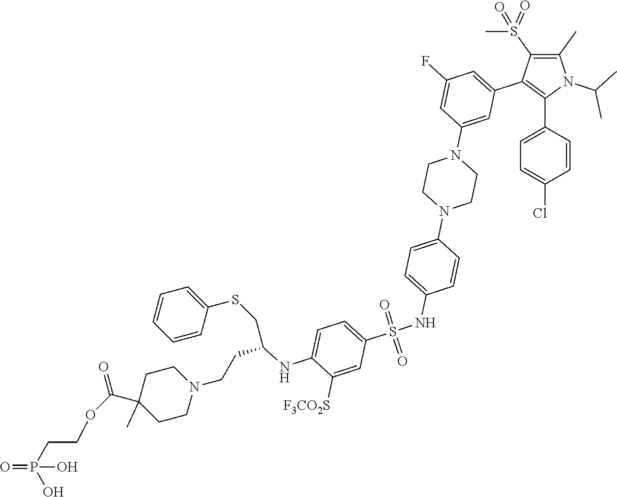 |
| A12 | 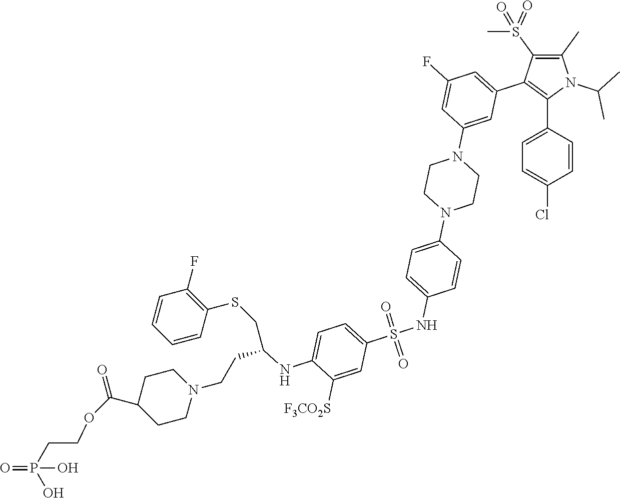 |
| A13 | 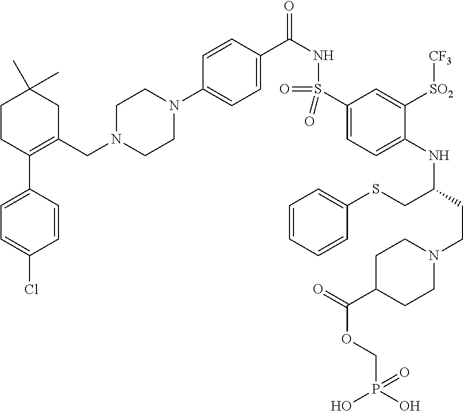 [SMILES: CC1(C)CCC(c2ccc(Cl)cc2)=C(CN2CCN(c3ccc(C(=O)NS(=O)(=O)c4ccc(N[C@H](CCN5CCC(C(=O)OCP(=O)(O)O)CC5)CSc5ccccc5)c(S(=O)(=O)C(F)(F)F)c4)cc3)CC2)C1] |
| A14 | 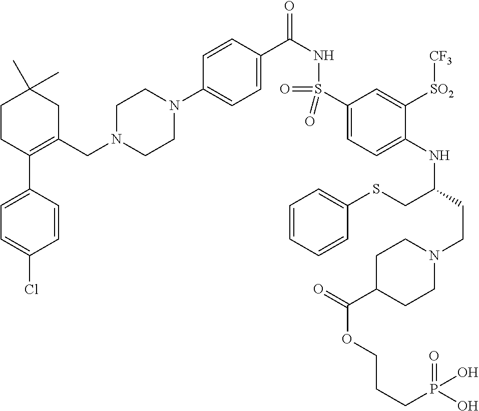 [SMILES: CC1(C)CCC(c2ccc(Cl)cc2)=C(CN2CCN(c3ccc(C(=O)NS(=O)(=O)c4ccc(N[C@H](CCN5CCC(C(=O)OCCCP(=O)(O)O)CC5)CSc5ccccc5)c(S(=O)(=O)C(F)(F)F)c4)cc3)CC2)C1] |
| A15 | 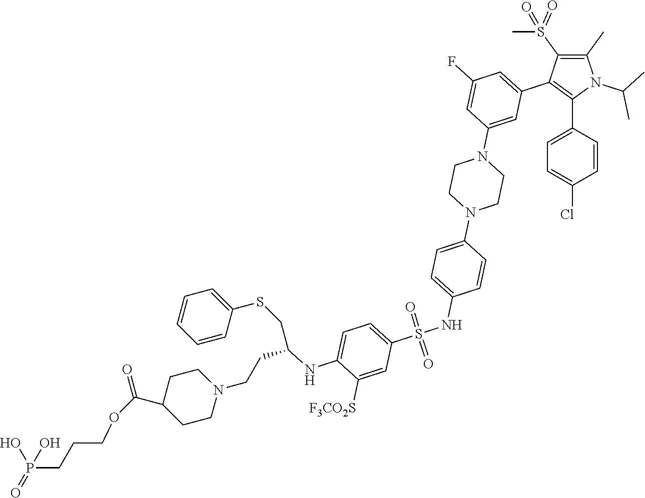 |
| A16 | 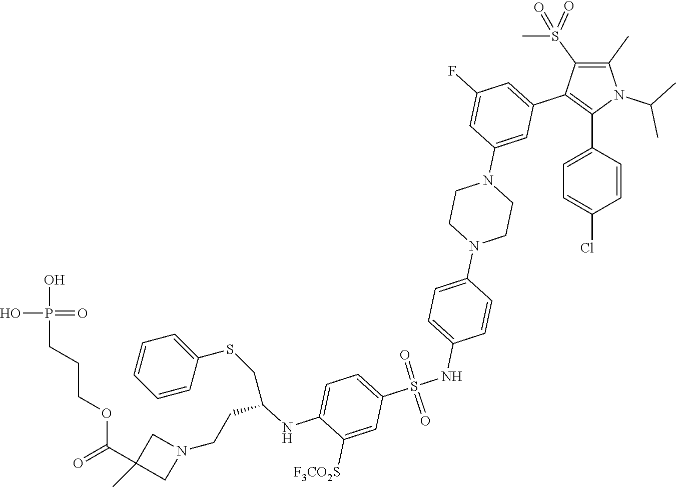 |
| A17 | 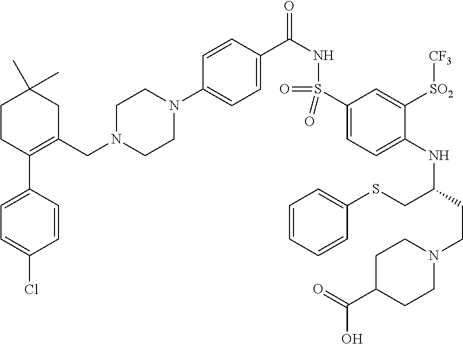 [SMILES: CC1(C)CCC(c2ccc(Cl)cc2)=C(CN2CCN(c3ccc(C(=O)NS(=O)(=O)c4ccc(N[C@H](CCN5CCC(C(=O)O)CC5)CSc5ccccc5)c(S(=O)(=O)C(F)(F)F)c4)cc3)CC2)C1] |
| A18 | 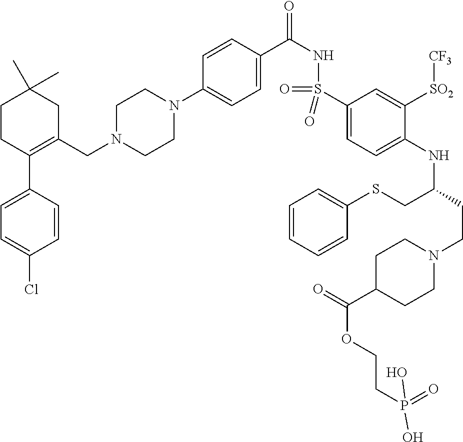 [SMILES: CC1(C)CCC(c2ccc(Cl)cc2)=C(CN2CCN(c3ccc(C(=O)NS(=O)(=O)c4ccc(N[C@H](CCN5CCC(C(=O)OCCP(=O)(O)O)CC5)CSc5ccccc5)c(S(=O)(=O)C(F)(F)F)c4)cc3)CC2)C1] |
| A19 | 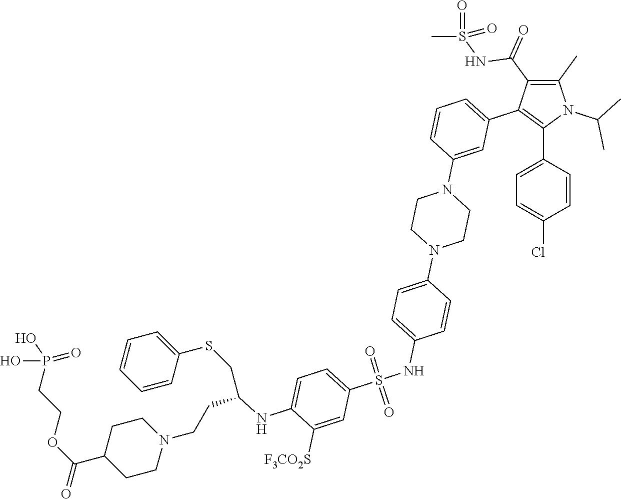 |

In certain embodiments, the Bcl-2/Bcl-xL dual inhibitors described herein having a structural formula (IV) are selected from Table 1B

TABLE 1B
Exemplary compounds of Formula (IV)

| Compound | Structure |
|---|---|
| B1 | 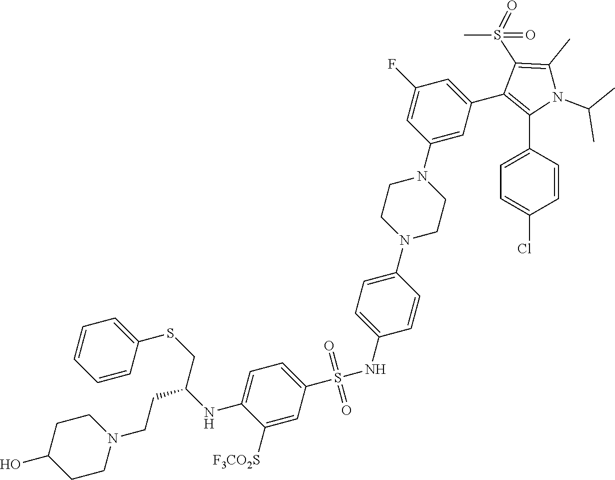 |
| B2 | 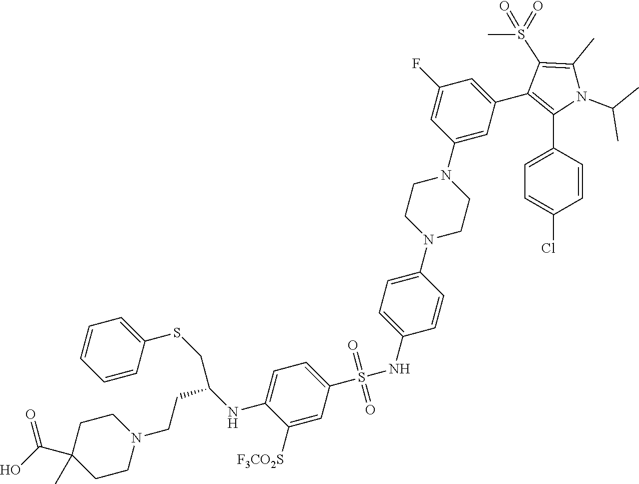 |
| B3 | 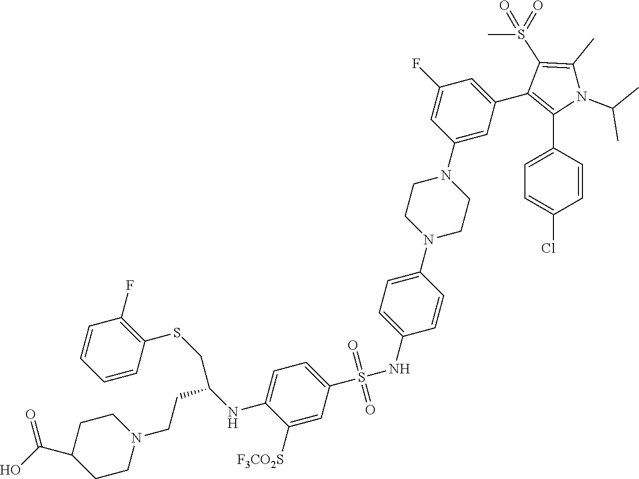 |
| B4 | 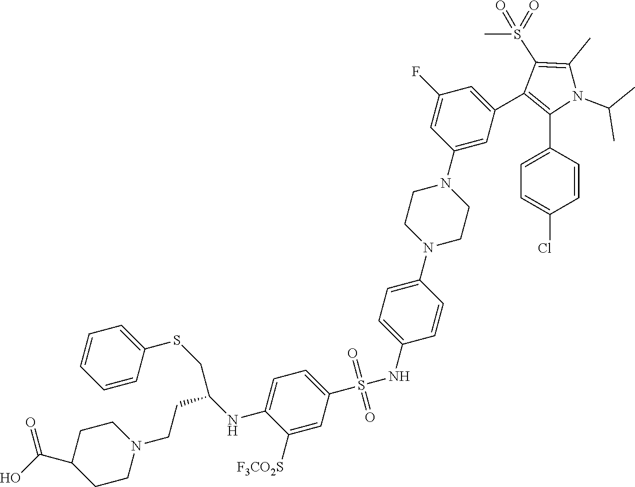 |
| B5 | 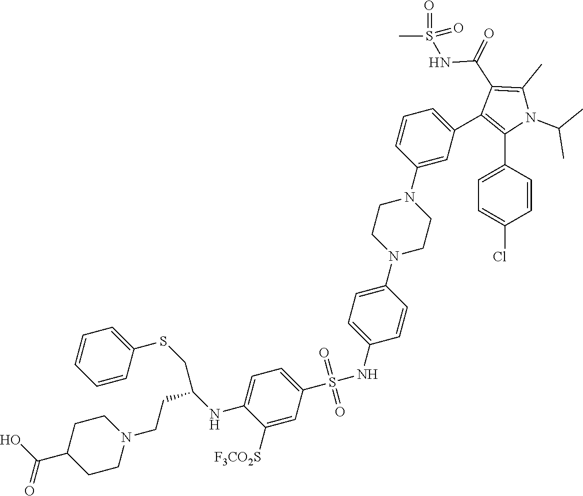 |
| B6 | 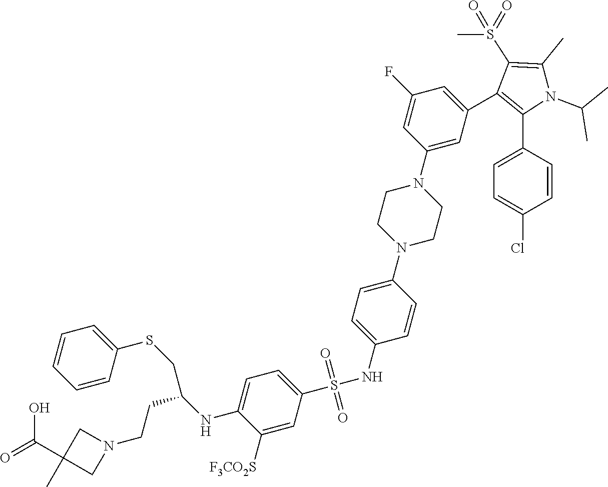 |
| B7 | 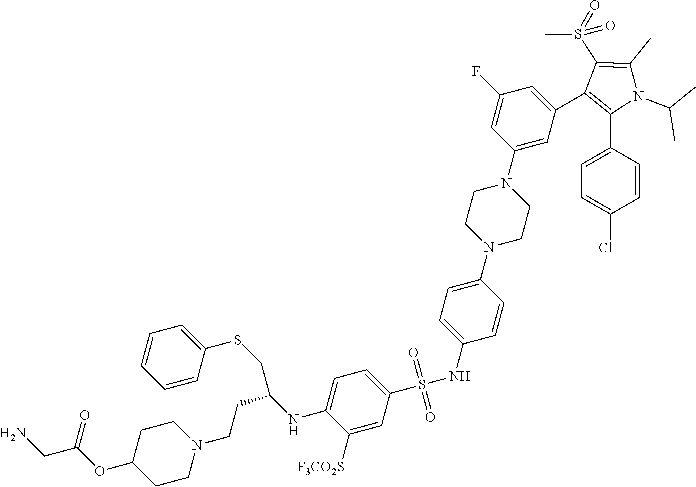 |
| B8 | 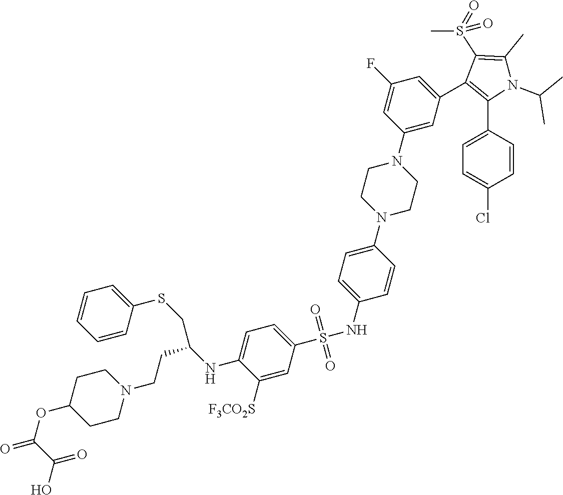 |
| B9 | 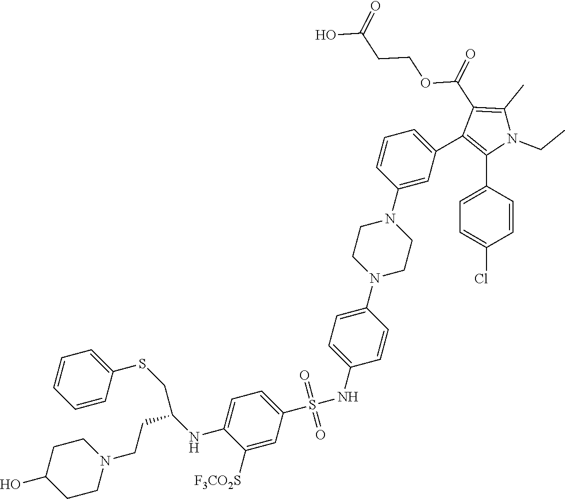 |
| B10 | 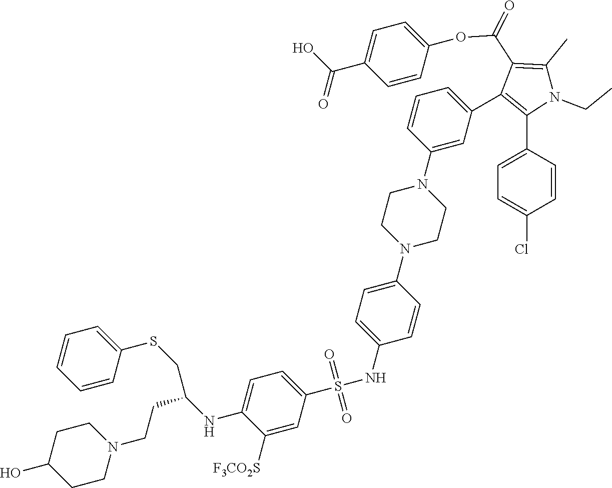 |
| B11 | 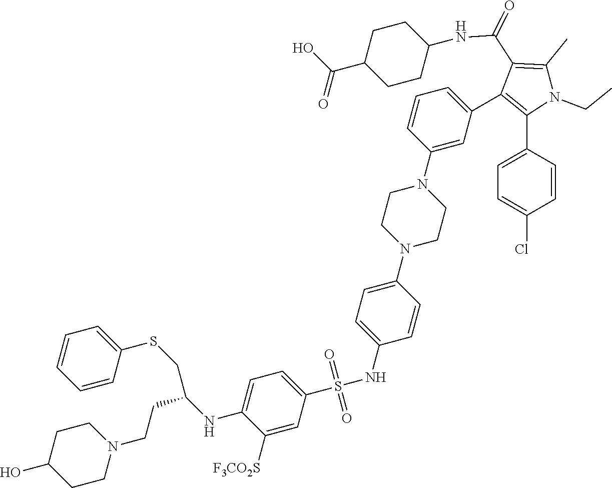 |
| B12 | 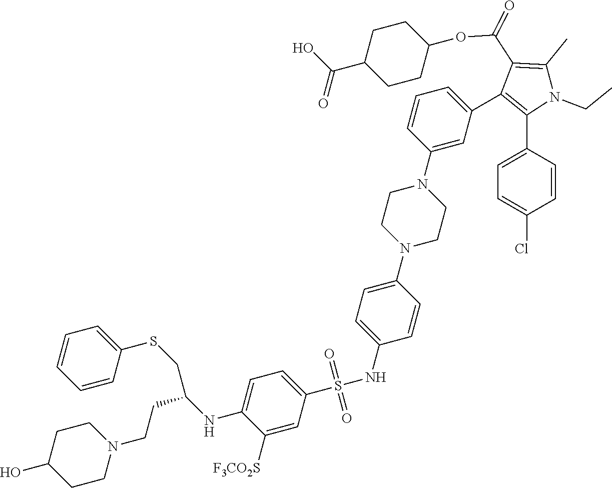 |

In certain embodiments, the Bcl-2 inhibitors described herein having a structural formula (V) are selected from Table 1C.

TABLE 1C
Exemplary compounds of Formula (V)

| Compound | Structure |
|---|---|
| C1 | 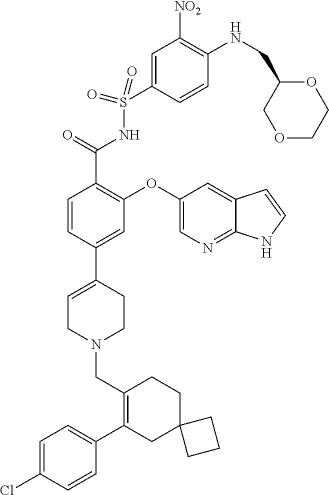 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CCC4)CC3)CC2)cc1Oc1cnc2[nH]ccc2c1] |
| C2 | 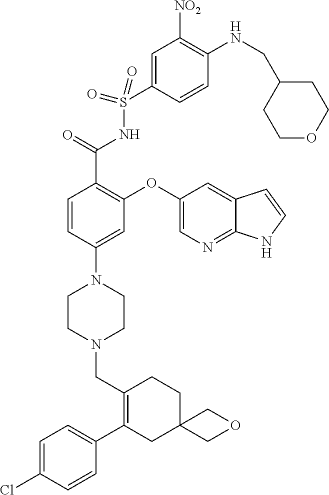 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1] |
| C3 | 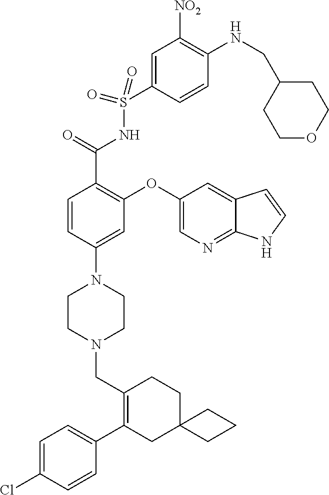 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C4 | 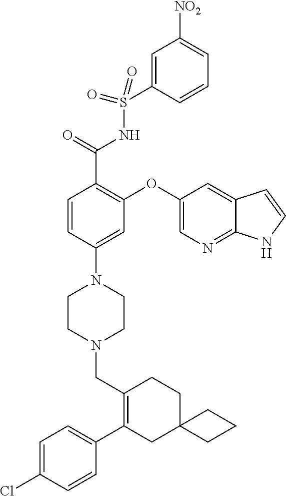 [SMILES: O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1] |
| C5 | 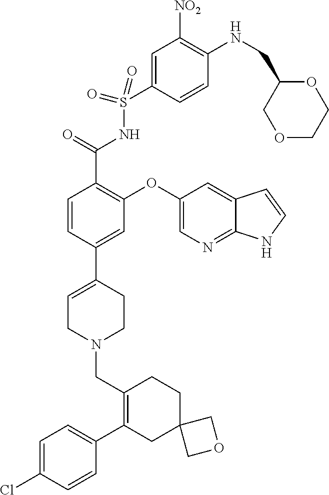 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CC3)COC4)CC2)cc1Oc1cnc2[nH]ccc2c1] |
| C6 | 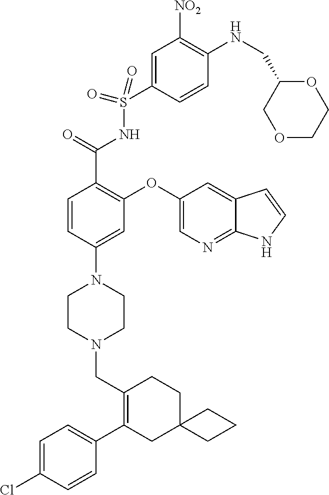 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C7 | 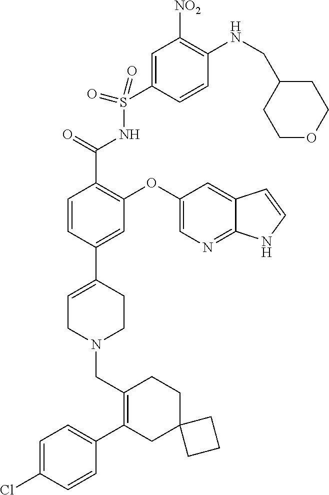 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CCC4)CC3)CC2)cc1Oc1cnc2[nH]ccc2c1] |
| C8 | 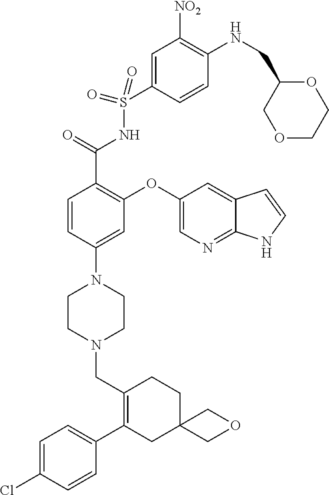 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1] |
| C9 | 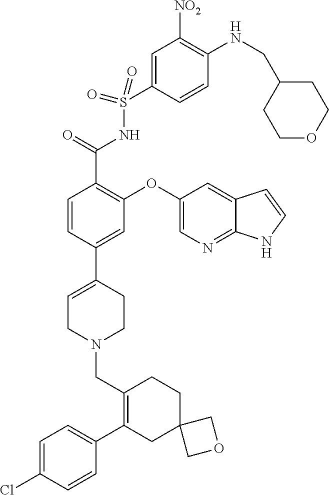 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1] |
| C10 | 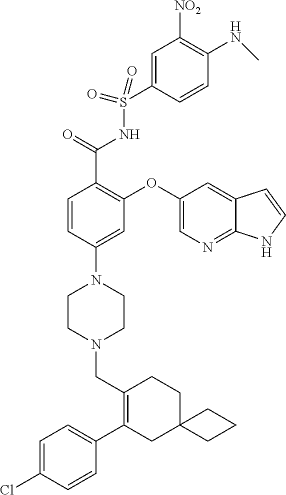 [SMILES: CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] |
| C11 | 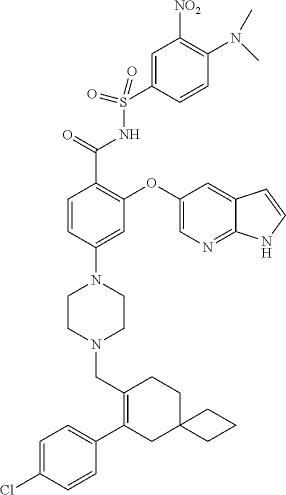 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] |
| C12 | 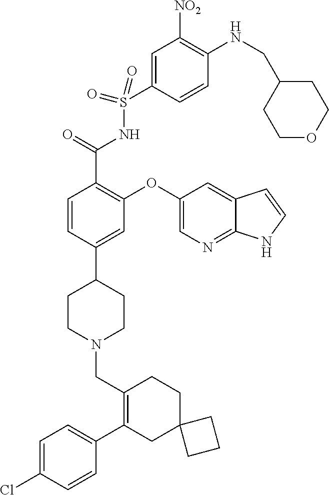 [SMILES: O=C(NS(=O)(=O)c1ccc(NCC2CCOCC2)c([N+](=O)[O-])c1)c1ccc(C2CCN(CC3=C(c4ccc(Cl)cc4)CC4(CCC4)CC3)CC2)cc1Oc1cnc2[nH]ccc2c1] |
| C13 | 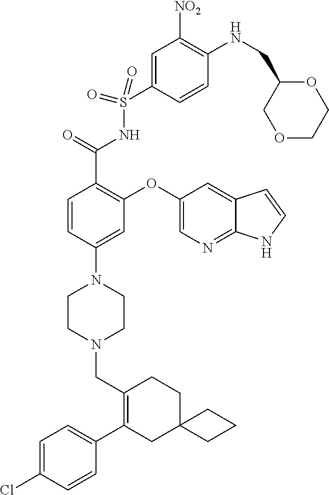 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C14 | 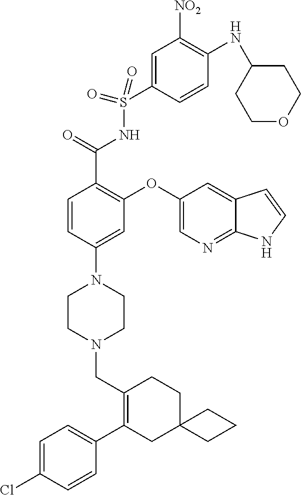 [SMILES: O=C(NS(=O)(=O)c1ccc(NC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C15 | 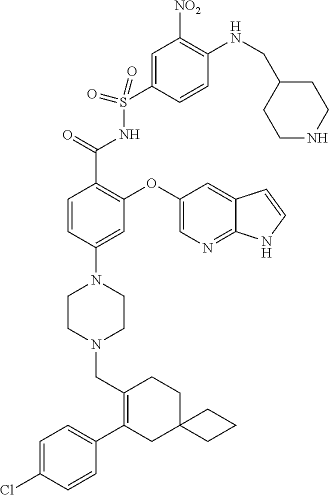 [SMILES: O=C(NS(=O)(=O)c1ccc(NCC2CCNCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C16 | 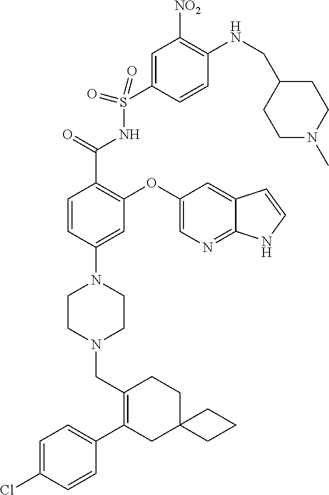 [SMILES: CN1CCC(CNc2ccc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2[N+](=O)[O-])CC1] |
| C17 | 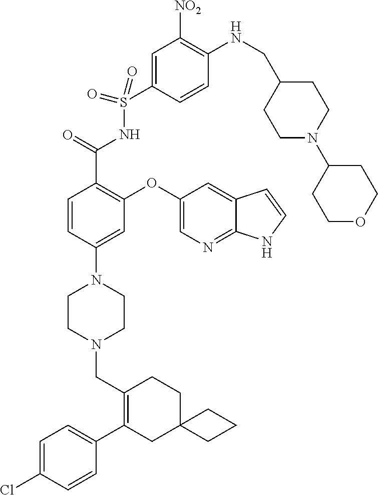 [SMILES: O=C(NS(=O)(=O)c1ccc(NCC2CCN(C3CCOCC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C18 | 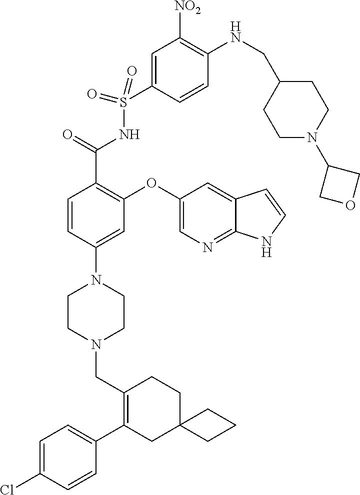 [SMILES: O=C(NS(=O)(=O)c1ccc(NCC2CCN(C3COC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C19 | 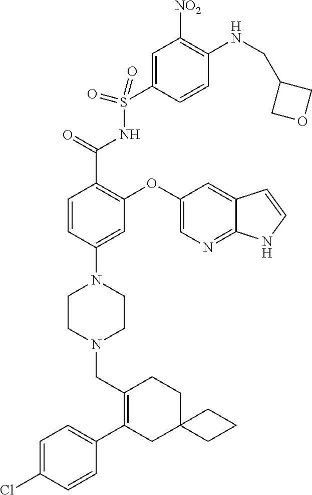 [SMILES: O=C(NS(=O)(=O)c1ccc(NCC2COC2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C20 | 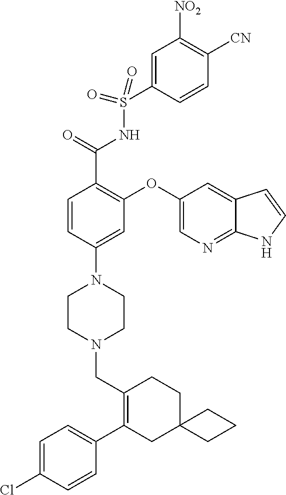 [SMILES: N#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] |
| C21 | 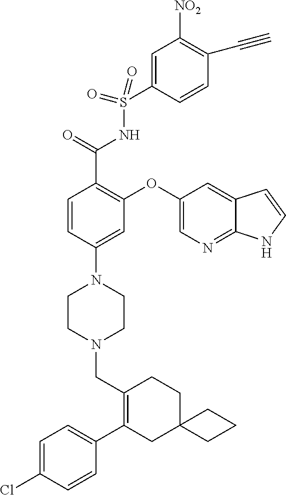 [SMILES: C#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] |
| C22 | 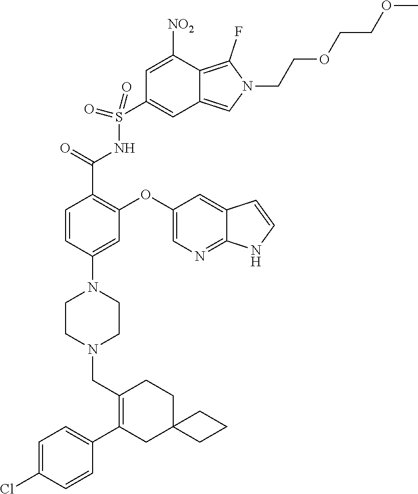 [SMILES: COCCOCCn1cc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2c1F] |
| C23 | 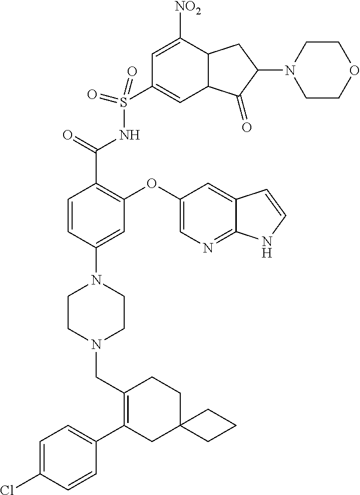 [SMILES: O=C(NS(=O)(=O)C1=CC2C(=O)C(N3CCOCC3)CC2C([N+](=O)[O-])=C1)c1ccc(N2CCN(CC3=C(c4ccc(Cl)cc4)CC4(CCC4)CC3)CC2)cc1Oc1cnc2[nH]ccc2c1] |
| C24 | 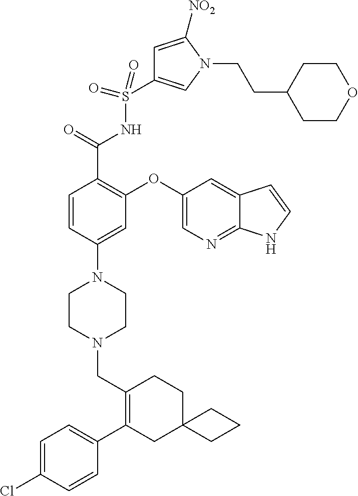 [SMILES: O=C(NS(=O)(=O)c1cc([N+](=O)[O-])n(CCC2CCOCC2)c1)c1ccc(N2CCN(CC3=C(c4ccc(Cl)cc4)CC4(CCC4)CC3)CC2)cc1Oc1cnc2[nH]ccc2c1] |
| C25 | 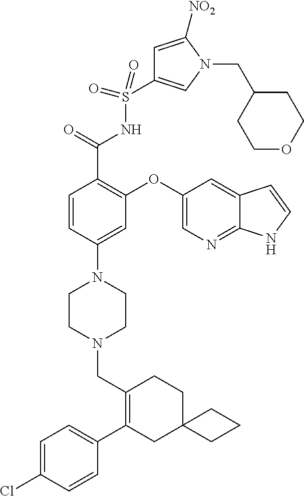 [SMILES: O=C(NS(=O)(=O)c1cc([N+](=O)[O-])n(CC2CCOCC2)c1)c1ccc(N2CCN(CC3=C(c4ccc(Cl)cc4)CC4(CCC4)CC3)CC2)cc1Oc1cnc2[nH]ccc2c1] |
| C26 | 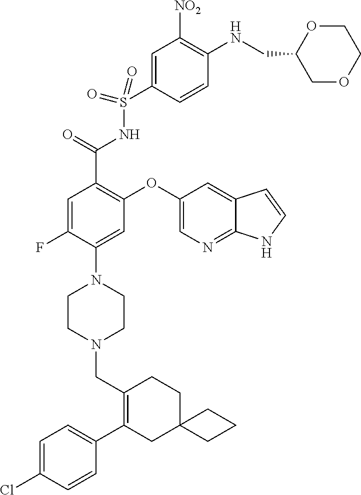 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] |
| C27 | 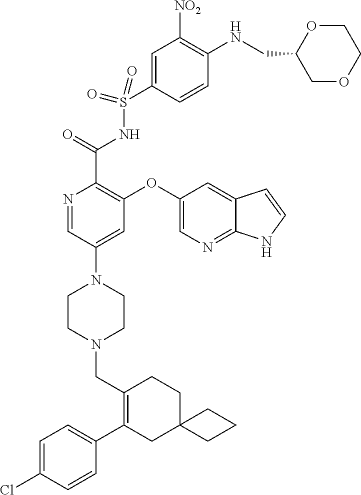 [SMILES: O=C(NS(=O)(=O)c1ccc(NC[C@H]2COCCO2)c([N+](=O)[O-])c1)c1ncc(N2CCN(CC3=C(c4ccc(Cl)cc4)CC4(CCC4)CC3)CC2)cc1Oc1cnc2[nH]ccc2c1] |
| C28 | 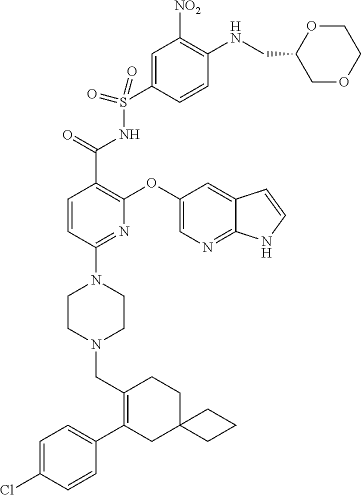 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)nc1Oc1cnc2[nH]ccc2c1] |
| C29 | 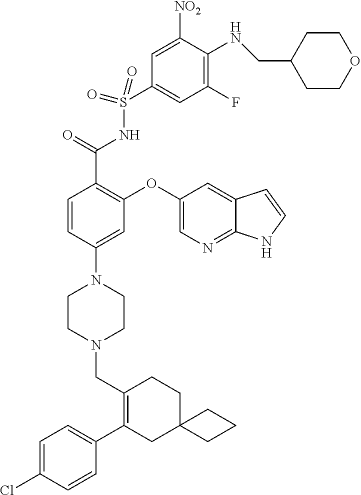 [SMILES: O=C(NS(=O)(=O)c1cc(F)c(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C30 | 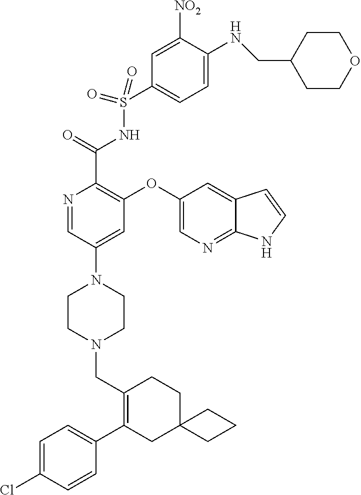 [SMILES: O=C(NS(=O)(=O)c1ccc(NCC2CCOCC2)c([N+](=O)[O-])c1)c1ncc(N2CCN(CC3=C(c4ccc(Cl)cc4)CC4(CCC4)CC3)CC2)cc1Oc1cnc2[nH]ccc2c1] |
| C31 | 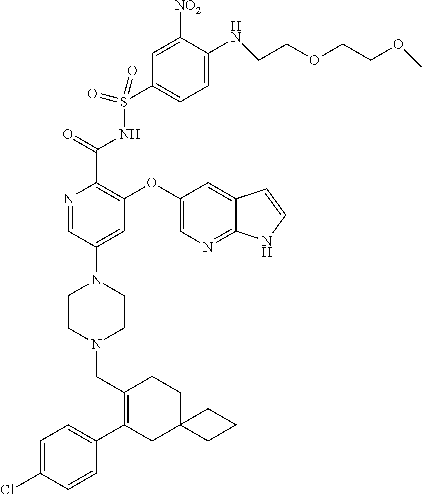 [SMILES: COCCOCCNc1ccc(S(=O)(=O)NC(=O)c2ncc(N3CCN(CC4=C(c5ccc(Cl)cc5)CC5(CCC5)CC4)CC3)cc2Oc2cnc3[nH]ccc3c2)cc1[N+](=O)[O-]] |
| C32 | 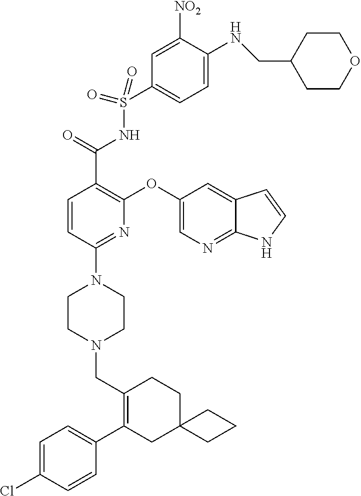 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)nc1Oc1cnc2[nH]ccc2c1] |
| C33 | 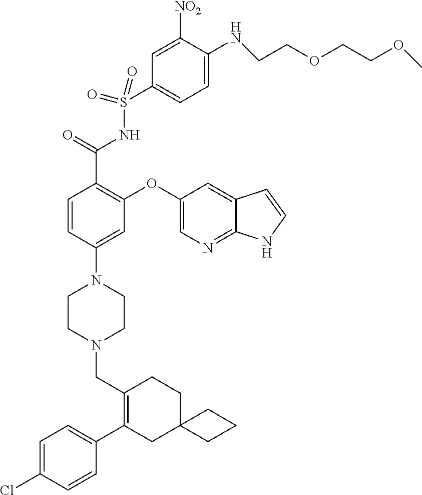 [SMILES: COCCOCCNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] |
| C34 | 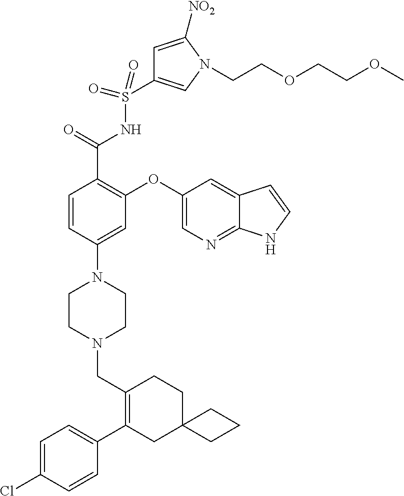 [SMILES: COCCOCCn1cc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] |
| C35 | 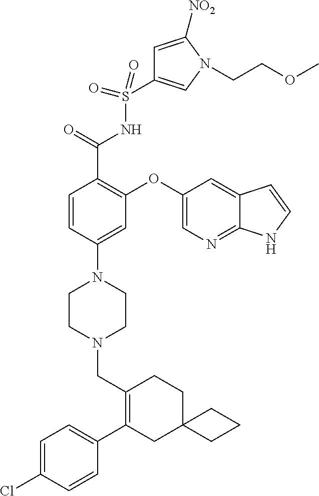 [SMILES: COCCn1cc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] |
| C36 | 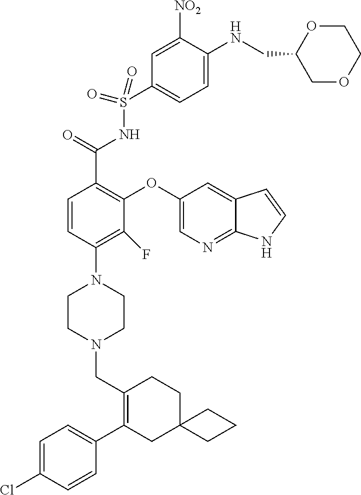 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] |
| C37 | 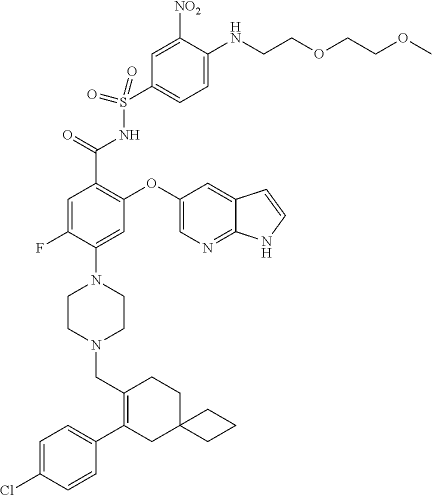 [SMILES: COCCOCCNc1ccc(S(=O)(=O)NC(=O)c2cc(F)c(N3CCN(CC4=C(c5ccc(Cl)cc5)CC5(CCC5)CC4)CC3)cc2Oc2cnc3[nH]ccc3c2)cc1[N+](=O)[O-]] |
| C38 | 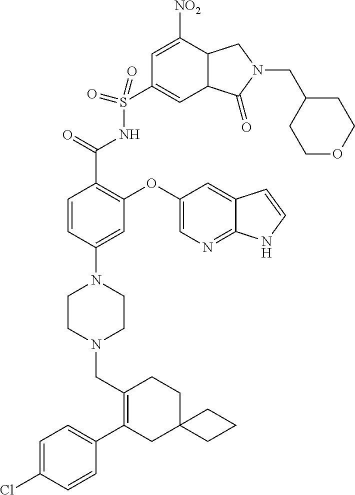 [SMILES: O=C(NS(=O)(=O)C1=CC2C(=O)N(CC3CCOCC3)CC2C([N+](=O)[O-])=C1)c1ccc(N2CCN(CC3=C(c4ccc(Cl)cc4)CC4(CCC4)CC3)CC2)cc1Oc1cnc2[nH]ccc2c1] |
| C39 | 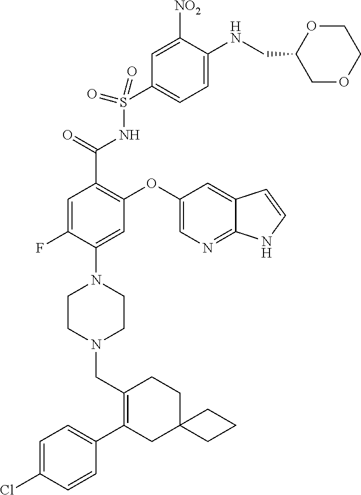 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] |
| C40 | 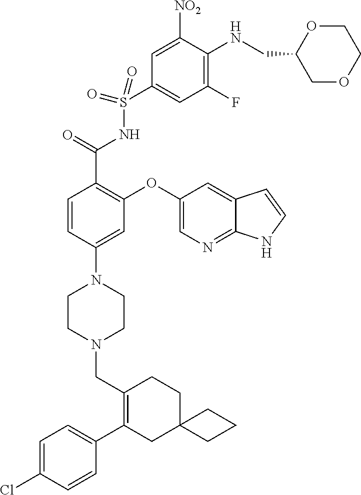 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] |
| C41 | 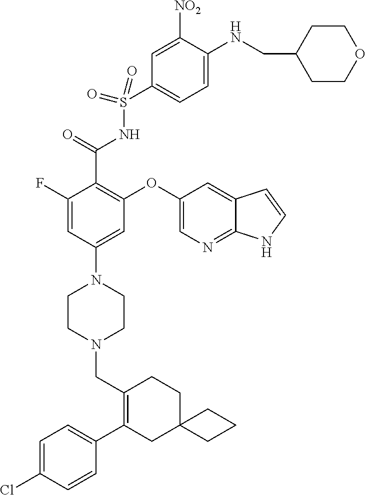 [SMILES: O=C(NS(=O)(=O)c1ccc(NCC2CCOCC2)c([N+](=O)[O-])c1)c1c(F)cc(N2CCN(CC3=C(c4ccc(Cl)cc4)CC4(CCC4)CC3)CC2)cc1Oc1cnc2[nH]ccc2c1] |
| C42 | 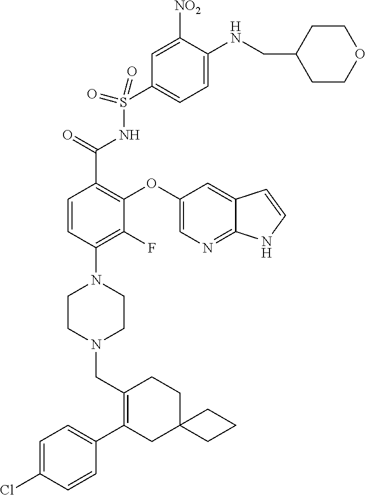 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] |
| C43 | 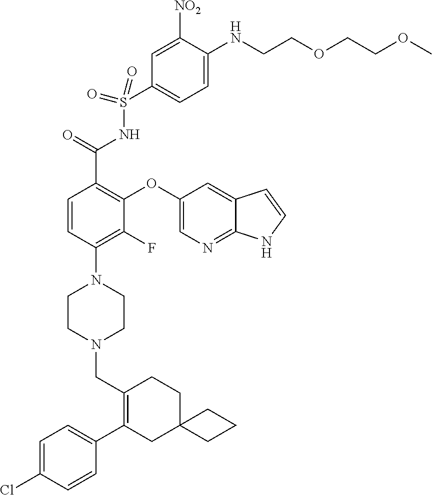 [SMILES: COCCOCCNc1ccc(S(=O)(=O)NC(=O)c2ccc(N3CCN(CC4=C(c5ccc(Cl)cc5)CC5(CCC5)CC4)CC3)c(F)c2Oc2cnc3[nH]ccc3c2)cc1[N+](=O)[O-]] |
| C44 | 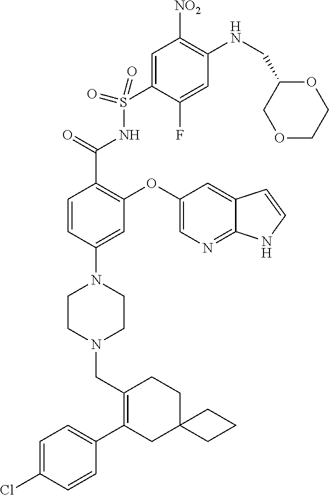 [SMILES: O=C(NS(=O)(=O)c1cc([N+](=O)[O-])c(NC[C@H]2COCCO2)cc1F)c1ccc(N2CCN(CC3=C(c4ccc(Cl)cc4)CC4(CCC4)CC3)CC2)cc1Oc1cnc2[nH]ccc2c1] |
| C45 | 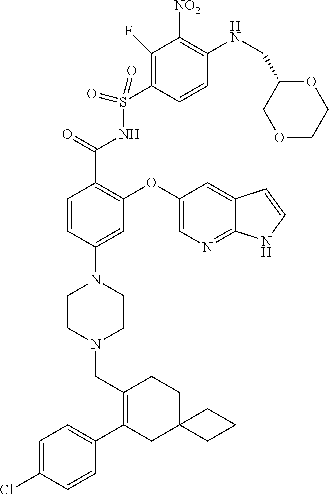 [SMILES: O=C(NS(=O)(=O)c1ccc(NC[C@H]2COCCO2)c([N+](=O)[O-])c1F)c1ccc(N2CCN(CC3=C(c4ccc(Cl)cc4)CC4(CCC4)CC3)CC2)cc1Oc1cnc2[nH]ccc2c1] |
| C46 | 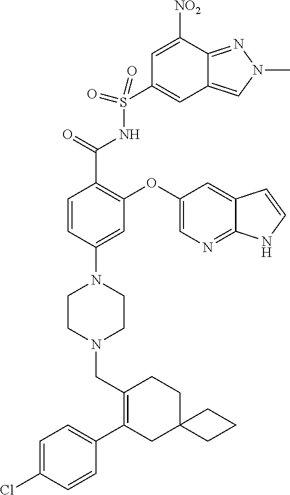 [SMILES: Cn1cc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2n1] |
| C47 | 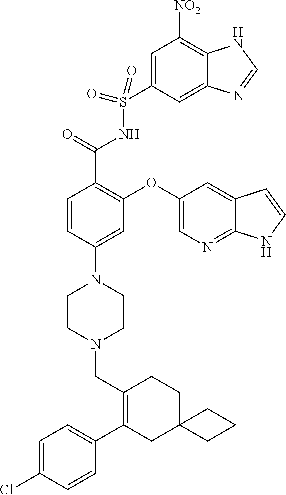 [SMILES: O=C(NS(=O)(=O)c1cc([N+](=O)[O-])c2[nH]cnc2c1)c1ccc(N2CCN(CC3=C(c4ccc(Cl)cc4)CC4(CCC4)CC3)CC2)cc1Oc1cnc2[nH]ccc2c1] |
| C48 | 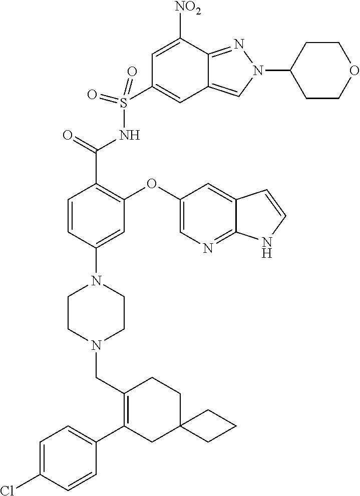 [SMILES: O=C(NS(=O)(=O)c1cc([N+](=O)[O-])c2nn(C3CCOCC3)cc2c1)c1ccc(N2CCN(CC3=C(c4ccc(Cl)cc4)CC4(CCC4)CC3)CC2)cc1Oc1cnc2[nH]ccc2c1] |
| C49 | 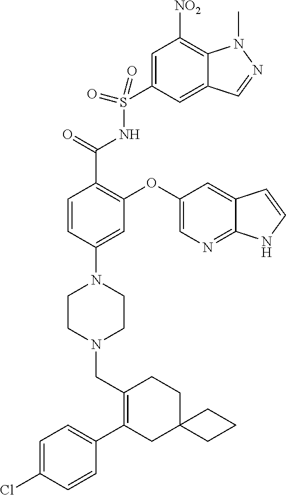 [SMILES: Cn1ncc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c21] |
| C50 | 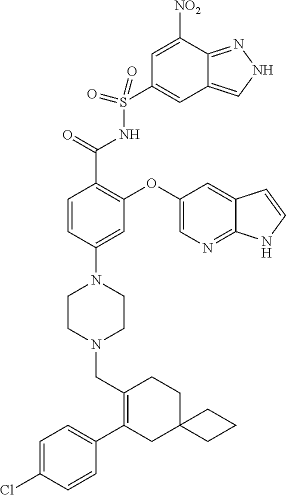 [SMILES: O=C(NS(=O)(=O)c1cc([N+](=O)[O-])c2n[nH]cc2c1)c1ccc(N2CCN(CC3=C(c4ccc(Cl)cc4)CC4(CCC4)CC3)CC2)cc1Oc1cnc2[nH]ccc2c1] |
| C51 | 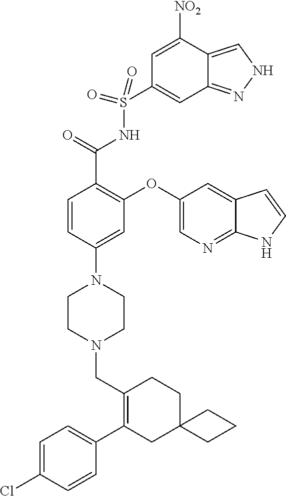 [SMILES: O=C(NS(=O)(=O)c1cc([N+](=O)[O-])c2c[nH]nc2c1)c1ccc(N2CCN(CC3=C(c4ccc(Cl)cc4)CC4(CCC4)CC3)CC2)cc1Oc1cnc2[nH]ccc2c1] |
| C52 | 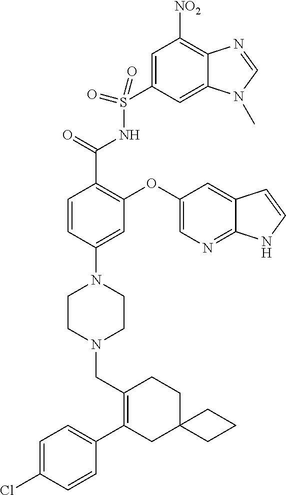 [SMILES: Cn1cnc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc21] |
| C53 | 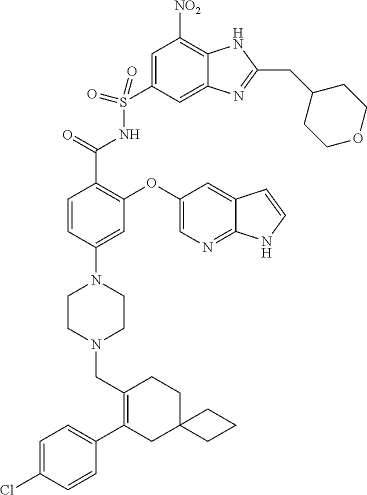 [SMILES: O=C(NS(=O)(=O)c1cc([N+](=O)[O-])c2[nH]c(CC3CCOCC3)nc2c1)c1ccc(N2CCN(CC3=C(c4ccc(Cl)cc4)CC4(CCC4)CC3)CC2)cc1Oc1cnc2[nH]ccc2c1] |
| C54 | 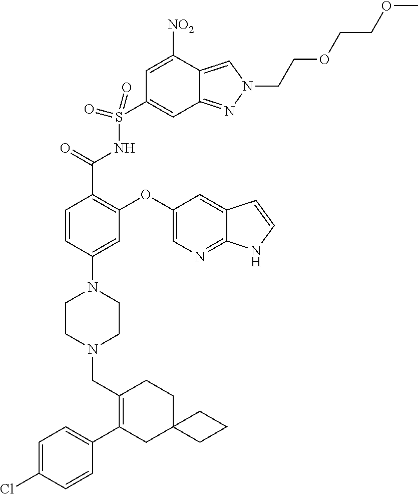 [SMILES: COCCOCCn1cc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2n1] |
| C55 | 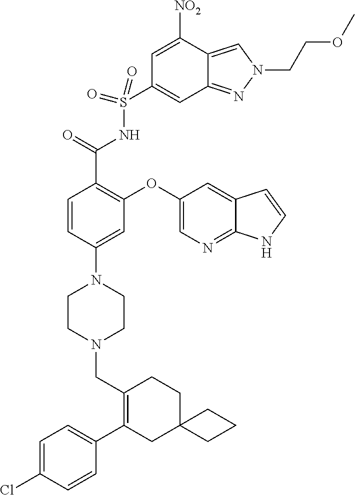 [SMILES: COCCn1cc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2n1] |
| C56 | 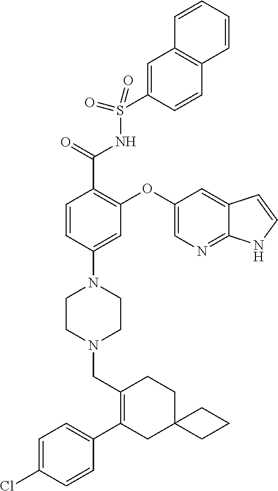 [SMILES: O=C(NS(=O)(=O)c1ccc2ccccc2c1)c1ccc(N2CCN(CC3=C(c4ccc(Cl)cc4)CC4(CCC4)CC3)CC2)cc1Oc1cnc2[nH]ccc2c1] |
| C57 | 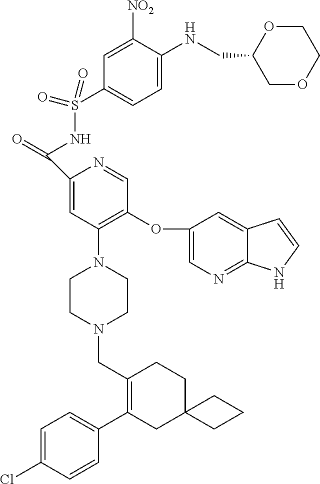 [SMILES: O=C(NS(=O)(=O)c1ccc(NC[C@H]2COCCO2)c([N+](=O)[O-])c1)c1cc(N2CCN(CC3=C(c4ccc(Cl)cc4)CC4(CCC4)CC3)CC2)c(Oc2cnc3[nH]ccc3c2)cn1] |
| C58 | 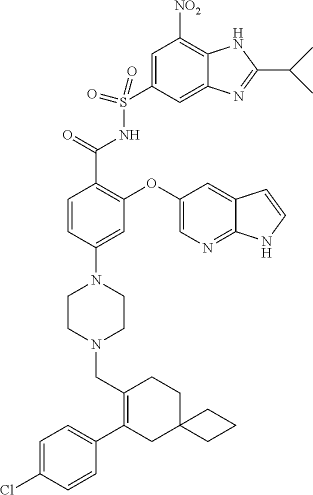 [SMILES: CC(C)c1nc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2[nH]1] |
| C59 | 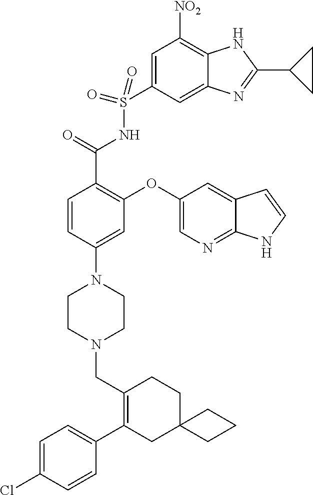 [SMILES: O=C(NS(=O)(=O)c1cc([N+](=O)[O-])c2[nH]c(C3CC3)nc2c1)c1ccc(N2CCN(CC3=C(c4ccc(Cl)cc4)CC4(CCC4)CC3)CC2)cc1Oc1cnc2[nH]ccc2c1] |
| C60 | 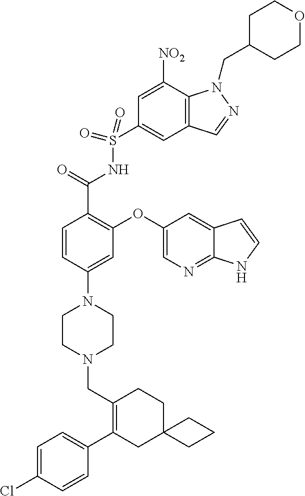 [SMILES: O=C(NS(=O)(=O)c1cc([N+](=O)[O-])c2c(cnn2CC2CCOCC2)c1)c1ccc(N2CCN(CC3=C(c4ccc(Cl)cc4)CC4(CCC4)CC3)CC2)cc1Oc1cnc2[nH]ccc2c1] |
| C61 | 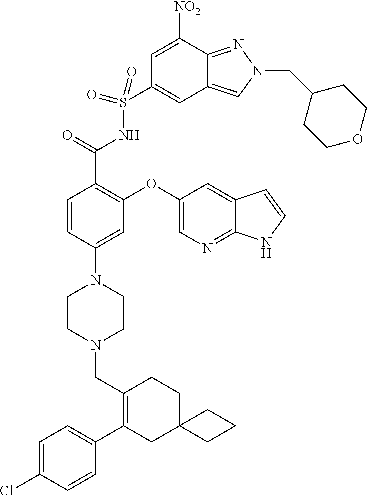 [SMILES: O=C(NS(=O)(=O)c1cc([N+](=O)[O-])c2nn(CC3CCOCC3)cc2c1)c1ccc(N2CCN(CC3=C(c4ccc(Cl)cc4)CC4(CCC4)CC3)CC2)cc1Oc1cnc2[nH]ccc2c1] |
| C62 | 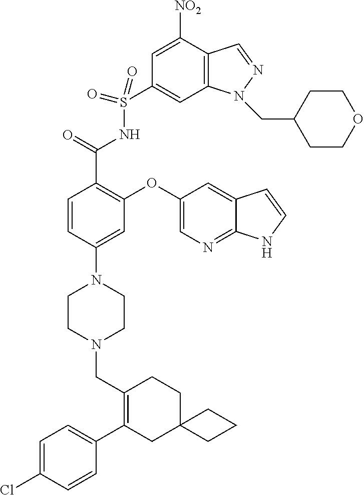 [SMILES: O=C(NS(=O)(=O)c1cc([N+](=O)[O-])c2cnn(CC3CCOCC3)c2c1)c1ccc(N2CCN(CC3=C(c4ccc(Cl)cc4)CC4(CCC4)CC3)CC2)cc1Oc1cnc2[nH]ccc2c1] |
| C63 | 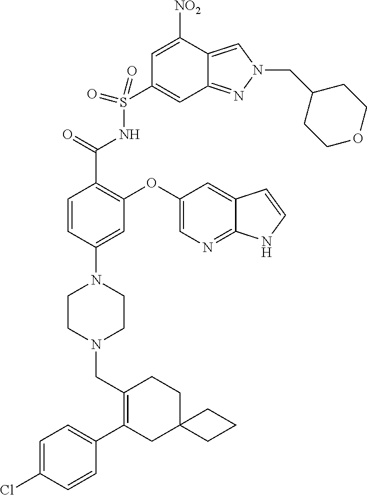 [SMILES: O=C(NS(=O)(=O)c1cc([N+](=O)[O-])c2cn(CC3CCOCC3)nc2c1)c1ccc(N2CCN(CC3=C(c4ccc(Cl)cc4)CC4(CCC4)CC3)CC2)cc1Oc1cnc2[nH]ccc2c1] |
| C64 | 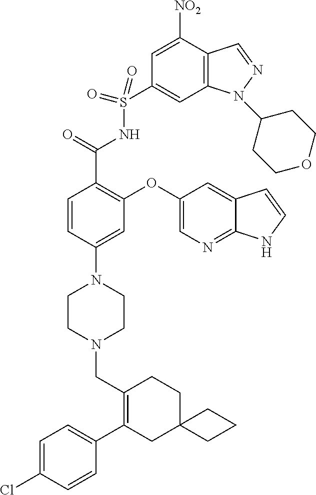 [SMILES: O=C(NS(=O)(=O)c1cc([N+](=O)[O-])c2cnn(C3CCOCC3)c2c1)c1ccc(N2CCN(CC3=C(c4ccc(Cl)cc4)CC4(CCC4)CC3)CC2)cc1Oc1cnc2[nH]ccc2c1] |
| C65 | 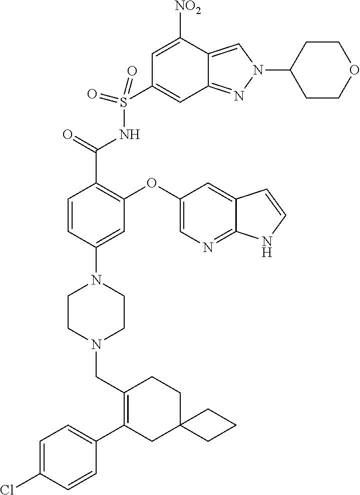 [SMILES: O=C(NS(=O)(=O)c1cc([N+](=O)[O-])c2cn(C3CCOCC3)nc2c1)c1ccc(N2CCN(CC3=C(c4ccc(Cl)cc4)CC4(CCC4)CC3)CC2)cc1Oc1cnc2[nH]ccc2c1] |
| C66 | 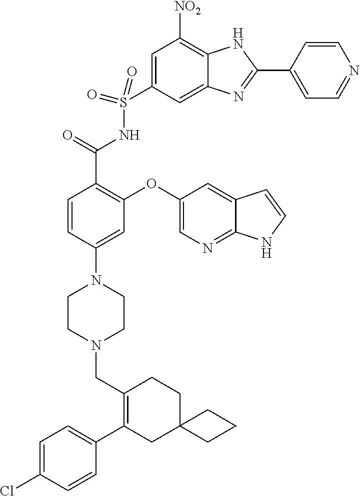 [SMILES: O=C(NS(=O)(=O)c1cc([N+](=O)[O-])c2[nH]c(-c3ccncc3)nc2c1)c1ccc(N2CCN(CC3=C(c4ccc(Cl)cc4)CC4(CCC4)CC3)CC2)cc1Oc1cnc2[nH]ccc2c1] |

In one embodiment, the Bcl-2 inhibitor is 4-{4-{[6-(4-Chlorophenyl)spiro[3.5]non-6-en-7-yl]methyl}-piperazin-1-yl}-N-{{3-nitro-4-[((2S)-1,4-dioxan-2-ylmethyl)amino]phenyl}sulfonyl}-2-(1H-pyrrolo[2,3-b]pyridin-5-yloxy)benzamide or a pharmaceutically acceptable salt thereof. This compound is also referred to herein as Compound C6 having the following structure:

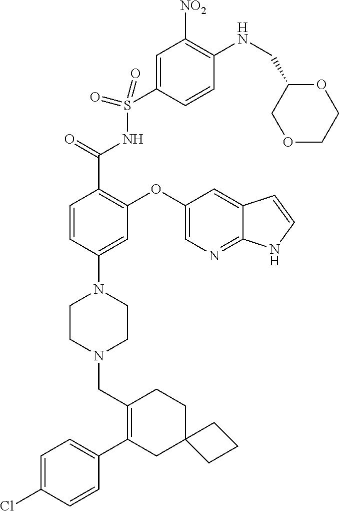 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In certain embodiments, the Bcl-2/Bcl-xL dual inhibitor is (R)-2-(1-(3-(4-(N-(4-(4-(3-(2-(4-chlorophenyl)-1-isopropyl-5-methyl-4-(methylsulfonyl)-1H-pyrrol-3-yl)-5-fluorophenyl)piperazin-1-yl)phenyl)sulfamoyl)-2-(trifluoromethylsulfonyl)phenylamino)-4-(phenylthio)butyl)piperidine-4-carbonyloxy)ethylphosphonic acid (“Compound A15”) having the following structure

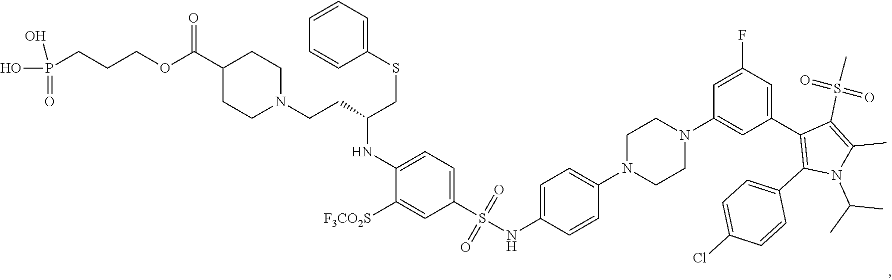

or a pharmaceutically acceptable salt thereof.

In certain embodiments, the Bcl-2/Bcl-xL dual inhibitor is (R)-1-(3-(4-(N-(4-(4-(3-(2-(4-chlorophenyl)-1-isopropyl-5-methyl-4-(methyl sulfonyl)-1H-pyrrol-3-yl)-5-fluorophenyl) piperazin-1-yl)phenyl) sulfamoyl)-2-(trifluoromethylsulfonyl)phenyl amino)-4-(phenylthio)butyl)piperidine-4-carboxylic acid (“Compound B4”) having the following structure

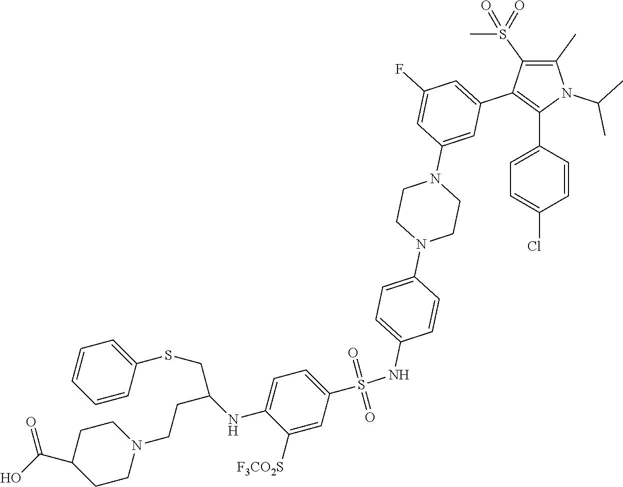

or a pharmaceutically acceptable salt thereof.

Compound A15 is a small-molecule compound that binds to Bcl-2, Bcl-xL and Bcl-w proteins with very high affinities with IC₅₀ values of 1.6 nM, 4.4 nM, and 9.3 nM, respectively. Compound A15 has a weak affinity to Mcl-1 protein. Compound A15 demonstrates potent cell growth inhibitory activity in vitro with nanomolar potencies in a subset of cancer cell lines. Mechanistically, Compound A15 effectively induces cleavage of caspase-3 and PARP, biochemical markers of apoptosis of human cancers in cancer cells and in xenograft tumor tissues. Compound B4 is an active metabolite of Compound A15.

More Bcl-2/Bcl-xL inhibitors and the synthesis thereof are further disclosed in WO2014/113413A1, PCT/CN2019/070508, and WO 2018/027097 A1, which are incorporated herein by reference.

In some embodiments, the method for treating T-cell prolymphocytic leukemia (T-PLL) comprises administering an effective amount of the MDM2 inhibitor having the following formula (Compound C) or a pharmaceutically acceptable salt thereof:

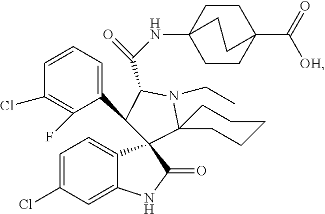 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

and
an effective amount of the Bcl-2 inhibitor of the following formula (Compound C6), or a pharmaceutically acceptable salt thereof

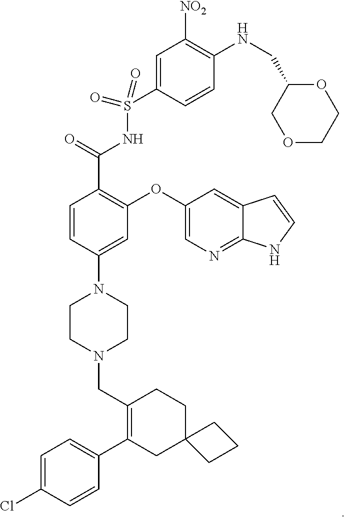 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In some embodiments, the method for treating T-cell prolymphocytic leukemia (T-PLL) comprises administering an effective amount of the MDM2 inhibitor having the following formula (i.e. Compound C) or a pharmaceutically acceptable salt thereof:

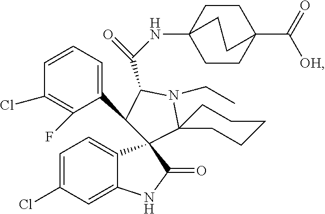 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

and
an effective amount of the Bcl-2/Bcl-xL inhibitor selected from Compound A15 (chemical formula shown in Table 1A and also below) and Compound B4 (chemical formulas shown in Table 1B and also below), or a pharmaceutically acceptable salt thereof:

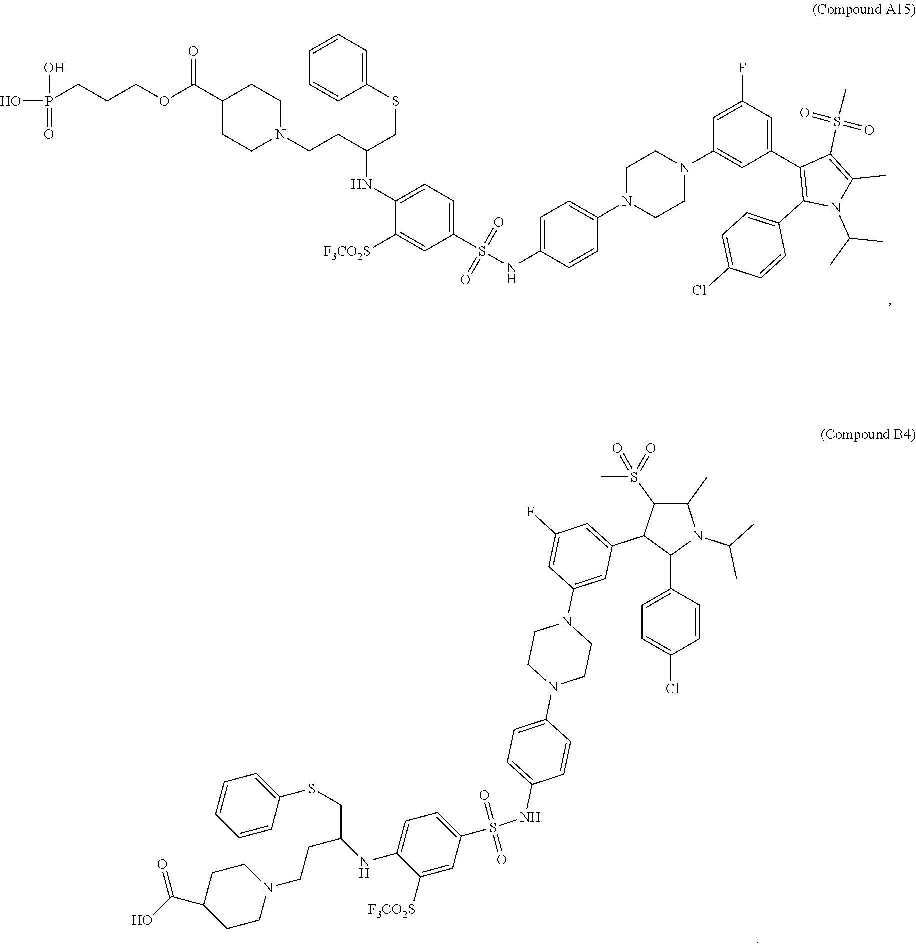

### III. Methods of Treating Cancer with MDM2 Inhibitors and Immunotherapy

In another aspect, the present disclosure provides methods of treating a subject having cancer, comprising administering to the subject an effective amount of a MDM2 inhibitor and an effective amount of an immunotherapy for example at least one modulator of an immune checkpoint molecule.

The MDM2 inhibitor can be any MDM2 inhibitor as provided and described herein.

### a) Immunotherapies

As used herein, the term “immunotherapy” refers to treatment of diseases by activating or suppressing the immune system. Several types of immunotherapy are used to treat cancer, including but not limited to modulators of immune checkpoints, chimeric antigen receptor (CAR) T-cell therapy, monoclonal antibodies and cancer vaccines.

As used herein, an “immune checkpoint” or “immune checkpoint molecule” is a molecule in the immune system that modulates a signal. An immune checkpoint molecule can be a co-stimulatory checkpoint molecule, i.e., turn up a signal, or an inhibitory checkpoint molecule, i.e., turn down a signal. A “co-stimulatory checkpoint molecule” as used herein is a molecule in the immune system that turns up a signal or is co-stimulatory. An “inhibitory checkpoint molecule”, as used herein is a molecule in the immune system that turns down a signal or is co-inhibitory.

As used herein, a “modulator of an immune checkpoint molecule” is an agent capable of altering the activity of an immune checkpoint in a subject.

The ability of tumor cells to harness a range of complex, overlapping mechanisms to prevent the immune system from distinguishing self from non-self represents the fundamental mechanism of tumors to evade immunesurveillance. Mechanism(s) include disruption of antigen presentation, disruption of regulatory pathways controlling T cell activation or inhibition (immune checkpoint regulation), recruitment of cells that contribute to immune suppression (Tregs, MDSC) or release of factors that influence immune activity (DO, PGE2). See Harris et al., 2013, J Immunotherapy Cancer 1:12; Chen et al., 2013, Immunity 39:1; Pardoll, et al., 2012, Nature Reviews: Cancer 12:252; and Sharma et al., 2015, Cell 161:205, each of which is incorporated by reference herein in its entirety. Recent years have seen an explosion of immune-oncology therapeutic modalities with approaches ranging from inhibitors of T cell checkpoint, T cell activating agents, and potential vaccines either approved for clinical use or under active investigation. A few of these, including anti-CTLA-4, anti-PD-1, and anti-PD-L1 immune checkpoint therapies, have demonstrated variable success and have been approved for clinical use. Although the checkpoint inhibitors are the most advanced in clinical development for treatment of various cancers, these represent a fraction of the potential targets and pathways that can be harnessed to improve anti-tumor responses. This is evidenced by the continuous emergence of new lists of potential molecules influencing checkpoint or inhibitory pathways along with co-stimulatory molecules that improve immune responses that are in various stages of pre-clinical and clinical development. Examples of new immune checkpoints that are being evaluated for cancer treatment include LAG-3 (Triebel et al., 1990, J. Exp. Med. 171: 1393-1405), TIM-3 (Sakuishi et al., 2010, J. Exp. Med. 207: 2187-2194) and VISTA (Wang et al., 2011, J. Exp. Med. 208: 577-592). Examples of co-stimulatory molecules that improve immune responses include ICOS (Fan et al., 2014, J. Exp. Med. 211: 715-725), OX40 (Curti et al., 2013, Cancer Res. 73: 7189-7198) and 4-1BB (Melero et al., 1997, Nat. Med. 3: 682-685).

In some embodiments, the immune checkpoint molecule of the invention is a co-stimulatory immune checkpoint (i.e., a molecule that stimulates the immune response), and the immune checkpoint modulator is an activator (an agonist) of a stimulatory immune checkpoint. In some embodiments, the immune checkpoint molecule of the invention is an inhibitory immune checkpoint molecule, (i.e. a molecule that inhibits immune response), and the immune checkpoint modulator is an inhibitor (an antagonist) of an inhibitory immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In certain embodiments, the immune checkpoint modulator is capable of binding to, or modulating the activity of more than one immune checkpoint. Examples of stimulatory and inhibitory immune checkpoints, and molecules that modulate these immune checkpoints that may be used in the methods of the invention, are provided below.

In certain embodiments, the immune checkpoint modulator stimulates the immune response of the subject. For example, in some embodiments, the immune checkpoint modulator stimulates or increases the expression or activity of a stimulatory immune checkpoint (e.g. CD28, CD122, ICOS, OX40, CD2, CD27, ICAM-1, NKG2C, SLAMF7, NKp80, B7-H3, LFA-1, 1COS, 4-1BB, GITR, CD30, CD40, BAFFR, HVEM, CD7, LIGHT, or CD83). In some embodiments, the immune checkpoint modulator inhibits or decreases the expression or activity of an inhibitory immune checkpoint (e.g. A2A4, B7-H3, B7-H4, IDO, KIR, PD-1, PD-L1, PD-L2, CTLA-4, TIM-3, LAG3, CD160, 2B4, TGF β, VISTA, BTLA, TIGIT or LAIR1).

In a particular embodiment, the immune checkpoint modulator targets an immune checkpoint molecule of PD-1. In a particular embodiment, the immune checkpoint modulator targets an immune checkpoint molecule of PD-L1. In a particular embodiment, the immune checkpoint modulator targets an immune checkpoint molecule of CTLA-4.

In some embodiments, more than one (e.g. 2, 3, 4, 5 or more) immune checkpoint modulator is administered to the subject. Where more than one immune checkpoint modulator is administered, the modulators may each target a stimulatory immune checkpoint molecule, or each target an inhibitory immune checkpoint molecule. In other embodiments, the immune checkpoint modulators include at least one modulator targeting a stimulatory immune checkpoint and at least one immune checkpoint modulator targeting an inhibitory immune checkpoint molecule.

In certain embodiments, the immune checkpoint modulator is a binding protein, for example, an antibody. The term “binding protein”, as used herein, refers to a protein or polypeptide that can specifically bind to a target molecule, e.g. an immune checkpoint molecule. In some embodiments the binding protein is an antibody or antigen binding portion thereof, and the target molecule is an immune checkpoint molecule. In some embodiments the binding protein is a protein or polypeptide that specifically binds to a target molecule (e.g., an immune checkpoint molecule). In some embodiments the binding protein is a ligand. In some embodiments, the binding protein is a fusion protein. In some embodiments, the binding protein is a receptor. Examples of binding proteins that may be used in the methods of the invention include, but are not limited to, a humanized antibody, an antibody Fab fragment, a divalent antibody, an antibody drug conjugate, a scFv, a fusion protein, a bivalent antibody, and a tetravalent antibody, and preferably, the modulator of an immune checkpoint molecule is a monoclonal antibody or an antigen-binding fragment thereof.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In some embodiments, the antibody is a monoclonal antibody. In other embodiments, the antibody is a chimeric antibody. Chimeric and humanized antibodies may be prepared by methods well known to those of skill in the art including CDR grafting approaches (See, e.g., U.S. Pat. Nos. 5,843,708; 6,180,370; 5,693,762; 5,585,089; and 5,530,101), chain shuffling strategies (See, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

The term “antigen-binding portion” or “antigen-binding fragment” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” or “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” or “antigen-binding fragment” of an antibody. Other forms of single chain antibodies, such as diabodies are also encompassed. Antigen binding portions can also be incorporated into single domain antibodies, maxibodies, minibodies, nanobodies, intrabodies, diabodies, triabodies, tetrabodies, v-NAR and bis-scFv (See, e.g., Hollinger and Hudson, Nature Biotechnology 23:1126-1136, 2005).

In some embodiment, the modulator of an immune checkpoint molecule is a monoclonal antibody or an antigen binding fragment thereof. As used herein, a “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (e.g., Fab, Fab′, F(ab′)₂, Fᵥ), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “humanized antibody”, as used herein refers to non-human (e.g., murine) antibodies that are chimeric immunoglobulins, immunoglobulin chains, or fragments thereof (such as Fv, Fab, Fab′, F(ab′)₂ or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, See Jones et al. (1986) NATURE 321: 522-525; Reichmann et al. (1988) NATURE 332: 323-329; and Presta (1992) CURR. OP. STRUCT. BIOL. 2: 593-596, each of which is incorporated by reference herein in its entirety.

The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate.

As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

A “bivalent antibody” refers to an antibody or antigen-binding fragment thereof that comprises two antigen-binding sites. The two antigen binding sites may bind to the same antigen, or they may each bind to a different antigen, in which case the antibody or antigen-binding fragment is characterized as “bispecific.” A “tetravalent antibody” refers to an antibody or antigen-binding fragment thereof that comprises four antigen-binding sites. In certain embodiments, the tetravalent antibody is bispecific. In certain embodiments, the tetravalent antibody is multispecific, i.e. binding to more than two different antigens.

Fab (fragment antigen binding) antibody fragments are immunoreactive polypeptides comprising monovalent antigen-binding domains of an antibody composed of a polypeptide consisting of a heavy chain variable region (V_{H}) and heavy chain constant region 1 (C_{H1}) portion and a poly peptide consisting of a light chain variable (V_{L}) and light chain constant (C_{L}) portion, in which the C_{L} and CH₁ portions are bound together, preferably by a disulfide bond between Cys residues.

In certain embodiments, the immune checkpoint modulator is pembrolizumab, ipilimumab, nivolumab, atezolizumab, avelumab or durvalumab. In certain embodiments, the immune checkpoint modulator is AGEN-1884, BMS-986016, CS-1002, LAG525, MBG453, MEDI-570, OREG-103/BY40, lirilumab, or tremelimumab. In certain embodiments, the immune checkpoint modulator is pembrolizumab, nivolumab, AMP-224, AMP-514, BGB-A317, cemiplimab, JS001, CS1001, PDR-001, PF-06801591, IBI-308, pidilizumab, SHR-1210, or TSR-042. In certain embodiments, the immune checkpoint modulator is atezolizumab, avelumab, durvalumab, AMP-224, JS003, LY3300054, MDX-1105, SHR-1316, KN035, or CK-301.

Modulators of immune checkpoint molecules that can be used in combination with the MDM2 inhibitors as provided herein are described in PCT application WO 2020/030016 A1, which is incorporated herein in its entirety by reference.

In certain embodiments, the method comprises administering the MDM2 inhibitor having the following formula (Compound C) or a pharmaceutically acceptable salt thereof

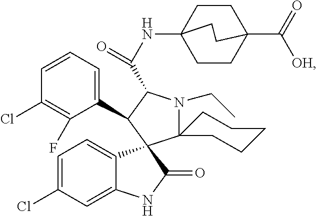 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

and pembrolizumab.

### b) Cancers

In various embodiments of the methods for treating a subject having cancer using MDM2 inhibitor and the modulator of an immune checkpoint molecule, the cancer is selected from the group consisting of melanoma, non-small cell cancer (NSCLC), lung adenocarcinoma, solid tumor with wild-type p53 and ATM mutation, urothalial carcinoma or malignant peripheral nerve sheath tumor (MPNST).

In certain embodiments, the cancer is locally advanced, unresectable or metastatic. As used herein and with respect to cancer, the term “unresectable” refers to a cancer that cannot be removed completely through surgery; the term “locally advanced” refers to a cancer that has grown outside its initial or primary site in but has not yet spread to distant parts of the body; the term “metastatic” refers to a cancer that has spread from its initial or primary site to distant site within the subject.

In some embodiments, the subject having cancer is refractory or relapse of immunotherapy. As used herein and with respect to immunotherapy, the term “refractory” refers to a cancer that is not responding to the treatment and may also be referred to as “resistant”, including cancer that is resistant at the beginning of the treatment and cancer that become resistant during the treatment; the term “relapse” refers to the return of cancer despite a period of improvement made in the treatment.

An immunotherapy can comprise a modulator of immune checkpoint molecule. Examples of immune checkpoint molecule include, without limitation, PD-1, PD-L1, PD-L2, CTLA-4, TIM-3, LAG3, CD160, 2B4, TGFβ, VISTA, BTLA, TIGIT, OX40, CD2, CD27, CDS, ICAM-1, NKG2C, SLAMF7, NKp80, B7-H3, LFA-1, 1COS, 4-1BB, GITR, CD30, CD40, BAFFR, HVEM, CD7, LIGHT or CD83 ligand, and preferably, the immune checkpoint molecule is PD-1, PD-L1 or CTLA-4. In some embodiments, the immunotherapy is anti-PD-1 therapy or anti-PD-L1 therapy. Anti-PD-1/PD-L1 therapy can include, for example, treatment with anti-PD-1 antibody, or anti-PD-L1 antibody.

In some embodiments, the subject is selected from the following groups:

(a) patients having unresectable or metastatic melanomas and being refractory or relapse after immunotherapy (such as the treatment with anti-PD-1 or anti-PD-L1 antibody);
(b) patients having unresectable or metastatic NSCLC and being refractory or relapse after the treatment with immunotherapy (such as the treatment with anti-PD-1 or anti-PD-L1 antibody);
(c) patients having unresectable or metastatic lung adenocarcinoma with STK-11 mutation, and optionally being refractory or relapse after the treatment with immunotherapy (such as the treatment with anti-PD-1 or anti-PD-L1 antibody);
(d) patients having a solid tumor with functional (such as wild-type) p53 and ATM mutation (such as germline ATM mutation or somatic ATM mutation);
(e) patients having has locally advanced or metastatic liposarcomas with functional (such as wild-type) p53 and MDM2 amplification;
(f) patients having unresectable or metastatic urothelial carcinoma without FGFR translocation and/or point mutation and being refractory or relapse after the treatment with immunotherapy (such as the treatment with anti-PD-1 or anti-PD-L1 antibody); and
(g) patients having unresectable or metastatic MPNST.

“STK-11” as used herein refers to serine/threonine-protein kinase STK-11, which is also known as, e.g., polarization-related protein LKB1. STK-11 plays roles in various process such as cell metabolism, cell polarity, apoptosis and DNA damage response by activating targets including 5′-adenosine monophosphate-activated protein kinase (AMPK) and the AMPK-related kinases by direct phosphorylation (Williams T., et al., Trends in cell Biology, (2008)18:193). Exemplary sequence of human STK11 is available in UniProtKB database under the accession number of Q15831 (STK11-HUMAN), in the GenBank database under the NCBI accession number of AAB97833.1.

In some embodiments, STK-11 mutation comprises one or more inactivating mutations. Inactivating mutation of STK-11 can lead to partial or complete loss of the activity and/or expression level of STK-11 by affecting, e.g., coding sequence, RNA splicing sites, promoter or other cis-regulatory elements of STK-11 gene. In certain embodiments, the inactivating mutation reduces activity of STK-11, for example, the serine/threonine kinase activity of STK-11. In certain embodiments, the inactivating mutation reduces STK-11 mRNA expression or protein expression level. The inactivating mutation in STK-11 can be a deletion, insertion, substitution or any combination thereof. Numerous mutations in STK-11 have been identified across various cancer samples, and are available in databases such as Catalogue of Somatic Mutations in Cancer (COSMIC). STK-11 mutations associated with lung adenocarcinoma include, but not limited to S19W, D30N, Y49D, K78A, K78E, K78I, K78N, R86G, H107L, I111N, Q123R, C132F, M136R, F157S, L160P, G163D, H168R, Q170P, G171S, H174D, H174R, H174Y, K175 D176del, D176A, D176H, D176N, D176Y, I177N, I177T, P179Q, P179R, P179S, N181E, N1811, N181Y, L182P, L183V, S193F, D194E, D194H, D194N, D194Y, L195M, G196V, E223V, T230P, S232P, V236A, D237N, D237Y, or W239C.

The term “functional p53”, as used herein, refers to wild-type p53 and mutant or allelic variants of p53 that retain at least about 5% of the activity of wild-type p53, e.g., at least about 10%, about 20%, about 30%, about 40%, about 50%, about 60%, about 70%, or more of wild-type p53 activity. p53 used as the biomarker herein can be p53 protein as well as a polynucleotide (e.g. DNA or RNA) encoding the p53 protein.

“ATM” as used herein is short for ataxia telangiectasia mutated, or ATM serine/threonine kinase. ATM has been suggested as a major post translational regulator of MDM2, and mutation in ATM may lead to similar effects induced by MDM2 overexpression (see, for example, Cheng Q et al, EMBO J, 28: 3857-3867 (2009); and Maya R et al, Gene Dev. 15: 1067-1077 (2001)). Exemplary sequence of human ATM is available in UniProtKB database under the accession number of Q13315 (ATM-HUMAN), in the GenBank database under the NCBI accession number of AAB65827.

In some embodiments, ATM mutation comprises one or more inactivating mutations. Inactivating mutation of ATM can lead to partial or complete loss of the activity and/or expression level of ATM by affecting, e.g., coding sequence, RNA splicing sites, promoter or other cis-regulatory elements of ATM gene. In certain embodiments, the inactivating mutation reduces activity of ATM, for example, the serine/threonine kinase activity of ATM. In certain embodiments, the inactivating mutation reduces ATM mRNA expression or protein expression level. The inactivating mutation in ATM can be a deletion, insertion, substitution or any combination thereof.

In some embodiments, the ATM inactivating mutation is a germline mutation that is inherited and presents in both somatic and germline cells in the offspring. In some embodiments, the ATM inactivation mutation is a somatic mutation that is not inherited and does not affect the germline. Both germline ATM mutations and somatic ATM mutations have been observed in a broad range of solid tumors and hematologic malignancies, see, for example, Choi M, et al., Molecular Cancer Therapeutics, (2016)15:1781). Somatic ATM mutations are available in databases such as Catalogue of Somatic Mutations in Cancer (COSMIC). In some embodiments, ATM mutations associated with solid tumor comprises R43Q, R45Q, S49C, W57*, A59S, V60F, P80S, S99G, L100W, I124V, D126E, S131*, F168L, P178S, A220V, R2211, K224E, R248Q, R250*, L259I, V278fs, P292L, K293*, A302fs, S333F, R337C, R337H, R337S, L348_M349insYIV, D351Y, C353fs, E390*, V410A, W412*, C430S, K468Efs*18, K468fs, G514D, V519I, E522*, C532Y, C540*, L546V, F570L, L581V, F582L, P604S, V630M, P631S, S707P, S719*, C730Y, N765Kfs*12, L804fs, R805*, L822V, S824F, R832C, E848Q, F858L, E871K, F897I, N914S, L9421, P960H, S978A, S978C, S978fs, S978P, L991S, F1025L, L1046R, P1054R, E1072*, Y1124F. The abbreviations of the mutations are known in the art. For instance, “R43Q” denotes that the amino acid residue 43 Arginine (R) is changed to a Glutamine (Q); “W57*” denotes that the nucleotides encoding amino acid residue 57 Tryptophan (W) is changed to a stop codon and the resultant polypeptide is prematurely truncated; “V278fs” denotes a frame shifting with the amino acid residue 278 Valine (V) as the first affected amino acid residue; “K468Efs*18” denotes a frame shifting starting at the amino acid residue 468 Lysine (K) as the first affected amino acid residue and terminating 18 residues downstream. L348_M349insYIV” denotes an insertion of the amino acid sequence YIV between the amino acids residues 348 Leucine (L) and 349 Methionine (M).

“MDM2 amplification” as used herein refers to increase in the copy number of MDM2 gene without a proportional increase in other genes. MDM2 amplification has been detected in many human malignancies, such as lung cancer, colon cancer and other malignancies, and is associated with chemotherapeutic resistance in human malignancies (see Hou, et al., Cancer Cell Int, (2019) 19: 216).

“FGFR” as used herein refers to fibroblast growth factor receptors, a group of receptors that bind to members of the fibroblast growth factor (FGF) family of proteins. Several distinct FGFRs (such as FGFR1 to FGFR4) have been identified in humans and all belong to the tyrosine kinase superfamily. The normal regulation of FGFRs signaling may be subverted to constitutively activate the pathway in a variety of malignances. FGFRs activation can be attributed to genetic alterations such as gene amplification, chromosomal translocation, alternative splicing or point mutation (Karkera J D., et al., Molecular Cancer Therapeutics. (2017) 16:1717). FGFR genetic alteration is observed in urothelial carcinoma (Carneiro B A, et al., Cancer Treatment Reviews, (2014)41: 170, Necchi A., et al, Eur Urol Focus, (2017) http://dx.doi.org/10.1016/j.euf.2017.08.002). The term “translocation” as used herein refers to a genetic mutation in which a piece of one chromosome breaks off and attaches to another chromosome. The term “point mutation” as used herein refers to genetic mutation where a single nucleotide base is changed, inserted or deleted. In some embodiments, the unresectable or metastatic urothelial carcinoma is without FGFR translocation and/or point mutation.

Mutations (including translocation mutation, amplification) in the above referenced genes can be indicated by any methods known in the art, including but not limited to, a hybridization assay (e.g., Northern blotting, Southern blotting, in-situ hybridisation, microarray analysis), a sequencing assay (e.g., RNA sequencing, whole exome sequencing, pyrosequenceing, Sanger sequencing, high throughput sequencing), an amplification assay (e.g., polymerase chain reaction (PCR), reverse transcription PCR, quantitative PCR, ligase chain reaction, isothermal amplification), an immumoassay (e.g., Western blotting, enzyme-linked immunosorbent assay (ELISA), radioimmunoassay, immunofluorescent staining and imaging) or a protein activity assay. For examples, the nucleic acid amplification assay allows the detection of the DNA copy number variation (e.g. gene amplification) or the alteration in mRNA expression level; the nucleic acid hybridization assay can be used to detect the presence of specific DNA or RNA sequences (e.g. DNA or RNA sequences bearing specific mutations); the nucleic acid sequencing assay can be used to determine the sequence of the target nucleic acid (e.g. DNA or RNA), and, if in the format of pyrosequencing or high through put sequencing, to measure the level of the target nucleic acid by enumeration of the sequenced nucleic; the immunoassay can be used to detect or measure the presence or level of the target polypeptide using antibodies that specifically bind to the target polypeptide.

Methods of using STK-11, ATM and/or MDM2 as a biomarker for selecting subject having cancer for the treatment with a MDM2 inhibitor is disclosed in PCT applications PCT/CN2020/077442, PCT/CN2020/076094, which are incorporated herein in their entirety by reference.

MPNST are aggressive soft tissue sarcomas associated with a high risk of recurrence and metastasis. MPNST account for 10% of all soft tissue sarcomas and carry the highest risk for sarcoma specific death among all the soft tissue histologies (Kattan M W, et al., J. Clin. Oncol., (2002)20: 791). At present, complete surgical resection is the only known curative treatment for MPNST and conventional chemotherapy and radiation have not shown to reduce mortality in inoperable tumors (Ferrari A, et al., Eur. J. Cancer, (2011)47: 724; Scaife C L, et al., Surg. Oncol. Clin. N. Am. (2003)12:243). The outcome for unresectable, metastatic or recunent MPNST remain dismal.

Most malignant peripheral nerve sheath tumors (MPNSTs) carry genetic alterations at the CDKN2A locus, which encodes two tumor suppressor genes, pl6INK4A (an inhibitor for D-type cyclins/CDK4 6) and pl4ARF. Importantly, pl4ARF is a negative regulator of MDM2 by multiple mechanisms, whose loss increases MDM2 activity and thus decreases p53 functions. Consistently, most MPNSTs carry wild-type TP53, making it a candidate for MDM2 inhibitor trial. Furthermore, immunoprofiles of MPNSTs revealed high levels of M2-type macrophages with high levels of PD-L1 expression.

### IV. Administration Regimen

### a) Administration of MDM2 Inhibitors

The disclosure described herein apply to MDM2 inhibitor (e.g., Compound C) as a single agent treatment or as a co-agent in the combination therapy. In the methods of the invention, the MDM2 inhibitor (e.g., Compound C) can be administered in the form of a pharmaceutical composition that comprises the MDM2 inhibitor as the single therapeutic ingredient, or a pharmaceutical composition that further comprises at least one additional therapeutic agent.

One skilled in the art would be able, by routine experimentation, to determine what an effective, non-toxic amount of a MDM2 inhibitor (e.g., Compound C) would be for the purpose of treating cancers. For example, a therapeutically active amount of MDM2 inhibitor (e.g., Compound C) may vary according to factors such as the disease stage (e.g., stage I versus stage IV), age, sex, medical complications (e.g., immunosuppressed conditions or diseases) and weight of the subject, and the ability of the MDM2 inhibitor (e.g., Compound C) to elicit a desired response in the subject. The dosage regimen may be adjusted to provide the optimum therapeutic response. For example, several divided doses may be administered daily or administered by continuous infusion or the dose may be proportionally reduced as indicated by the exigencies of the therapeutic situation. In certain embodiments, a MDM2 inhibitor (e.g., Compound C) is administered in an amount that would be therapeutically effective if delivered alone, i.e., MDM2 inhibitor (e.g., Compound C) is administered and/or acts as a therapeutic anti-cancer agent, and not predominantly as an agent to ameliorate side effects of other chemotherapy or other cancer treatments.

In certain embodiments, a MDM2 inhibitor (e.g., Compound C) is administered in an amount that would be effective to improve or augment the immune response to the tumor, e.g., by augmenting the therapeutic effect of one or more immune checkpoint modulators. In certain embodiments, a MDM2 inhibitor (e.g., Compound C) is administered in an amount that would be effective to improve or augment the therapeutic effects of a Bcl-2 inhibitor or a Bcl-2/Bcl-xL inhibitor. The dosages provided below may be used for any mode of administration of MDM2 inhibitor (e.g., Compound C), including oral administration, topical administration, administration by inhalation, and intravenous administration (e.g. injection or continuous infusion).

In some embodiments, when used as a co-agent in the combination therapy, the MDM2 inhibitor (e.g., Compound C) is administered at a dosage that is different (e.g. lower) from the standard dosages of the MDM2 inhibitor used to treat the oncological disorder under the standard of care for treatment for a particular oncological disorder. In certain embodiments, the administered dosage of the MDM2 inhibitor (e.g., Compound C) is 5%, 10%, 20%, 30%, 40%, 50%, 60%, 70%, 80%, or 90% lower than the standard dosage of the MDM2 inhibitor (e.g., Compound C) molecule for a particular oncological disorder. In certain embodiments, the dosage administered of the MDM2 inhibitor (e.g., Compound C) molecule is 95%, 90%, 85%, 80%, 75%, 70%, 65%, 60%, 55%, 50%, 45%, 40%, 35%, 30%, 25%, 20%, 15%, 10% or 5% of the standard dosage of the MDM2 inhibitor (e.g., Compound C) molecule for a particular cancer.

In some embodiments, a MDM2 inhibitor, such as Compound C, or pharmaceutically acceptable salt thereof, is administered orally every day (QD) or every other day (QOD). In some embodiments, the MDM2 inhibitor, such as Compound C or pharmaceutically acceptable salt thereof, is administered orally in an amount from about 30 mg to about 250 mg every day or every other day. In some embodiments, the MDM2 inhibitor, such as Compound C, or pharmaceutically acceptable salt thereof, is administered orally in an amount from about 50 mg to about 250 mg every day or every other day. In some embodiments, the MDM2 inhibitor, such as Compound C, or pharmaceutically acceptable salt thereof, is administered orally in an amount of about 50 mg, 100 mg, 150 mg, 200 mg, or 250 mg every day or every other day.

In some embodiments, a MDM2 inhibitor, such as Compound C, or pharmaceutically acceptable salt thereof, is administered, in at least one 21-day or 28-day treatment cycle.

In certain embodiments, the MDM2 inhibitor, such as Compound C, or pharmaceutically acceptable salt thereof, is administered in at least one 28-day treatment cycle, wherein the MDM2 inhibitor is administrated at an effective amount orally every day on certain days (such as the first consecutive 5-days) of the treatment cycle, wherein the effective amount of the MDM2 inhibitor is about 50 mg to about 250 mg (such as about 50 mg, 100 mg, 150 mg, 200 mg, or 250 mg).

In certain embodiments, the MDM2 inhibitor, such as Compound C, or pharmaceutically acceptable salt thereof, is administered in at least one 21-day or 28-day treatment cycle, wherein the MDM2 inhibitor is administrated at an effective amount orally every other day for the first consecutive 2-weeks of the 21-day or 28-day treatment cycle, or for the first consecutive 3-weeks of the 28-day treatment cycle, wherein he effective amount of the MDM2 inhibitor is about 50 mg to about 250 mg (such as about 50 mg, 100 mg, 150 mg, 200 mg, or 250 mg).

In certain embodiments, methods of treating T-cell prolymphocytic leukemia (T-PLL) in a subject in need thereof, comprising administering about 50 mg, 100 mg, 150 mg, 200 mg, or 250 mg Compound C to the subject orally every day on days 1 to 5 of a 28-day treatment cycle.

### b) Co-Administration of a MDM2 Inhibitor and an Additional Therapeutic Agent.

In some embodiment, the MDM2 inhibitor is administered prior to, concurrently or substantially concurrently with, subsequently to, intermittently with, or in any order relative to the administration of an additional therapeutic agent (such as a Bcl-2 inhibitor or a Bcl-2/Bcl-xL inhibitor or a modulator of an immune checkpoint molecule).

A MDM2 inhibitor (e.g., Compound C) and/or pharmaceutical compositions thereof and an additional therapeutic agent (e.g., an immune checkpoint modulator or a Bcl-2 inhibitor or a Bcl-2/Bcl-xL inhibitor) can act additively or, more preferably, synergistically. In one embodiment, the MDM2 inhibitor (e.g., Compound C) and the additional therapeutic agent act synergistically. In some embodiments the synergistic effects are in the treatment of the cancer. For example, in one embodiment, the combination of a MDM2 inhibitor (e.g., Compound C) and the additional therapeutic agent improves the durability, i.e. extends the duration, of the response against the cancer that is targeted by the therapy. In other embodiments the synergistic effects are in modulation of the toxicity associated with the additional therapeutic agent. In one embodiment, a MDM2 inhibitor (e.g., Compound C) and the additional therapeutic agent act additively.

The combination therapies of the present invention may be utilized for the treatment of cancers. In some embodiments, the combination therapy of a MDM2 inhibitor (e.g., Compound C) and an additional therapeutic agent inhibits tumor cell growth. Accordingly, the invention further provides methods of inhibiting tumor cell growth in a subject, comprising administering a MDM2 inhibitor (e.g., Compound C) and at least one additional therapeutic agent to the subject, such that tumor cell growth is inhibited. In certain embodiments, treating cancer comprises extending survival or extending time to tumor progression as compared to control, e.g., a population control. In certain embodiments, the subject is a human subject. In preferred embodiments, the subject is identified as having a tumor prior to administration of the first dose of a MDM2 inhibitor (e.g., Compound C) or the first dose of the additional therapeutic agent In certain embodiments, the subject has a tumor at the time of the first administration of a MDM2 inhibitor (e.g., Compound C) or at the time of first administration of the additional therapeutic agent.

### (1) Co-Administration of a MDM2 Inhibitor and a Bcl-2 Inhibitor or a Bcl-2/Bcl-xL Inhibitor.

In certain embodiments, the present disclosure provides methods of treating T-PLL comprises administrating a therapeutically effective amount of MDM2 inhibitor and a therapeutically effective amount of the Bcl-2 inhibitor (such as Compound C6) or the Bcl-2/Bcl-xL inhibitor (such as Compound A15 or B4).

In some embodiments, a therapeutically effective amount of a Bcl-2 inhibitor (such as Compound C6) or a Bcl-2/Bcl-xL inhibitor (such as Compound A15 or B4) can be administered to the patient before, after or simultaneously with the administration of a therapeutically effective amount of the MDM2 inhibitor (Compound C) to the subject in need thereof. In some embodiments, the Bcl-2 inhibitor (such as Compound C6) or the Bcl-2/Bcl-xL inhibitor (such as Compound Aly or B4) and the MDM2 inhibitor (Compound C) can each be combined with a pharmaceutically acceptable carrier.

In some embodiments, the Bcl-2 inhibitor and the MDM2 inhibitor may be administered sequentially at a time interval of about 1 minute, about 5 minutes, about 10 minutes, about 15 minutes, about 30 minutes, about 45 minutes, about 1 hour, about 2 hours, about 4 hours, about 6 hours, about 12 hours, about 24 hours, about 48 hours, about 72 hours, about 96 hours, about 1 week, about 2 weeks, about 3 weeks, about 4 weeks, about 5 weeks, about 6 weeks, about 8 weeks, or about 12 weeks.

In some embodiments, the Bcl-2 inhibitor (such as Compound C6) or the Bcl-2/Bcl-xL inhibitor (such as Compound A15 or B4) and the MDM2 inhibitor (Compound C) can be administered together as a single unit dose or separately as multiple unit doses.

In some embodiments, the single unit dose or the separate unit dose may be administered for, including, but not limited to, 1 time, 2 times, 3 times, 4 times, 5 times or 6 times.

In certain embodiments, the Bcl-2/Bcl-xL inhibitor (such as Compound A15 or B4) is administered at a dose from about 10 mg/day to about 1000 mg/day, from about 10 mg/day to about 900 mg/day, from about 10 mg/day to about 800 mg/Day, about 10 mg/day to about 700 mg/day, about 10 mg/day to about 640 mg/day, about 10 mg/day to about 600 mg/day, about 10 mg/day to about 500 mg/day, about 10 mg/day to about 400 mg/day, about 10 mg/day to about 300 mg/day, about 10 mg/day to about 200 mg/day, or about 20 mg/day to about 100 mg/day, for example about 10, 15, 20, 25, 30, 35, 40, 50, 55, 60, 65, 70, 75, 80, 85, 90, 95, 100, 150, 200, 250, 300, 350, 400, 450, 500, 550, 600, 650, 700, 750, 800, 850, 900, 950, 1000 mg/day. In certain embodiments, the Bcl-2/Bcl-xL inhibitor (such as Compound A15 or B4) is administered at a dose of about 0.005, 0.05, 0.5, 5, 10, 20, 30, 40, 50, 100, 150, 200, 250, 300, 350, 400, 450 or 500 mg per dose. In certain embodiments, the Bcl-2/Bcl-xL inhibitor (such as Compound A15 or B4) is administered at a frequency of once a day or more times a day.

In some embodiments, the Bcl-2 inhibitor (such as Compound C6), is administered in an amount from about 0.0025 to 1500 mg/day. Preferably, the daily dose of the Bcl-2 inhibitor (such as Compound C6) is 1 mg, 5 mg, 10 mg, 20 mg, 30 mg, 40 mg, 50 mg, 60 mg, 61 mg, 70 mg, 80 mg, 90 mg, 100 mg, 122 mg, 150 mg, 200 mg, 244 mg, 250 mg, 300 mg, 350 mg, 400 mg, 450 mg, 460 mg, 470 mg, 480 mg, 487 mg, 490 mg, 500 mg, 550 mg, 600 mg, 650 mg, 700 mg, 750 mg, 800 mg, 850 mg, 900 mg, 950 mg, 1000 mg, and a range between the respective doses, for example, 1 mg to 1000 mg, 30 mg to 900 mg, 61 mg to 800 mg, 100 mg to 700 mg, 122 mg to 600 mg, 122 mg to 500 mg, 122 mg to 487 mg, 122 mg to 300 mg, 122 mg to 244 mg, 30 mg to 487 mg, 61 mg to 487 mg and the like.

In some embodiments, the Bcl-2 inhibitor (such as Compound C6) is administered daily at a dose of 0.017 mg/kg, 0.083 mg/kg, 0.17 mg/kg, 0.33 mg/kg, 0.5 mg/kg, 0.67 mg/kg, 0.83 mg/kg, 1 mg/kg, 1.02 mg/kg, 1.16 mg/kg, 1.33 mg/kg, 1.5 mg/kg, 1.67 mg/kg, 2.03 mg/kg, 2.5 mg/kg, 3.33 mg/kg, 4.06 mg/kg, 4.17 mg/kg, 5 mg/kg, 5.83 mg/kg, 6.67 mg/kg, 7.5 mg/kg, 7.67 mg/kg, 7.83 mg/kg, 8 mg/kg, 8.12 mg/kg, 8.16 mg/kg, 8.33 mg/kg, 9.17 mg/kg, 10 mg/kg, 10.83 mg/kg, 11.66 mg/kg, 12.5 mg/kg, 13.33 mg/kg, 14.17 mg/kg, 15 mg/kg, 15.83 mg/kg, 16.67 mg/kg, and a range between the respective doses, for example, 0.017 mg to 16.67 mg/kg, 0.33 mg to 16.67 mg/kg, 1.02 mg to 15 mg/kg, 1.02 mg to 15 mg/kg, 1.02 to 12.5 mg, 1.02 mg to 10 mg/kg, 1.02 mg to 8.12 mg/kg, 1.02 mg to 4.06 mg/kg, 1.02 mg to 2.03 mg/kg, 2.03 mg to 4.06 mg/kg, etc., and the daily dose of the MDM2 inhibitor is 0.5 mg/kg, 0.67 mg/kg, 0.83 mg/kg, 1 mg/kg, 1.17 mg/kg, 1.22 mg/kg, 2.03 mg/kg, 2.5 mg/kg, 3.33 mg/kg, 4.06 mg/kg, 4.17 mg/kg, 5 mg/kg, 5.83 mg/kg, 6.67 mg/kg, 7.5 mg/kg, 7.67 mg/kg, 7.83 mg/kg, 8 mg/kg, 8.12 mg/kg, 8.16 mg/kg, 8.33 mg/kg, 9.17 mg/kg, 10 mg/kg, and a range between the respective doses, for example, 0.5 mg to 10 mg/kg, 1 mg to 10 mg/kg, 1 mg to 5 mg/kg, 2.5 mg to 8.12 mg/kg, 4.06 mg to 10 mg/kg, 4.06 mg to 8.12 mg/kg, and the like.

In some embodiments, the Bcl-2 inhibitor (such as Compound C6) is administered at an effective amount from about 400 to about 1000 mg every day, or from about 400 mg to about 800 mg every day, for example in an effective amount of about 400 mg, about 600 mg, about 800 mg or about 1000 mg every day. In some embodiments, the Bcl-2 inhibitor (such as Compound C6) is administered at an effective amount at about 400 mg, about 600 mg, or about 800 mg every day.

In some embodiments, the treatment of T-PLL comprises co-administering the Bcl-2 inhibitor (such as Compound C6) or the Bcl-2/Bcl-xL inhibitor (such as Compound A15 or B4), and the MDM2 inhibitor (such as Compound C) in at least one 28-day treatment cycle.

In certain embodiments, the 28-day treatment cycle comprises administering the MDM2 inhibitor (such as Compound C) at an effective amount of about 50 mg, 100 mg, 150 mg, 200 mg, or 250 mg every day on days 1 to 5 of the 28-day treatment cycle, and administering the Bcl-2 inhibitor (such as Compound C6) at an effective amount of about 400 mg, 600 mg, 800 mg every day of the 28-day treatment cycle.

In some embodiments, the treatment of T-PLL further comprises administering the Bcl-2 inhibitor (such as Compound C6) according to a daily step-wise dosing regimen before the initiation of the 28-day treatment cycle.

The term “step-wise dosing regimen” used herein refers to a regimen where a dose different from the standard dose is initially established and then adjusted (e.g, increased or decreased) gradually to establish the standard dose. In some embodiments, the step-wise dosing regimen comprises a daily ramp up schedule that increases a dose to be administered from an initial dose to a final dose or increasing dose with gradual escalation over a predetermined time period. The initial dose may be lower than the standard dose (e.g. a dose that is administered during the 28-day treatment cycle). The final dose may be the same or lower than the standard dose (e.g. a dose that is administered during the 28-day treatment cycle).

In some embodiments, the daily step-wise dosing regimen comprises administering the Bcl-2 inhibitor (e.g. Compound C6) in a first dose of 20 mg to 100 mg for 1 day, and in a second dose of 50 mg to 200 mg for 1 day after the first dose. In some embodiments, the daily step-wise dosing regimen further comprises administering the Bcl-2 inhibitor (e.g. Compound C6) in a third dose of 100 to 400 mg for 1 day after the second dose. In some embodiments, the daily step-wise dosing regimen further comprises administering the Bcl-inhibitor (e.g. Compound C6) in a fourth dose of 200 mg to 800 mg for 1 to 7 days after the third dose.

In some embodiments, the daily step-wise dosing regimen comprises administering the Bcl-2 inhibitor (e.g. Compound C6) in a first dose of 20 mg for 1 day, in a second dose of 50 mg for 1 day after the first dose, and in a third dose of 100 mg for 1 day after the second dose. In some embodiments, the daily step-wise dosing regimen comprises administering the Bcl-2 inhibitor (e.g. Compound C6) in a fourth dose of 200 mg for 1 to 5 days after the third dose. In some embodiments, the fourth dose is administered for 1 day. In some embodiments, the daily step-wise dosing regimen further comprises administering the Bcl-2 inhibitor (e.g. Compound C6) in a fifth dose of 400 mg for 1 day after the fourth dose. In some embodiments, the daily step-wise dosing regimen further comprises administering the Bcl-2 inhibitor (e.g. Compound C6) in a sixth dose of 600 mg for 1 day after the fifth dose.

Without bound to any theory, the daily step-wise dosing regimen helps to reduce the risk of tumor lysis syndrome (TLS) during the treatment. TLS is a common yet dangerous side-effect that can be experienced by patients during administration of a Bcl-2 inhibitor. The daily step-wise dosing regimen described herein comprise a therapeutically effective daily ramp-up dosing regimen that allows to achieve the standard therapeutic dose of the Bcl-2 inhibitor in a short time period while reducing the risk of TLS. Prior to the first dose of Bcl-2 inhibitor (such as Compound C6), subjects may be assessed for TLS risk and may receive appropriate prophylaxis for TLS, including such as hydration and anti-hyperuricemia.

In some embodiments, the method of treating T-cell prolymphocytic leukemia (T-PLL) in a subject in need thereof, comprising administering about 50 mg, 100 mg, 150 mg, 200 mg, or 250 mg Compound C orally every day on days 1 to 5 of a 28-day treatment cycle, and administering about 400 mg, 600 mg, or 800 mg Compound C6 every day of the 28-day treatment cycle, wherein the method further comprises administering Compound C6 according to a daily step-wise dosing regimen before the initiation of the 28-day treatment, wherein the daily step-wise dosing regimen comprises administering Compound C6 in a first dose of 20 mg for 1 day, in a second dose of 50 mg for 1 day after the first dose, in a third dose of 100 mg for 1 day after the second dose, and in a fourth dose of 200 mg for 1 day after the third dose. In some embodiments, the daily step-wise dosing regimen further comprises administering the Compound C6 in a fifth dose of 400 mg for 1 day after the fourth dose. In some embodiments, the daily step-wise dosing regimen further comprises administering the Compound C6 in a sixth dose of 600 mg for 1 day after the fifth dose.

Co-administration of the MDM2 inhibitor and the Bcl-2 inhibitor is described in WO2020024820A1, which is incorporated herein in its entirety by reference.

### (2) Co-Administration of a MDM2 Inhibitor and a Modulator of Immune Checkpoint Molecules

Methods are provided for the treatment of cancers by administering a MDM2 inhibitor in combination with at least one immune checkpoint modulator to a subject in need thereof.

The immune checkpoint modulator can be administered via a variety of routes/mode well known in the art. As will be appreciated by the skilled artisan, the route and/or mode of administration will vary depending upon the desired results. In some embodiments, the immune checkpoint modulator is administered via intravenous infusion.

The doses of the immune checkpoint modulator can be calculated per body weight, e.g., mg/kg body weight, or In some embodiments, the immune checkpoint modulator is administered at a dose from about 0.01 mg/kg to about 20 mg/kg, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0. 3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg. In some embodiments, the immune checkpoint modulator is administered at a dose from about 1 mg to about 2000 mg (equivalent to about 0.01 mg/kg to about 20 mg/kg body weight), e.g., about 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg or 2000 mg.

The amount of the immune checkpoint modulator to be administered can be constant for each dose, or vary with each dose. For example, the immune checkpoint modulator can be administered at an initial dose followed by a follow-on dose that is lower, higher or the same as the initial dose.

In some embodiments, the immune checkpoint modulator can be administered at a frequency that is once, twice, 3 times, 4 times, 5 times, 6 times or 7 times every 1, 2, 3, 4, 5, 6, 7, or 8 weeks.

In certain embodiments, the administration of immune checkpoint modulator comprises at least one treatment cycle (e.g., a treatment cycle consisting of 1, 2, 3, 4, 5, 6, 7, or 8 weeks). The treatment cycle can be repeated up to 12 cycles (e.g., at least 1, 2, 3, 4, 5, 6, 7, 8, 9, 10, 11, or 12 cycles), or as long as a clinical benefit is observed or until there is a complete response, confirmed progressive disease or unmanageable toxicity.

Immune checkpoint modulators may be administered at appropriate dosages to treat the oncological disorder, for example, by using standard dosages. One skilled in the art would be able, by routine experimentation, to determine what an effective, non-toxic amount of an immune checkpoint modulator would be for the purpose of treating cancers. Standard dosages of immune checkpoint modulators are known to a person skilled in the art and may be obtained, for example, from the product insert provided by the manufacturer of the immune checkpoint modulator. Examples of standard dosages of immune checkpoint modulators are provided in Table 2 below. In other embodiments, the immune checkpoint modulator is administered at a dosage that is different (e.g. lower) than the standard dosages of the immune checkpoint modulator used to treat the oncological disorder under the standard of care for treatment for a particular oncological disorder.

TABLE 2
Exemplary Standard Dosages of Immune Checkpoint Modulators

| Immune | Immune | |
|---|---|---|
| Checkpoint | Checkpoint | |
| Modulator | Molecule | Exemplary Standard Dosage |
| Ipilimumab | CTLA-4 | 3 mg/kg administered intravenously |
| (Yervoy ®) | | over 90 minutes every 3 weeks for a |
| | | total of 4 doses. |
| Pembrolizumab | PD-1 | 2 mg/kg administered as an intravenous |
| (Keytruda ®) | | infusion over 30 minutes every 3 weeks |
| | | until disease progression or |
| | | unacceptable toxicity. |
| Nivolumab | PD-1 | 3 mg/kg as an intravenous infusion |
| (OPDIVO ®) | | over 60 minutes every 2 weeks. |
| Atezolizumab | PD-L1 | 1200 mg administered as an intravenous |
| (Tecentriq ®) | | infusion over 60 minutes every 3 weeks |
| Avelumab | PD-L1 | 10 mg/kg as an intravenous infusion |
| (BAVENCIO ®) | | over 60 minutes every 2 weeks. |
| Durvalumab | PD-L1 | 10 mg/kg as an intravenous infusion |
| (IMFINZI ®) | | over 60 minutes every 2 weeks. |

In some embodiments, the combination therapy for treating cancer (such as MPNST) comprises administering the modulator of an immune checkpoint molecule (such as an anti-PD-1 antibody, e.g., pembolizumab) via intravenous infusion in an amount of 200 mg on day 1 of the 21-day treatment cycle.

In some embodiments, the combination therapy for treating cancer (such as MPNST) in a subject in need thereof comprises administering the MDM2 inhibitor, such as Compound C or pharmaceutically acceptable salt thereof, orally every other day in an amount from about 50 mg to about 250 mg (e.g. about 50 mg, 100 mg, 150 mg, 200 mg, or 250 mg) for the first two consecutive weeks (e.g. on days 1, 3, 5, 7, 9, 11, and 13) of a 21-day treatment cycle and is not administered during the third week (e.g. days 14-21) of the treatment cycle, and administering the modulator of an immune checkpoint molecule (such as an anti-PD-1 antibody, e.g., pembolizumab) via intravenous infusion in an amount of 200 mg on day 1 of the 21-day treatment cycle.

In some embodiments, the combination therapy comprises at least 1, 2, 3, 4, 5, 6, 7, 8, 9, 10, 11, or 12 of the 21-day treatment cycle. In some embodiments, the combination therapy continues until disease progression or unacceptable toxicity.

In certain embodiments, at least 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

It should be noted that more than one immune checkpoint modulator e.g., 2, 3, 4, 5, or more immune checkpoint modulators, may be administered in combination with a MDM2 inhibitor (e.g., Compound C). In some embodiments, the two or more immune checkpoint modulators target the same immune checkpoint molecule. In some embodiments, the two or more immune checkpoint modulators each target different immune checkpoint molecules.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### EXEMPLIFICATION

### Example 1. A Phase Ha Study Evaluating the Pharmacokinetics, Safety and Efficacy of Compound C as a Single Agent or in Combination with Compound C6 in Subjects with Relapsed/Refractory T-Cell Prolymphocytic Leukemia (R/R T-PLL)

Methodology:

This is a multi-center, open-label, phase IIa study evaluating the safety, efficacy and pharmacokinetics (PK) of Compound C as a single agent or in combination with Compound C6 in patients with T-PLL. The study consists of two parts. A total of about 24˜36 T-PLL patients are enrolled.

Part 1 (Compound C as a Single Agent)

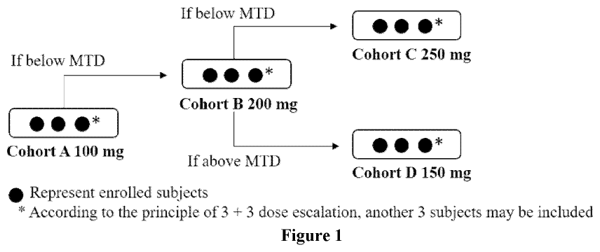

12˜18 patients are enrolled. using a “3+3” dose escalation design. Patients receive Compound C orally once every day (QD) with meal on Days 1 to 5, 23 days off on the 28-day cycles. Compound C dosage starts with 100 mg (Cohort A); if not reach maximum tolerated dose (MTD), the dosage escalates to 200 mg (Cohort B) and 250 mg (Cohort C); if 200 mg is above MTD, an intermediate dose level of 150 mg (Cohort D) will be tested (FIG. 1). MTD is defined as the highest dose with ≤1/6 patients with dose limiting toxicities (DLT). The DLT observation time window is the 1^{st} cycle. Blood samples will be collected from each subject at specified time points to evaluate the PK of Compound C.

Part 2 (Compound C in Combination with Compound C6)

The recommended starting dose of Compound C in combination with Compound C6 in Part 2 will be determined by pooling available dose, PK, PD, safety, and efficacy data of Part 1, and the consequent results of conducting an integrated dose-response and exposure-response analyses on the data from Part 1, after discussion between the sponsor and investigators. Compound C6 will be administered orally QD with meal (low fat meal preferred) in a 28-day treatment cycle following a daily ramp-up schedule (Table 3). All daily ramp-up starts with 20 mg. Dose escalation uses a standard “3+3” design starting from 400 mg to 800 mg (400 mg, 600 mg, 800 mg). Compound C will be given at a fixed dose and started after the daily ramp-up of Compound C6 is completed. MTD is defined as the highest dose with ≤1/6 patients with DLT. The DLT observation time window is also the 1^{st} cycle.

Blood samples will be collected from each patient at specified time points to evaluate the PK of Compound C and Compound C6 when co-administered. Peripheral blood samples will be collected at baseline and post-treatment time points to explore potential predictive and pharmacodynamics (PD) biomarkers

TABLE 3
Part 2 Compound C6 dose ramp-up and combination treatment scheme

| Dose Level | | | | | | | Day 1 and onward |
|---|---|---|---|---|---|---|---|
| Ramp-up (mg) | Day −6 | Day −5 | Day −4 | Day −3 | Day −2 | Day −1 | (28 days per cycle) |
| Compound C6 | — | — | 20 | 50 | 100 | 200 | 400 mg QD Days 1-28 + |
| 400 mg | | | | | | | Compound C QD Days 1-5 |
| Compound C6 | — | 20 | 50 | 100 | 200 | 400 | 600 mg QD Days 1-28 + |
| 600 mg | | | | | | | Compound C QD Days 1-5 |
| Compound C6 | 20 | 50 | 100 | 200 | 400 | 600 | 800 mg QD Days 1-28 + |
| 800 mg | | | | | | | Compound C QD Days 1-5 |

For patients with a certain risk of tumor lysis syndrome (TLS) such as high white blood cells, bulk tumor, and potential renal impairment, investigators can take appropriate prophylaxis based on clinical experience, including but not limited to hydration, anti-hyperuricemia, close laboratory monitoring, and hospitalization if indicated.

Patients enrolled will be monitored for safety and tolerability throughout the study. All patients receiving at least one dose of Compound C alone in Part 1 or Compound C and Compound C6 in Part 2 will be considered evaluable for safety. Treatment-emergent adverse events (TEAS) will be assessed according to National Cancer Institute (NCI) Common Terminology Criteria for Adverse Events (CTCAE) version 5.0 (NCI CTCAE v5.0).

The treatment response of the patients will be assessed at the end of each cycle (28 days) according to 2019 consensus criteria of T-PLL.

### Example 2. A Phase Ib/II Study of Compound C in Combination with Pembrolizumab in Patients with Unresectable or Metastatic Melanomas or Advanced Solid Tumors

Methodology:

This is an open label, multi-center two-part phase Ib/II study. Part 1 is the dose escalation of Compound C in combination with label dose of pembrolizumab. Part 2 is phase II design on Compound C at recommended Phase II dose (RP2D) in combination with pembrolizumab in the patients with PD1/PD-L1 refractory/relapse melanoma or NSCLC, solid tumors with wild-type P53 and ATM mutation, wild-type P53 and MDM2 amplification liposarcomas, PD1/PD-L1 refractory/relapsed urothelial carcinoma without FGFR translocation mutation, or malignant peripheral nerve sheath tumor (MPNST).

Part 1

A phase Ib standard “3+3” design will be employed to determine the MTD of Compound C by assessing the DLT of Compound C in combination with pembrolizumab. Four dose levels of Compound C will be tested: 50, 100, 150, and 200 mg. Compound C will be administered orally every other day (QOD) for consecutive 2 weeks (i.e., dosed at Day 1, 3, 5, 7, 9, 11, and 13), with one week off dosing as 3-weeks a cycle. Pembrolizumab is administered following the FDA approved label dose, i.e.: 200 mg intravenous infusion at Day 1 of every 3 weeks as a cycle. Doses and schedule of Compound C administration may be modified depending on the toxicity and PK results after each dose cohort based on discussions between the investigators and the sponsor.

Dose escalation will be based on DLTs observed in the first 3 weeks (1 cycle) post first dose, with decisions made by both the investigator and the sponsor. If no DLTs are observed by the end of the DLT period in the first 3 patients, the dose of Compound C will be increased in subsequent cohorts accordingly. If ≥2/6 patients develop DLTs at any dose level dose escalation will cease and the dose level immediately below will be expanded to 6 patients. If ≤1/6 patients develop a DLT at the highest dose reached this will be declared the MTD. Patients who are not evaluable for DLTs and/or withdraw during the first cycle for any reason other than a DLT and do not receive more than 70% of the planned dose in DLT observational window will be considered as non-evaluable and be replaced.

Part 2

Part 2 is a phase II study design, includes cohort A-E five arms. The patients will be treated with Compound C at 150 mg QOD (RP2D) in combination with pembrolizumab until disease progression, unacceptable toxicity, or another discontinuation criterion is met.

a. Cohort A: Compound C in combination with pembrolizumab in patients with unresectable or metastatic melanomas, and refractory or relapse after PD1/PDL1 antibody treatment. It will be Simon's two-stage design: 16 for stage 1, 18 for stage 2 (n=34).

b. Cohort B: Compound C in combination with pembrolizumab in patients with unresectable or metastatic NSCLC, and refractory or relapsed after PD1/PDL1 antibody treatment (n=15); Compound C in combination with pembrolizumab in patients with unresectable or metastatic lung adenocarcinoma with STK-11 mutation, and with or without previous PD1/PD-L1 antibody treatment (n=up to 10). Total number of patients in cohort B is up to 25.

c. Cohort C: Compound C in combination with pembrolizumab in patients with unresectable or metastatic solid tumors with wild-type P53 and ATM mutation (including germline ATM mutation) (n=20).

d. Cohort D: Compound C in combination with pembrolizumab in patients with locally advanced or metastatic liposarcomas with wild-type P53 and MDM2 amplification (n=15).

e. Cohort E: Compound C in combination with pembrolizumab in patients with unresectable or metastatic urothelial carcinoma, without FGFR translocation mutation, and refractory or relapse after PD1/PDL1 antibody treatment (n=15).

f. Cohort F: Compound C in combination with pembrolizumab in patients with metastatic or unresectable MPNST (n=10).

What is claimed is:

1. A method of treating T-cell prolymphocytic leukemia (T-PLL) in a subject in need thereof, comprising administering an effective amount of a MDM2 inhibitor, wherein the MDM2 inhibitor is a compound of the following formula (VI), or a pharmaceutically acceptable salt thereof: 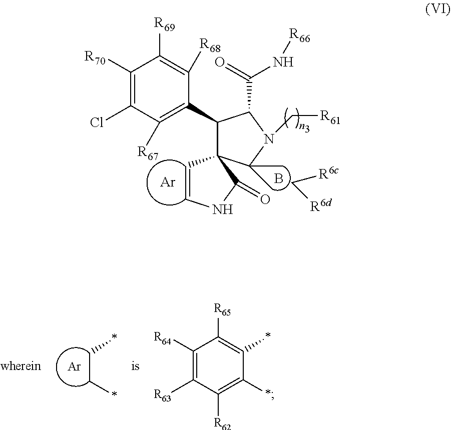
B is 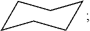 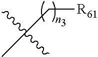
is H, CH₃, or CH₂CH₃;
R₆₂ is H, R₆₃ is F or Cl, and R₆₄ and R₆₅ are H;
R₆₇ is fluoro, each of R₆₈, R₆₉, and R₇₀ is H;
R₆₆ is 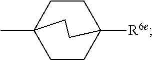
and
R^{6c} is H, CH₃, OH or halo, and R^{6d} is H, CH₃, OH or halo;
R^{6e} is —C(═O)R^{6a}, —C(═O)NR^{6a}R^{6b}, or —C(═O)NHSO₂CH₃;
R^{6a} is hydrogen or unsubstituted C₁₋₄ alkyl; and
R^{6b} is hydrogen or unsubstituted C₁₋₄ alkyl.

2-7. (canceled)

8. The method of claim 1, wherein the MDM2 inhibitor is selected from the following compound or a pharmaceutically acceptable salt thereof:

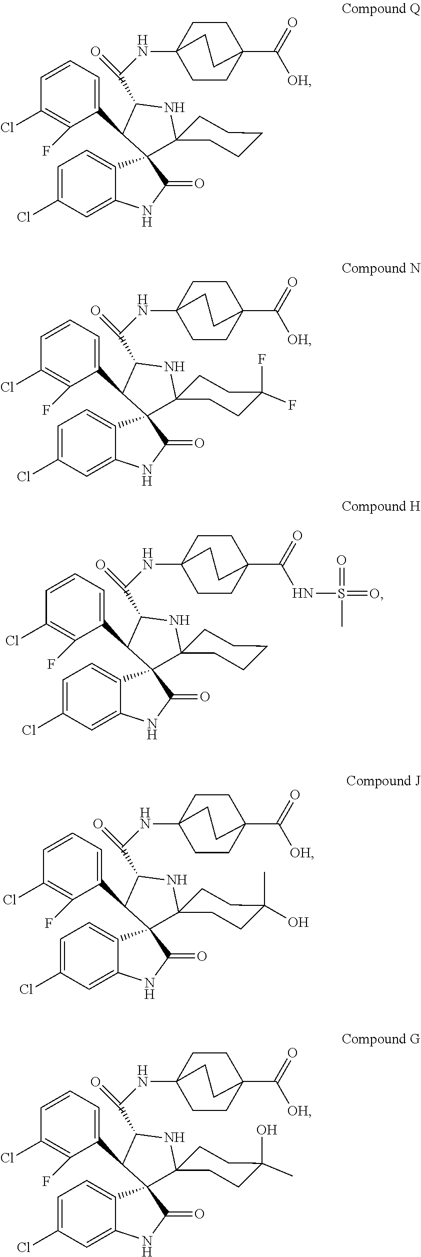 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

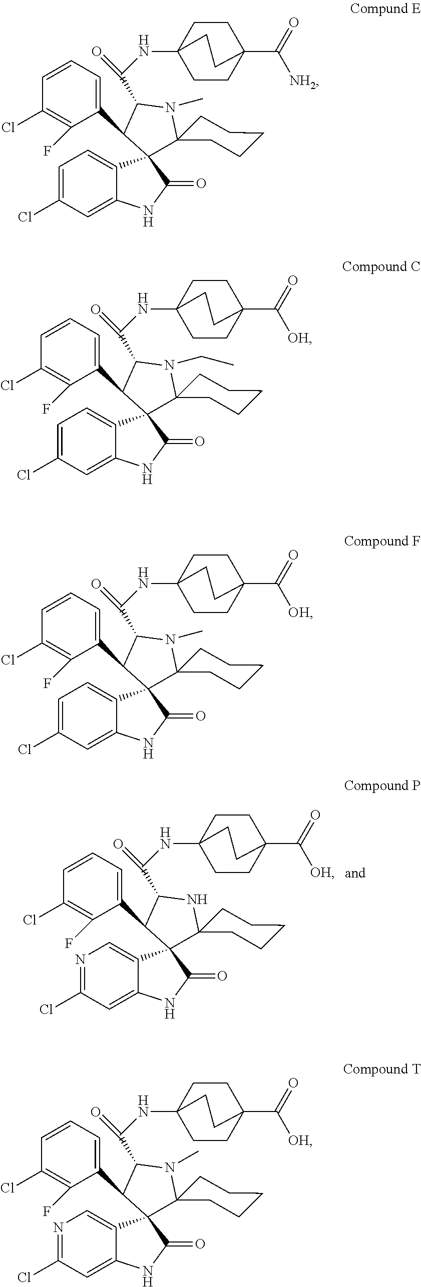 [SMILES: CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2]

9. The method of claim 1, wherein the MDM2 inhibitor is the compound having the following formula or a pharmaceutically acceptable salt thereof:

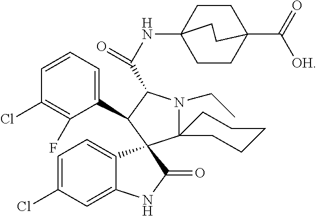 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

10. The method of claim 1, further comprising administering an effective amount of a Bcl-2 inhibitor or a Bcl-2/Bcl-xL inhibitor, wherein the Bcl-2 inhibitor or the Bcl-2/Bcl-xL inhibitor is a compound selected from Table 1A, 1B, 1C, or a pharmaceutically acceptable salt thereof.

11-12. (canceled)

13. The method of claim 10, wherein the Bcl-2 inhibitor is a compound of the following formula, or a pharmaceutically acceptable salt thereof,

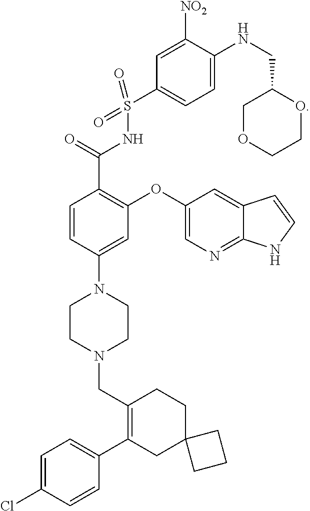 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

14. The method of claim 10, wherein the MDM2 inhibitor is the compound having the following formula or a pharmaceutically acceptable salt thereof: 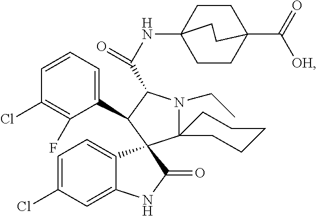 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]
and
the Bcl-2 inhibitor is a compound of the following formula, or a pharmaceutically acceptable salt thereof

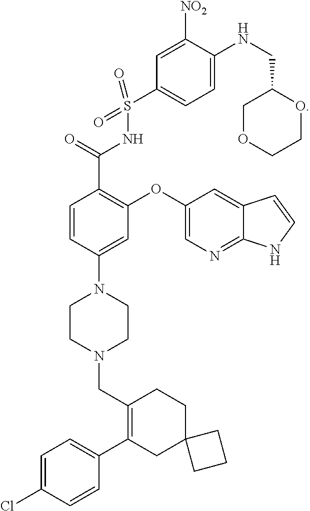 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

15. (canceled)

16. The method of claim 1, wherein the MDM2 inhibitor is administered orally every day, preferably in an effective amount from about 1 mg to about 300 mg every day, more preferably in an amount of about 50 mg, 100 mg, 150 mg, 200 mg or 250 mg every day.

17. The method of claim 1, wherein the treatment comprises at least one 28-day treatment cycle, wherein the MDM2 inhibitor is administered orally every day in a patient in need thereof on days 1 to 5.

18. The method of claim 10, wherein the Bcl-2 inhibitor is administered at an effective amount from about 400 mg to about 1000 mg every day, preferably at about 400 mg, about 600 mg, or about 800 mg every day.

19. The method of claim 10, wherein the Bcl-2 inhibitor is administered orally once every day in the 28-day treatment cycle; optionally the method further comprises administering the Bcl-2 inhibitor according to a daily step-wise dosing regimen before the initiation of the 28-day treatment cycle; preferably, the daily step-wise dosing regimen comprises administering the Bcl-2 inhibitor in a first dose of 20 mg to 100 mg for 1 day, and in a second dose of 50 mg to 200 mg for 1 day after the first dose; optionally the daily step-wise dosing regimen further comprise administering the Bcl-2 inhibitor in a third dose of 100 to 400 mg for 1 day after the second dose, and in a fourth dose of 200 mg to 800 mg for 1 to 7 days after the third dose;
more preferably, the daily step-wise dosing regimen comprises administering the Bcl-2 inhibitor in a first dose of 20 mg for 1 day, in a second dose of 50 mg for 1 day after the first dose, in a third dose of 100 mg for 1 day after the second dose, and in a fourth dose of 200 mg for 1 to 5 days after the third dose; preferably the fourth dose is administered for 1 day.

20-21. (canceled)

22. The method of claim 19, wherein the daily step-wise dosing regimen further comprises administering the Bcl-2 inhibitor in a fifth dose of 400 mg for 1 day after the fourth dose; optionally the daily step-wise dosing regimen further comprises administering the Bcl-2 inhibitor in a sixth dose of 600 mg for 1 day after the fifth dose.

23. A method of treating a subject having cancer, comprising administering an effective amount of a MDM2 inhibitor and a modulator of an immune checkpoint molecule, wherein the subject has melanoma, non-small cell cancer (NSCLC), lung adenocarcinoma, solid tumor with wild-type p53 and ATM mutation, urothalial carcinoma and malignant peripheral nerve sheath tumor (MPNST), wherein the MDM2 inhibitor is a compound of formula (VI), or a pharmaceutically acceptable salt thereof, 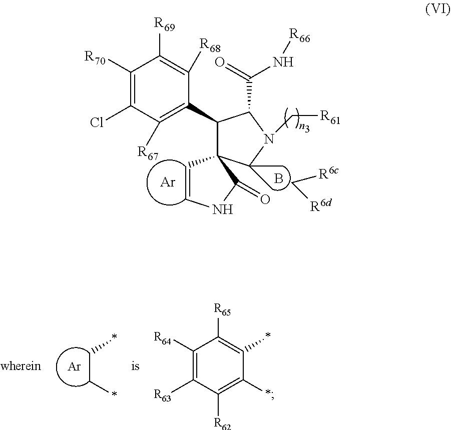
B is 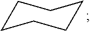 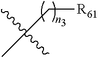
is H, CH₃, or CH₂CH₃;
R₆₂ is H, R₆₃ is F or Cl, and R₆₄ and R₆₅ are;
R₆₆ is 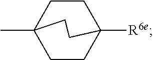
and
R₆₇ is fluoro, each of R₆₈, R₆₉ and R₇₀ is H;
R^{6c} is H, CH₃, OH or halo, and R^{6d} is H, CH₃, OH or halo;
R^{6e} is —C(═O)NR^{6a}, —C(═O)NR^{6a}R^{6b}, or —C(═O)NHSO₂CH₃.
R^{6a} is hydrogen or unsubstituted C₁₋₄ alkyl; and
R^{6b} is hydrogen or unsubstituted C₁₋₄ alkyl.

24-28. (canceled)

29. The method of claim 23, wherein the MDM2 inhibitor is a compound selected from the following compound or a pharmaceutically acceptable salt thereof:

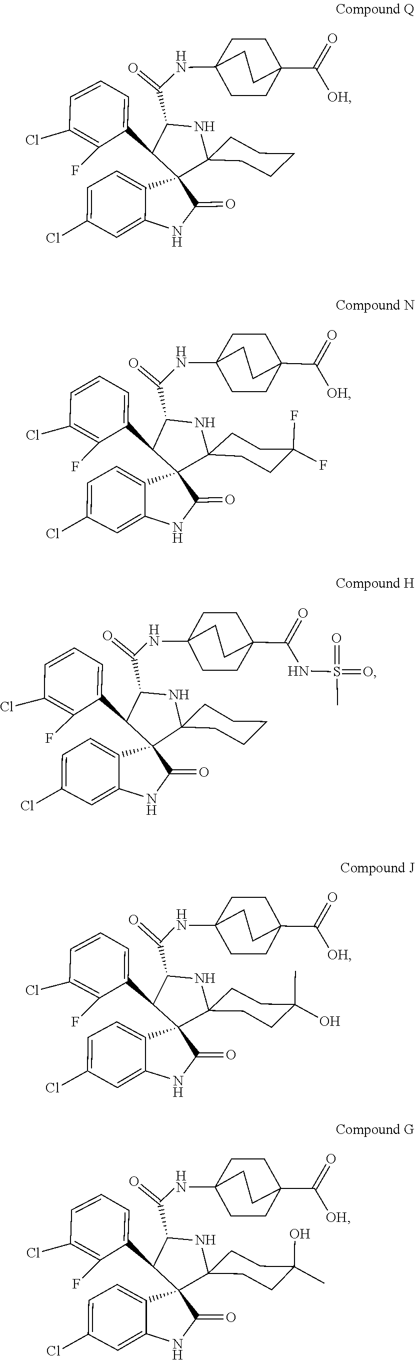 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

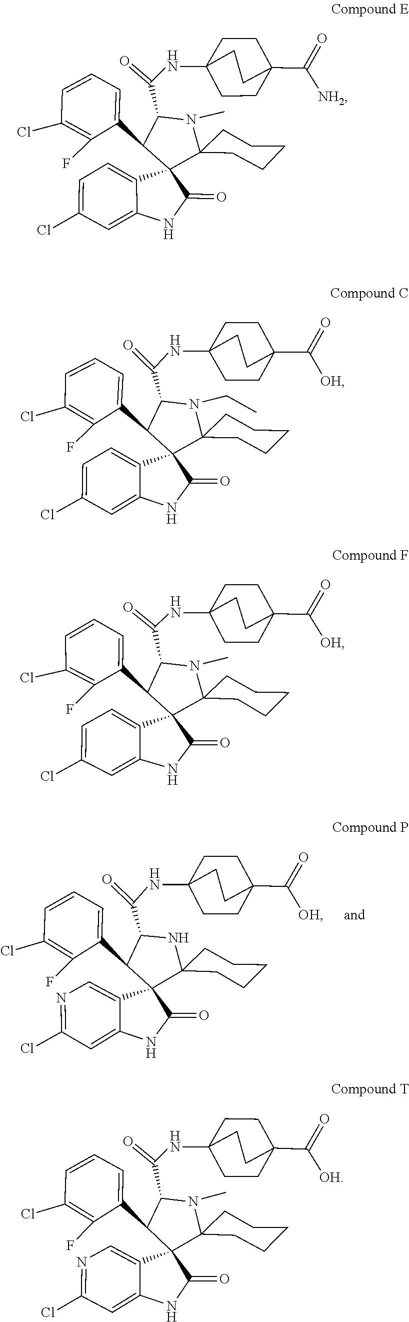 [SMILES: CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2]

30. The method of claim 23, wherein the MDM2 inhibitor is a compound having the following formula or a pharmaceutically acceptable salt thereof

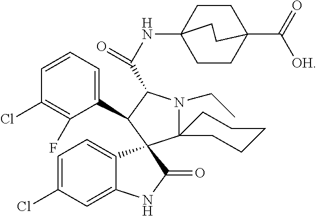 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

31. The method of claim 23, wherein the modulator of an immune checkpoint molecule is a modulator of the immune checkpoint molecule PD-1 or PD-L1, or a PD-1 or PD-L1 binding protein (e.g. anti-PD-1 antibody or anti-PD-L1 antibody).

32. (canceled)

33. The method of claim 23, wherein the modulator of the immune checkpoint molecule is selected from pembrolizumab, nivolumab, atezolizumab, avelumab, durvalumab, AMP-224, AMP-514, BGB-A317, cemiplimab, JS001, CS1001, PDR-001, PF-06801591, IBI-308, pidilizumab, SHR-1210, and TSR-042; preferably, the modulator of the immune checkpoint molecule is pembrolizumab.

34. (canceled)

35. The method of claim 23, wherein the MDM2 inhibitor is a compound having the following formula or a pharmaceutically acceptable salt thereof 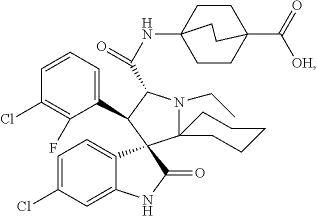 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]
and the modulator of the immune checkpoint molecule is pembrolizumab.

36. The method of claim 23, wherein the MDM2 inhibitor is administered orally every other day;
preferably, the MDM2 inhibitor is administered orally in an effective amount from about 1 mg to about 300 mg every day, preferably in an amount of about 50 mg, 100 mg, 150 mg, 200 mg, 250 mg every other day;
more preferably, the treatment comprises at least one 21-day treatment cycle, wherein the MDM2 inhibitor is administered orally every other day in a patient in need thereof for the first two consecutive weeks (e.g., on day 1, 3, 5, 7, 9, 11 and 13) of a 21-day treatment cycle and is not administered during the third week of the treatment cycle.

37-38. (canceled)

39. The method of claim 35, wherein pembrolizumab is administered via intravenous infusion in an amount of 200 mg on day 1 of the 21-day treatment cycle.

40. The method of claim 23, wherein the cancer is locally advanced, unresectable or metastatic, wherein the immunotherapy is anti-PD-1 or anti-PD-L1 therapy (such as treatment with anti-PD-1 or anti-PD-L1 antibody);
preferably, wherein the subject has unresectable or metastatic melanomas and is refractory or relapse after the treatment with anti-PD-1 or anti-PD-L1 antibody;
or, wherein the subject has unresectable or metastatic NSCLC and is refractory or relapse after the treatment with anti-PD-1 or anti-PD-L1 antibody;
or, wherein the subject has unresectable or metastatic lung adenocarcinoma with STK-11 mutation, and optionally is refractory or relapse after the treatment with anti-PD-1 or anti-PD-L1 antibody;
or, wherein the subject has unresectable or metastatic solid tumors with functional p53 (such as wild-type 53) and ATM mutation (such as germline ATM mutation or somatic ATM mutation);
or, wherein the subject has locally advanced or metastatic liposarcomas with functional p53 (such as wild-type p53) and MDM2 amplification;
or, wherein the subject has unresectable or metastatic urothelial carcinoma without FGFR translocation and/or point mutation and is refractory or relapse after the treatment with anti-PD-1 or anti-PD-L1 antibody;
or, wherein the subject has unresectable or metastatic MPNST.

41-49. (canceled)
